(19) United States
(12) Patent Application Publication
(10) Pub. No.: US 2010/0016547 A1
(43) Pub. Date: Jan. 21, 2010

(54) Glycosilated Peptide and Medicine Comprising It as an Effective Ingredient

(76) Inventors: Takaomi Ito, Hokkaido (JP); Akio Takimoto, Hyogo-ken (JP); Hirofumi Nagatome, Osaka-fu (JP); Masataka Fumoto, Osaka-fu (JP); Taichi Ueda, Osaka-fu (JP); Shin-Ichiro Nishimura, Hokkaido (JP)

Correspondence Address: WENDEROTH, LIND & PONACK, L.L.P., 1030 15th Street, N.W.,, Suite 400 East, Washington, DC, 20005-1503, US

(21) Appl. No.: 12/085,770

(22) PCT Filed: Nov. 29, 2006
(86) PCT No.: PCT/JP2006/323834

§ 371 Date: May 26, 2009

(30) Foreign Application Priority Data
Nov. 30, 2005 (JP) 2005-346905

(51) Int. Cl. C07K 14/605

(52) U.S. Cl. 530/324

(57) ABSTRACT

The present invention related to providing a novel medicine for treating diabetes. It is possible to provide a GLP-1 derivative which is resistant to enzyme degradation by glycosylation of the peptide side chain.

### TECHNICAL FIELD

This invention relates to a novel glycosylated peptide and a medicine comprising it as an effective ingredient. In detail, it relates to a novel glycosylated peptide relating to a glucagon-like peptide-1 (GLP-1), which stimulates insulin secretion and is useful as a medicine for treating diabetes.

### BACKGROUND ART

Glucagon-like peptide-1 (GLP-1) is a peptide hormone secreted from L-cells in the small intestine into blood composed of 30 amino acid residues (Non-patent literature 1). GLP-1 is expected as a candidate of medicine treating diabetes since it stimulates insulin secretion in glucose concentration-dependent manner and has an activity to suppress glucagon secretion, appetite and excretion of gastric emptying (Non-patent literature 2). However, native GLP-1 is degraded in vivo by dipeptidyl peptidase IV (DPP-IV), which releases the N-terminal dipeptide, His-Ala, and inactivates GLP-1 (Non-patent literature 3 and 4), and the half-life of GLP-1 in blood is only several minutes (Non-patent literature 5). Therefore its clinical application was so difficult.

In the past, many GLP-1 derivatives have been reported, which acquired resistance to DPP-IV is acquired by substitution and/or modification of amino acid residues around the cleaved site by DPP-IV. For example, the GLP-1 derivatives which include modification of His⁷ of the N-terminus (Non-patent literature 6-8), amino acid substitution of Ala⁸ (Non-patent literature 9-11) or Glu⁹ (Non-patent literature 12) have been reported.
Non-patent literature 1: Lancet. 1987 2 13004
Non-patent literature 2: Regul Pept 2005; 128: 135-48
Non-patent literature 3: Eur J Biochem 1993; 214: 829-35
Non-patent literature 4: J Biol Chem 1997; 272: 21201-6
Non-patent literature 5: Diabetologia 1998; 41: 271-278
Non-patent literature 6: Regul Pept 2001; 96: 95-104
Non-patent literature 7: Regul Pept 2000; 86: 103-111
Non-patent literature 8: Regul Pept 1999; 79: 93-102
Non-patent literature 9: JBC 2004; 279: 3998-4006
Non-patent literature 10: J Endocrinol 1998; 159: 93-102
Non-patent literature 11: Metabolism 1999; 48: 252-258
Non-patent literature 12: Biol Chem 2003; 384: 1543-1551

### DISCLOSURE OF INVENTION

### Problem to be Solved

The objective of the present invention is to provide a derivative of GLP-1 related peptide, which has a long half-life in blood and is useful as a stimulator of insulin secretion.

### Means to Solve the Problem

The inventors have found that it is successful to provide a DPP-IV tolerant GLP-1 derivative while maintaining an activity to stimulate insulin secretion by glycosylation of GLP-1 related peptides, and completed the present invention.

### EFFECT OF INVENTION

The glycosylated GLP-1 related peptides have a long half-lives in blood and continuously stimulate insulin secretion.

### BRIEF DESCRIPTION OF DRAWINGS

In the FIGURE, the result of a MS spectroscopy was shown as to the prepared glycosylated peptides.

FIG. 1: The result of MS spectroscopy as to glycosylated GL34N was shown.

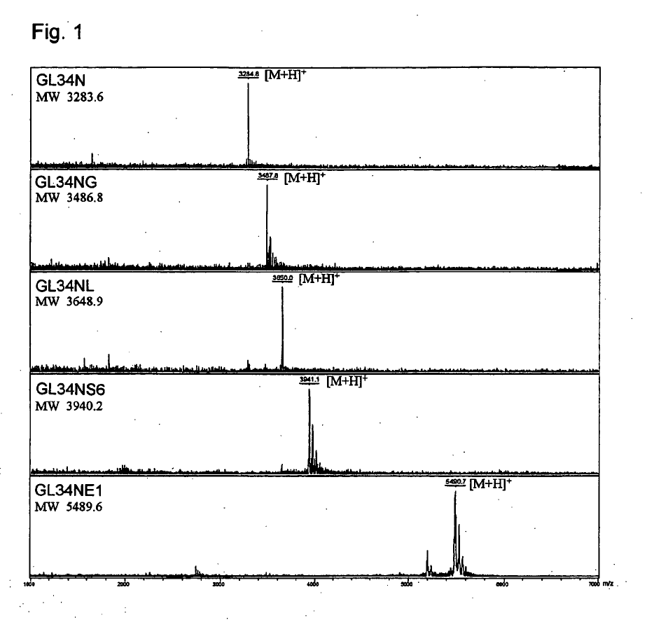

### BEST MODE FOR CARRYING OUT THE INVENTION

A glycosylated peptide provided by the present invention is a glycopeptide in which glycochains set forth below are attached to GLP-1 related peptides. The term of “GLP-1 related peptide” means GLP-1 (7-36) amide of the formula (I) or Excendin-4 of the formula (II);

| | |
|---|---|
| | 7 36 |
| (I): | HAEGTFTSDVSSYLEGQAAKEFIAWLVKGR-NH2 |
| (II): | HGEGTFTSDLSKQMEEEAVRLFIEWLKNGGPSSGAPPPS-NH2 |
| | 1 39 |

and a peptide in which one to eleven, preferably one to six, and more preferably one to three amino acid(s) are deleted, substituted and/or added in the amino acid sequence is included therein.

GLP-1 related peptide has an activity to stimulate insulin secretion and His⁷, Gly¹⁰, Phe¹², Thy¹³, Asp¹⁵, Phe²⁸ and Ile²⁹ in the peptide (I), and His¹, Gly⁴, Phe⁶, Thr⁷, Asp⁹, Phe²² and Ile²³ in the peptide (II) are important for expressing the activity, and a peptide in which the deletion, substitution and/or addition does not affect these residues is preferable.

Also, His⁷ may be replaced with an amino acid analogue having a heterocyclic ring in the side chain. Specifically it can be replaced with the analogue having the side chain of the next formula:

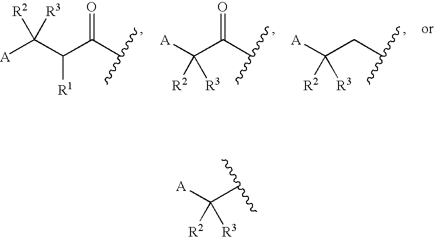

wherein R¹, R² and R³ are independently a hydrogen atom, lower alkyl optionally substituted with aryl, lower alkylcarbonylamino, hydroxyl, lower alkyloxy, a halogen atom, a lower alkylsulfonyl or trifluoromethyl, or R¹ and R² may form a single bond;
wherein aryl may be substituted with a substituent selected from amino, hydroxyl, lower alkyl, lower alkyloxy, a halogen atom, lower alkylsulfonyl, lower alkylcarbonylamino and trifluoromethyl;

A is a cyclic group of

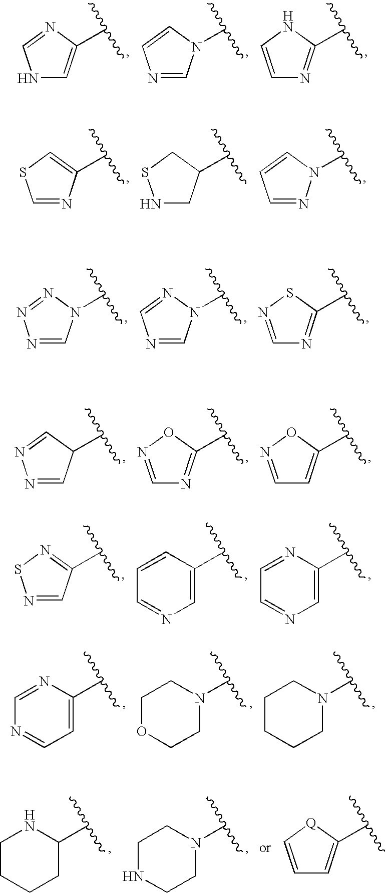

wherein Q is a nitrogen atom, an oxygen atom or a sulfur atom;
and the said cyclic group may be substituted with one or more of substituents selected from amino, nitro, hydroxyl, lower alkyl, lower alkyloxy, a halogen atom, trifluoromethyl and aryl.

Next, embodiment of glycosylation is set forth. Glycochain may be attached directly or through a linker to an functional group of amino acid side chain. Specifically as shown in the next formula,

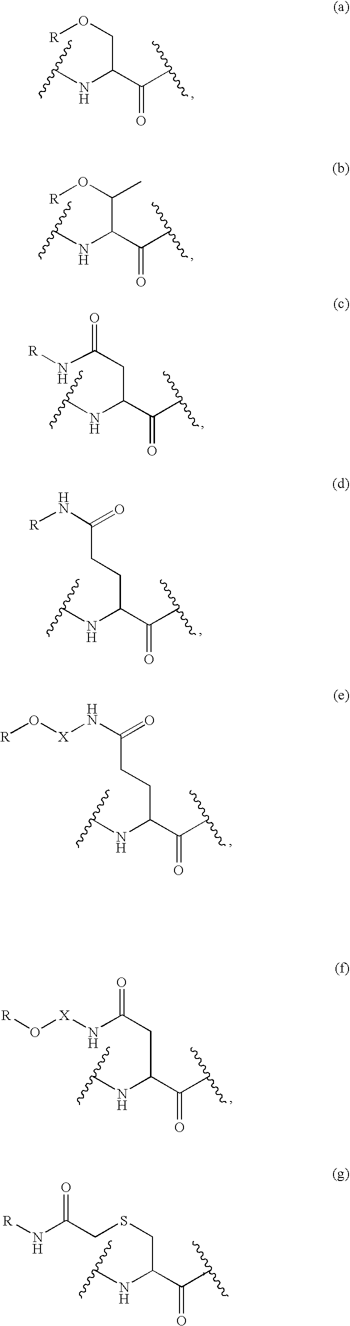

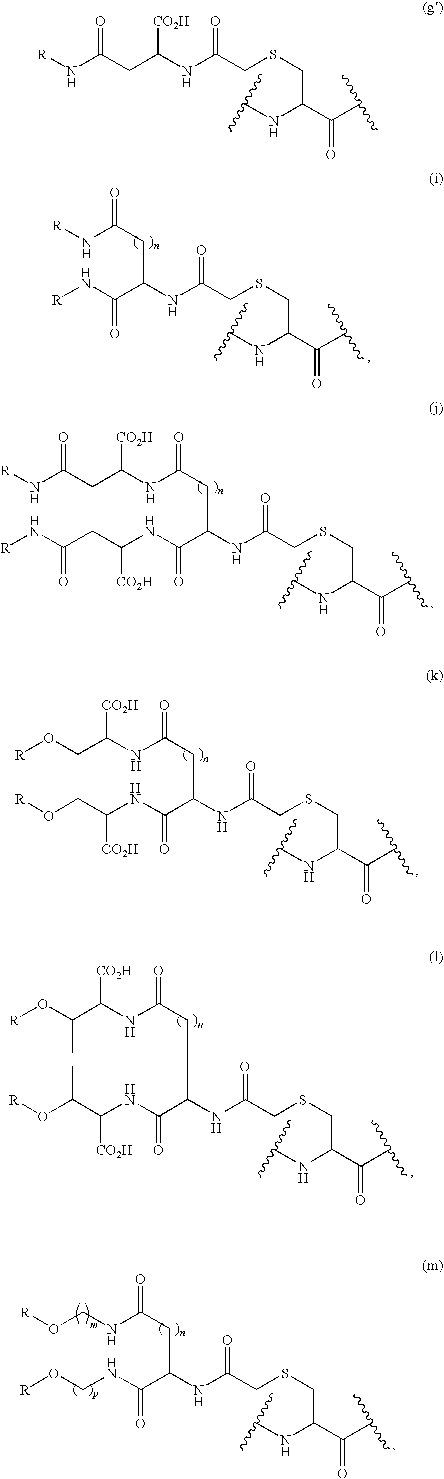

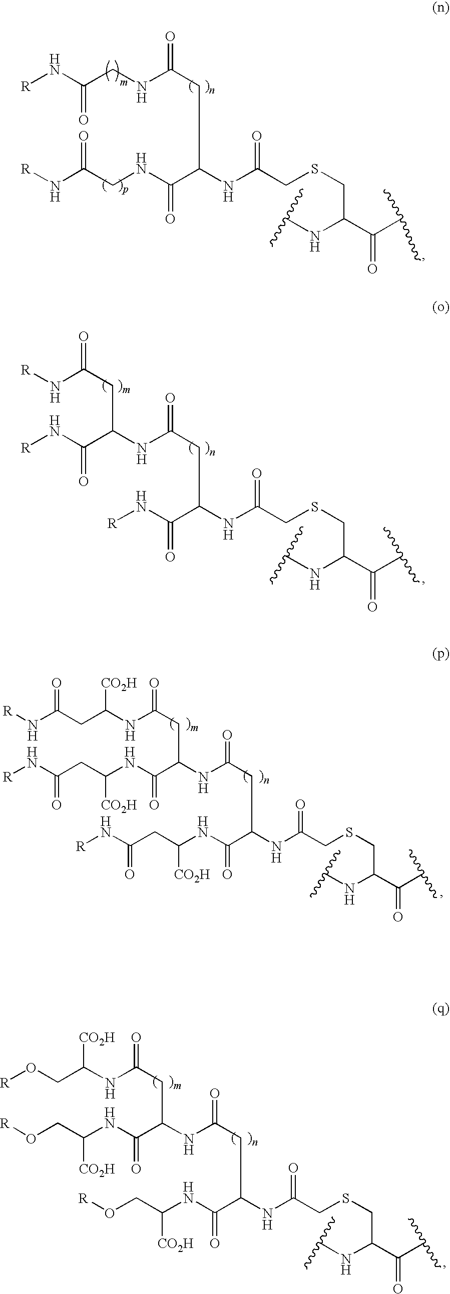

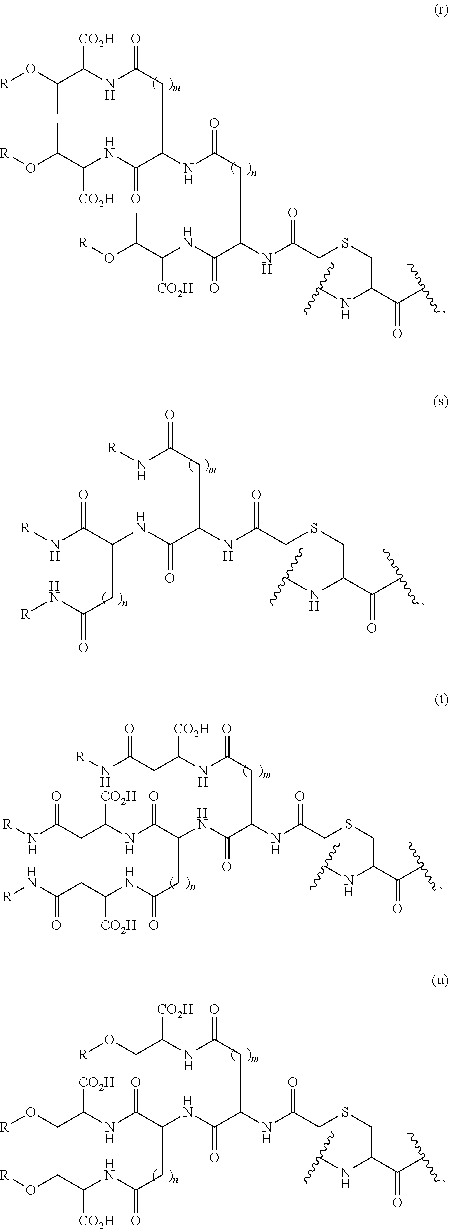

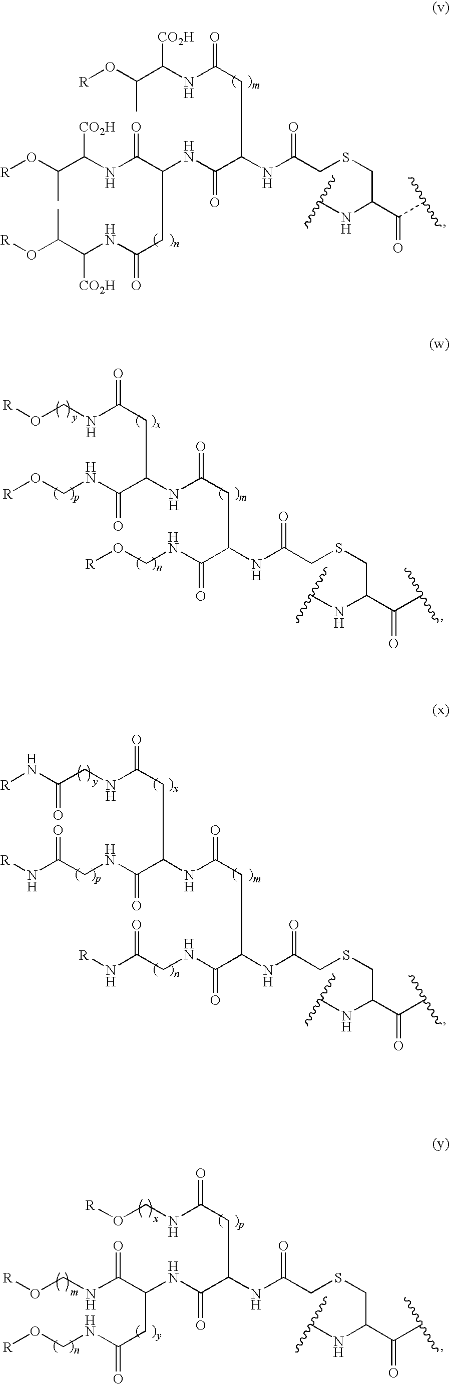

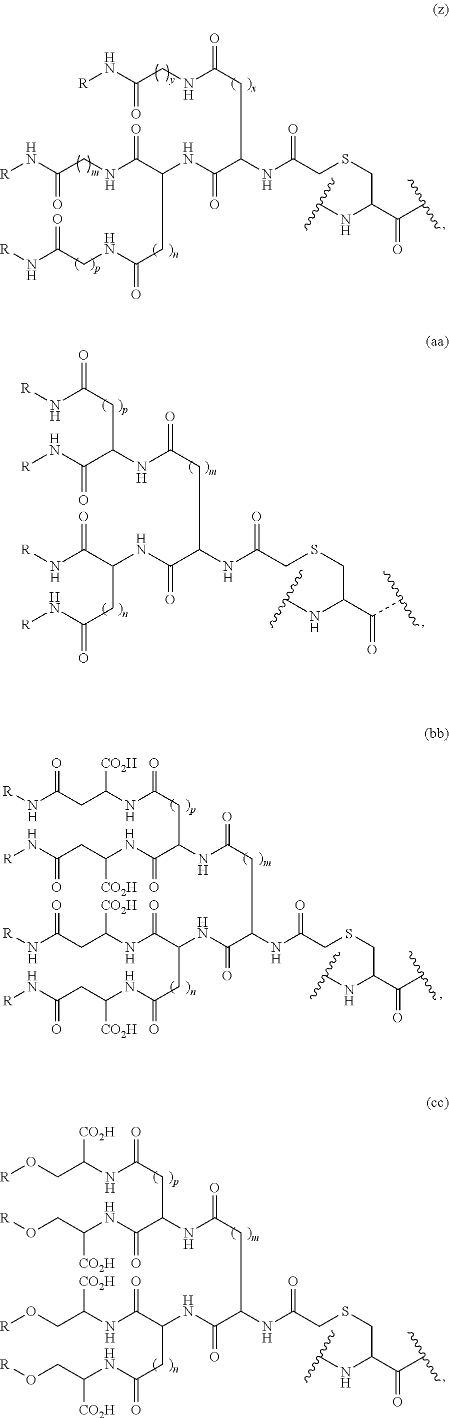

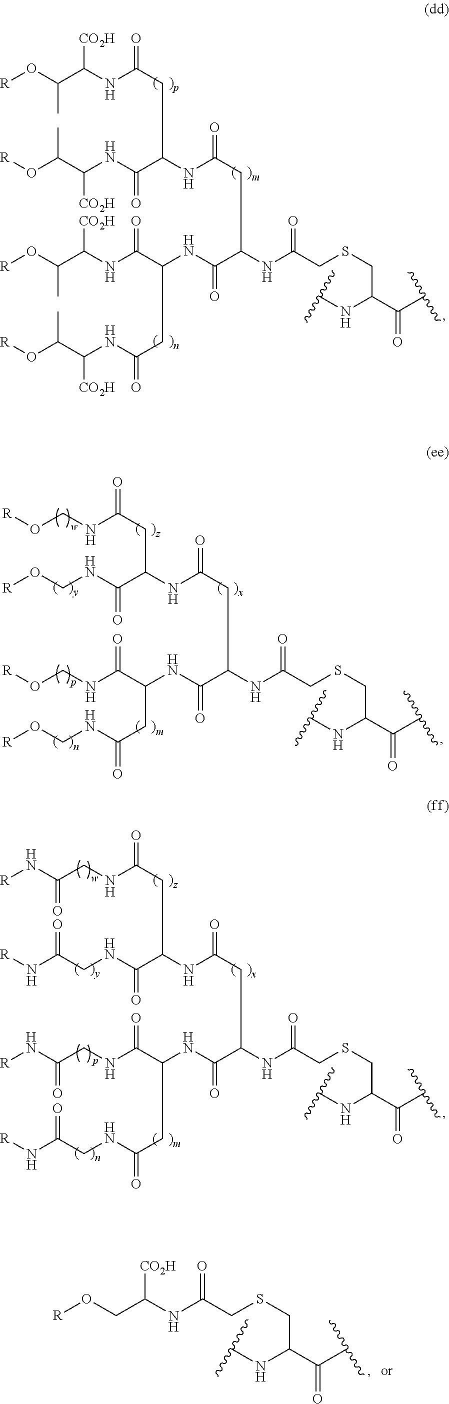

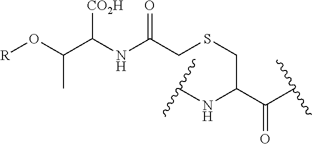

wherein R is independently a glycochain, X, Y and Z are linkers, m, n, p, w, x, y, z are integers of 1 to 10,
glycosylation is possible at the side chain of Asp, Asn, Glu, Gln, Ser, Thr and/or Cys. The term “glycosylated Ser, Thr, Asp, Asn, Glu, Gln and Cys” include groups shown by the formulae above.

As a linker, X include optionally substituted methylene.

It is known that the penultimate amino acid residue at the N-terminus in the natural GLP-1 (7-36)amide is enzymatically cleaved by DDP-IV. Accordingly, it is preferable that the glycosilation site is as close to the cleaved site as possible unless it affect the activity.

In order to prevent the degradation caused by DDP-IV while maintaining the activity of the natural GLP-1 (7-36)amide of (I), it is preferable to introduce one to three glycosylated amino acid(s) described above at position-20 or position later. For example, introduction of the glycosylated amino acid(s) at position-26, -34 and/or -37 is preferable. That is, it is preferable not to delete or substitute His⁷, Gly¹⁰, Phe¹², Th¹³, Asp¹⁵, Phe²⁸ and Ile²⁹ and to introduce one to three glycosylated amino acid(s) described above at position-20 or position later. Especially, it is preferable to introduce the glycosylated amino acid(s) at position-26, -34 and/or -37.

In the formula (III), a peptide in which Xaa is His and one to three residue(s) between Xjj and Xyy is substituted with the glycosylated amino acid(s) is preferable (in this case, a sequence of Xzz to Ygg does not exist and amino acid other than Xaa to Ygg is not deleted or substituted.) Amino acid of the formula (I) is preferable, if it is not variated.

In order to improve the prolonged activity while maintaining the activity of excendin-4 of (II), it is preferable to introduce one to four of the glycosylated amino acid(s) above at position 17 or position later. For example, introduction of the glycosylated amino acid(s) at position-21, -28, -35 and/or 40 is preferable. That is, it is preferable not to delete or substitute His¹, Gly⁴, Phe⁶, Thr⁷, Asp⁹, Phe²² and Ile²³ in the formula (II) and to introduce one to four of the glycosylated amino acid(s) described above at position-17 or position later. Especially, introduction of the glycosylated amino acid(s) at position-21, -28, -35 and/or 40 is preferable.

In the formula (III), a peptide in which Xaa is His, and one to four of the glycosylated amino acid(s) is introduced, especially introduced at Xqq, Xvv, Ycc and/or position-46 is preferable. Amino acid of the formula (II) is preferable, if it is not variated (in this case, amino acid other than Xaa to Ygg is not deleted or substituted.).

A kind of glycochain is not limited in peptide modification. Examples of the glycochain used in the present invention are set forth below;

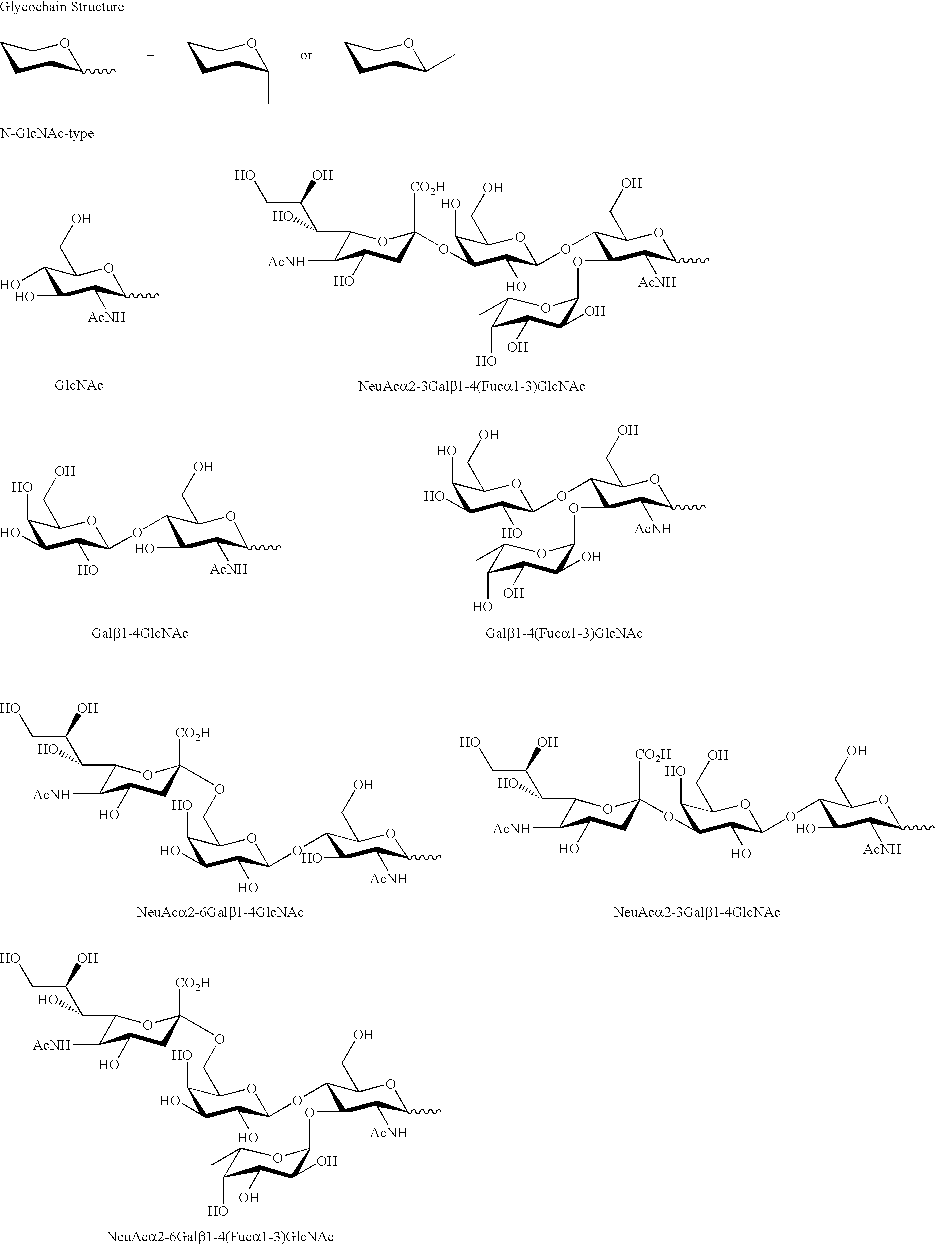

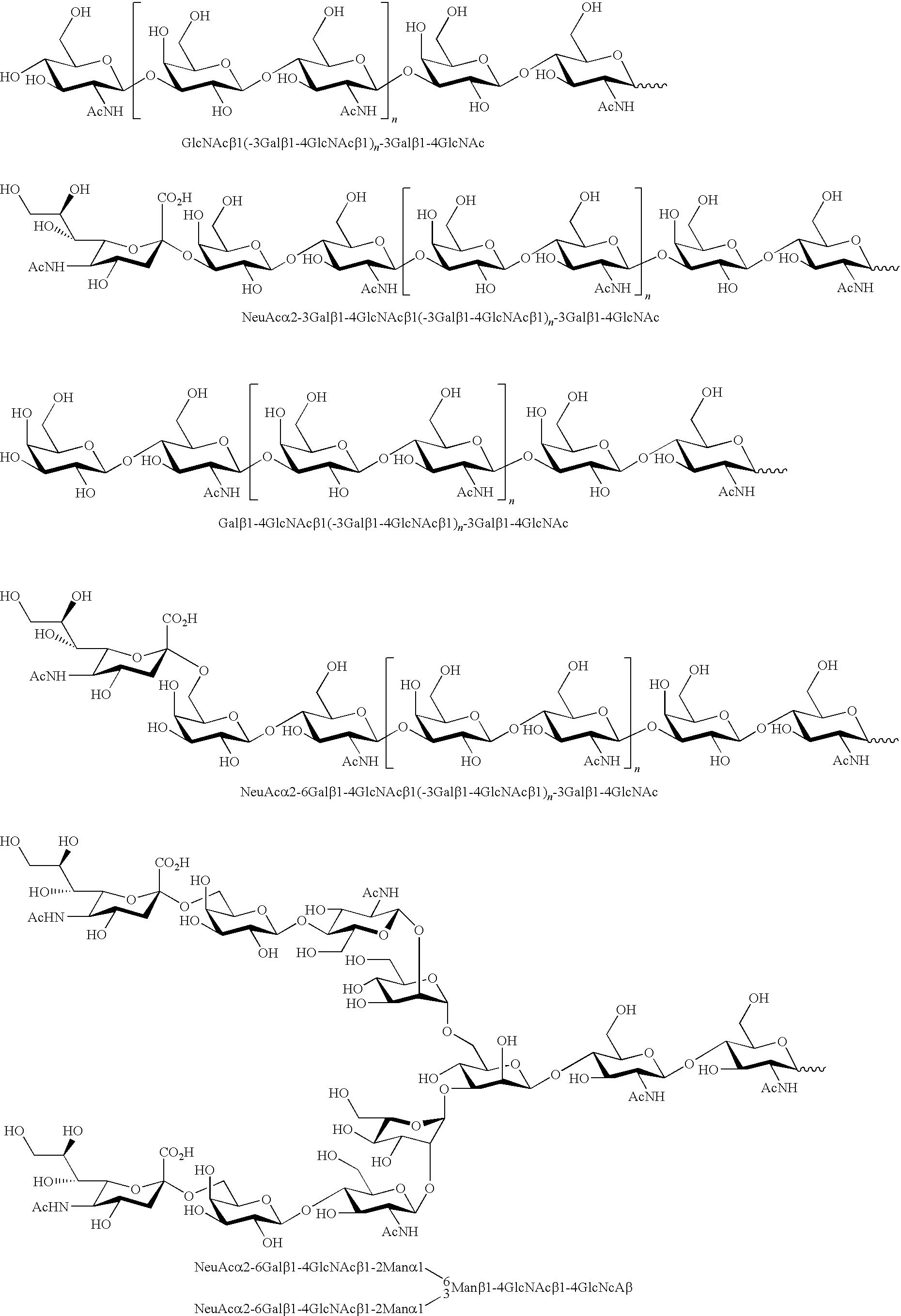

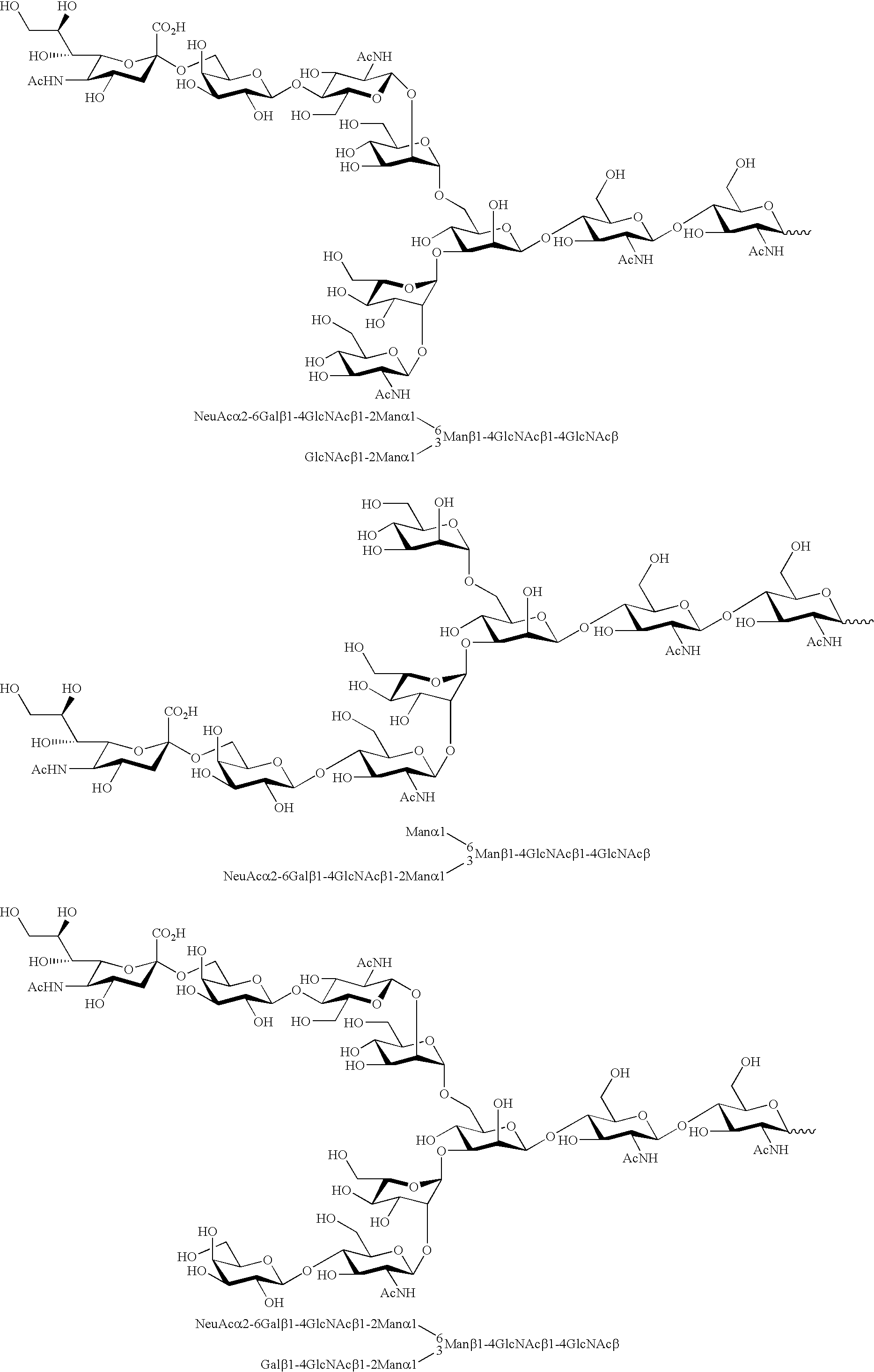

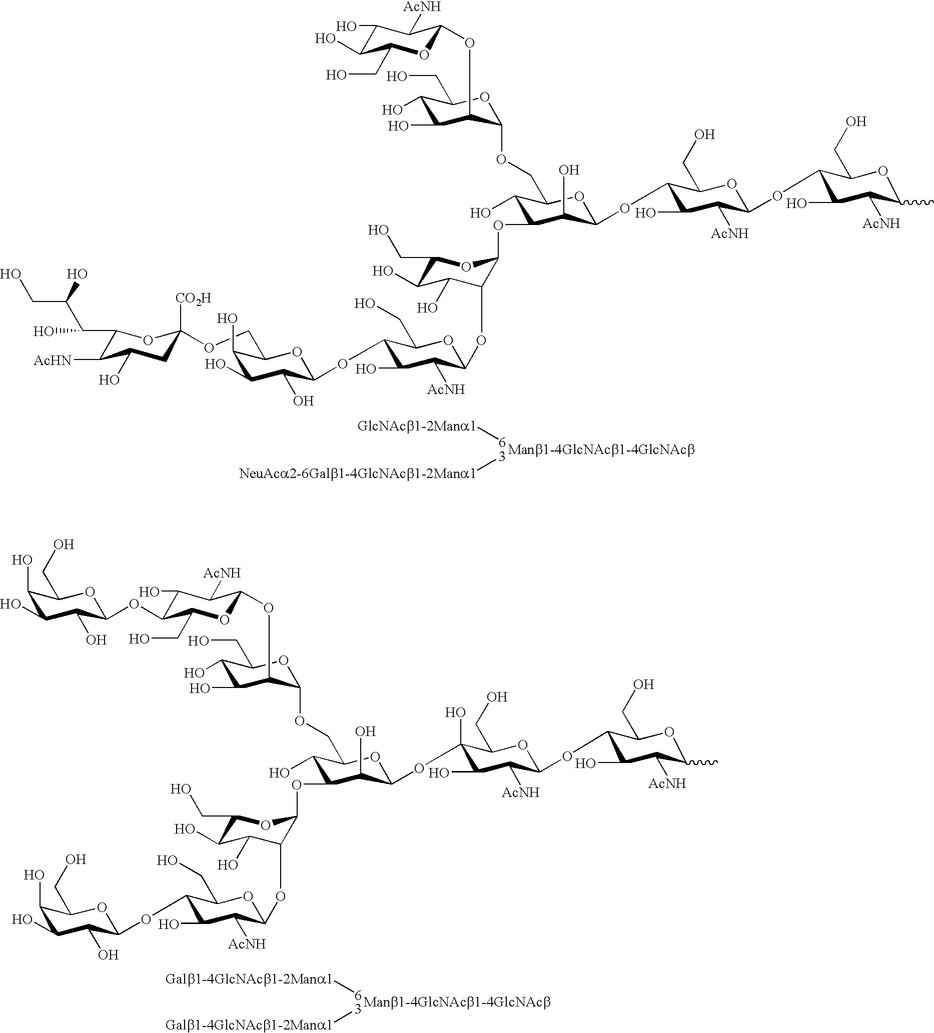

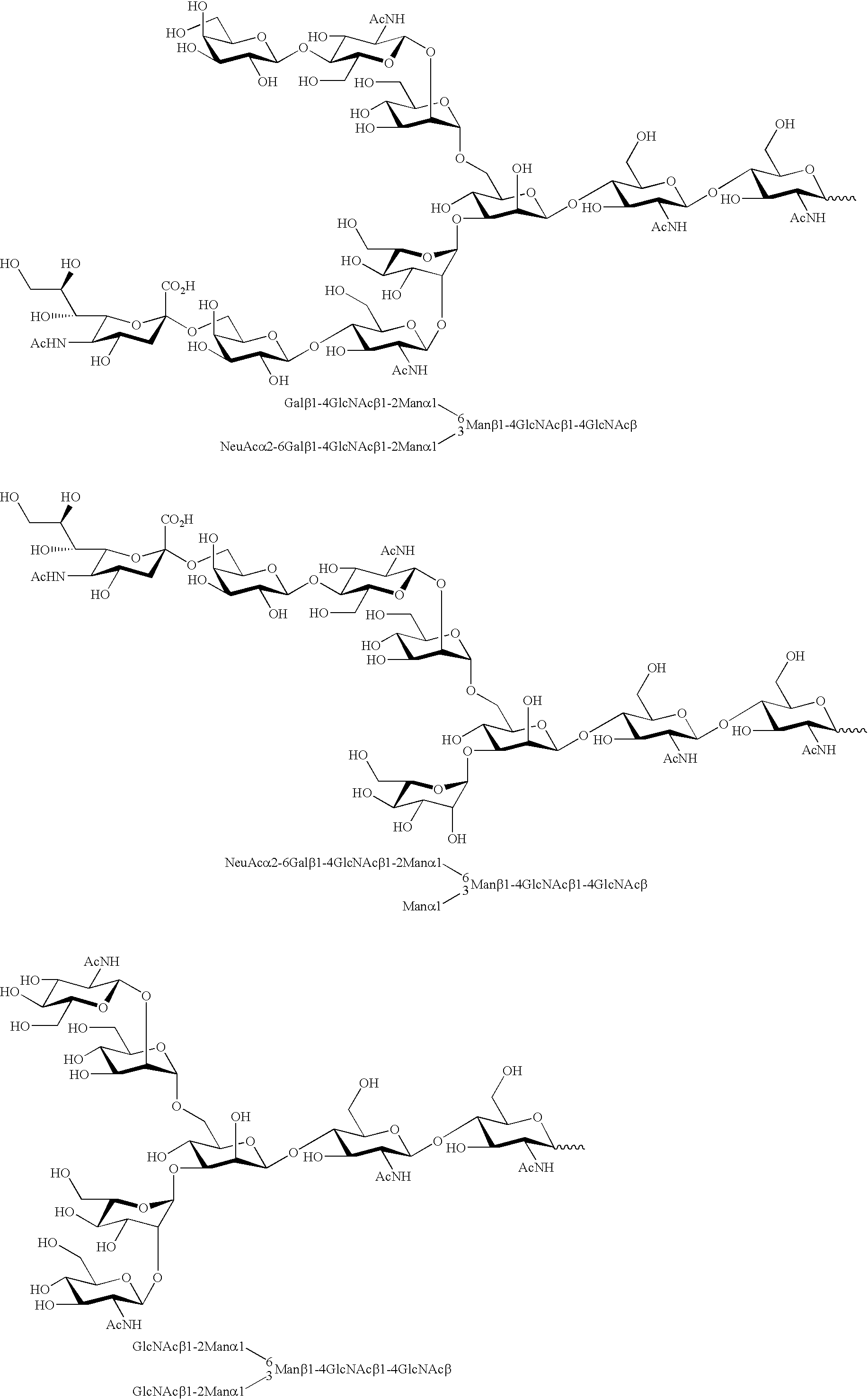

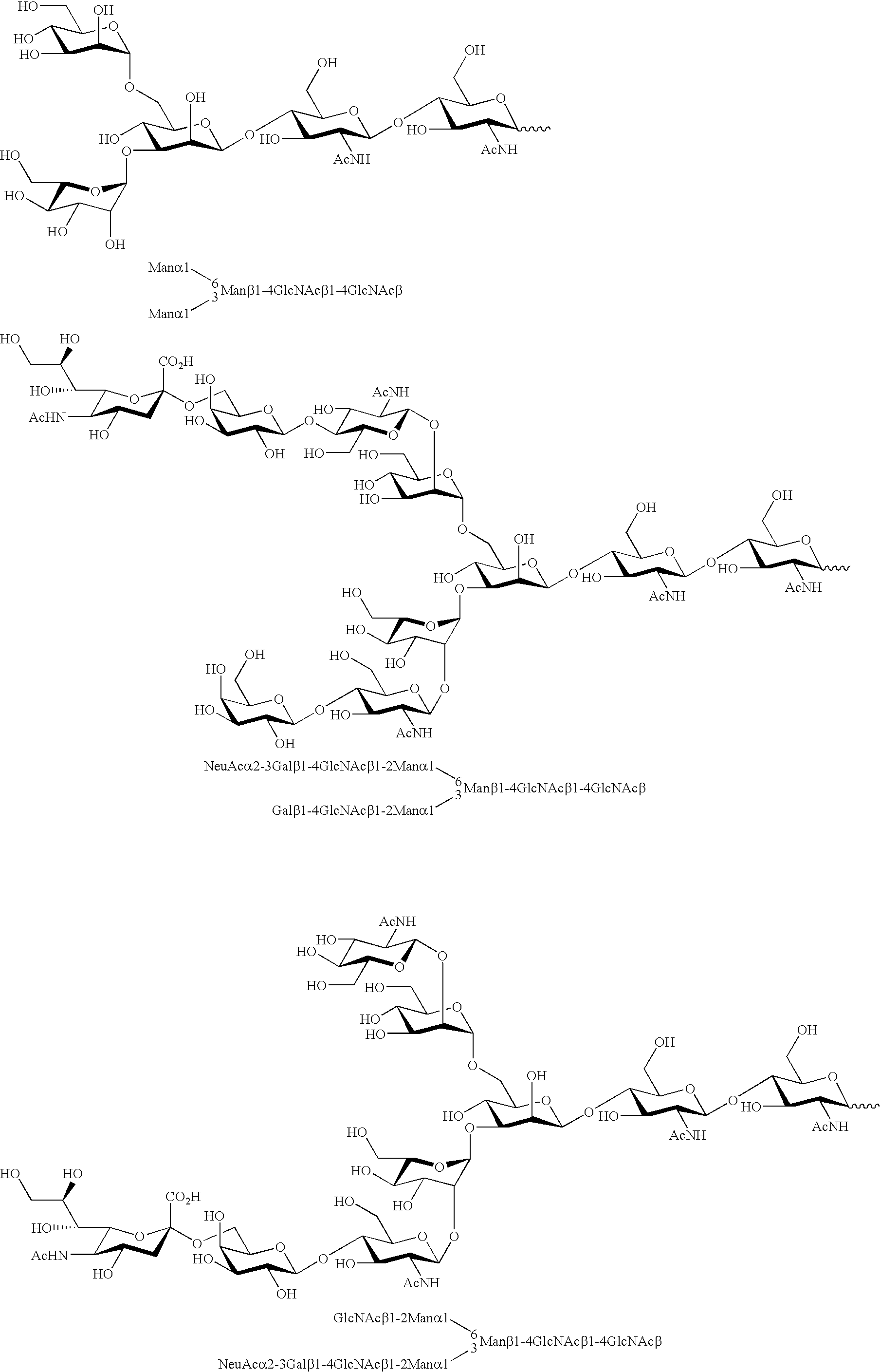

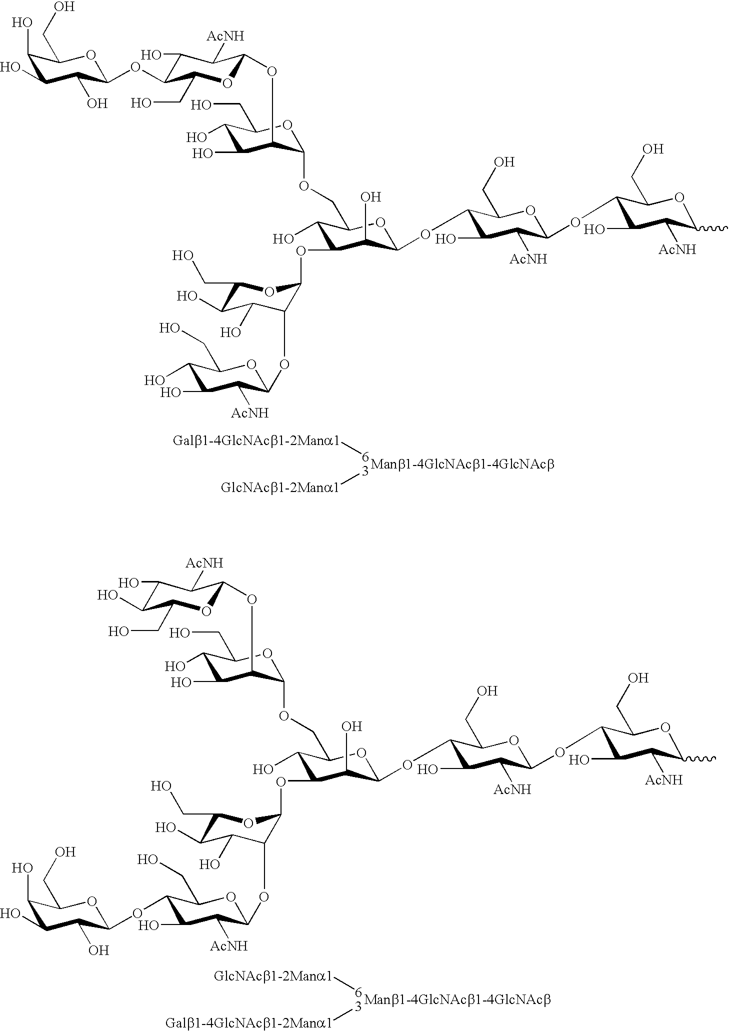

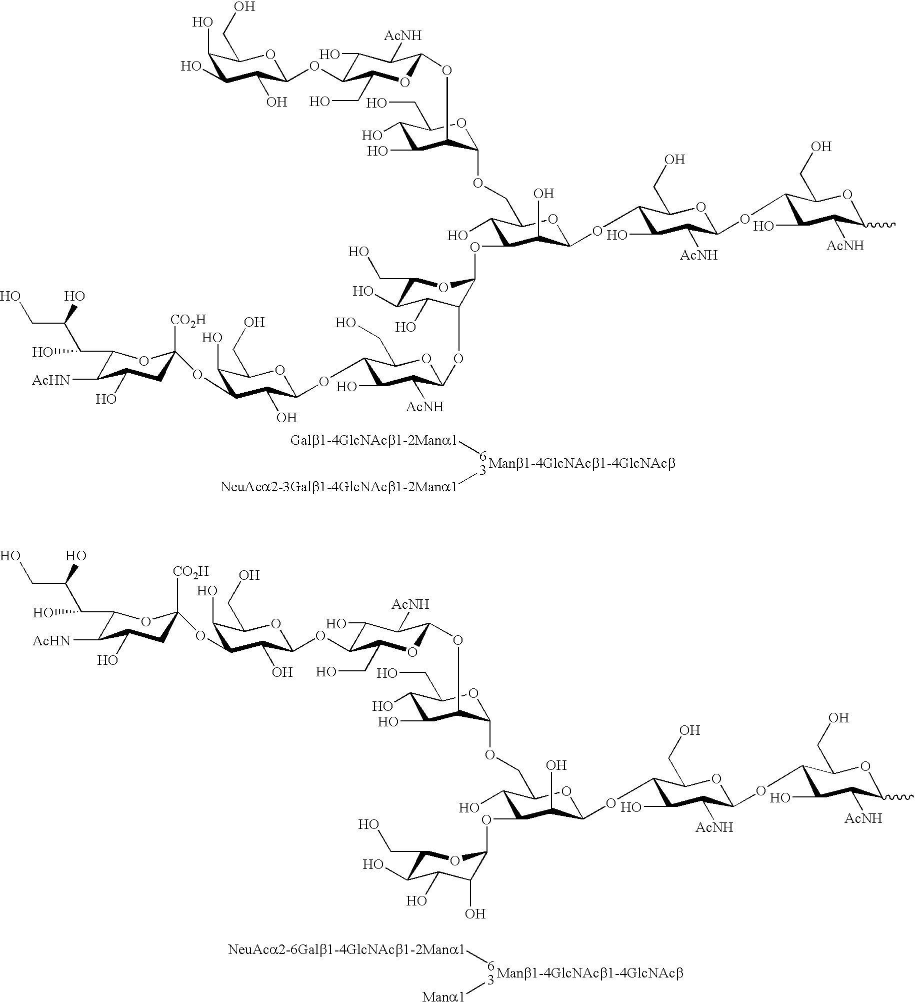

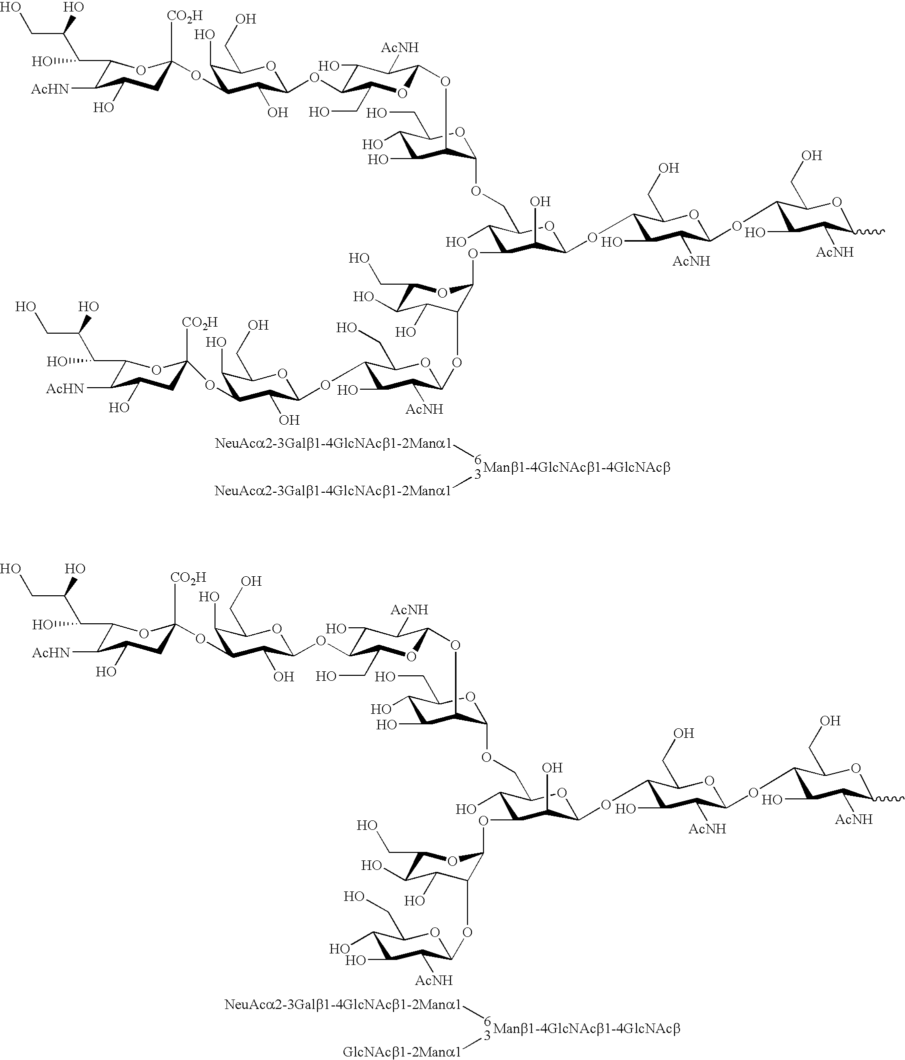

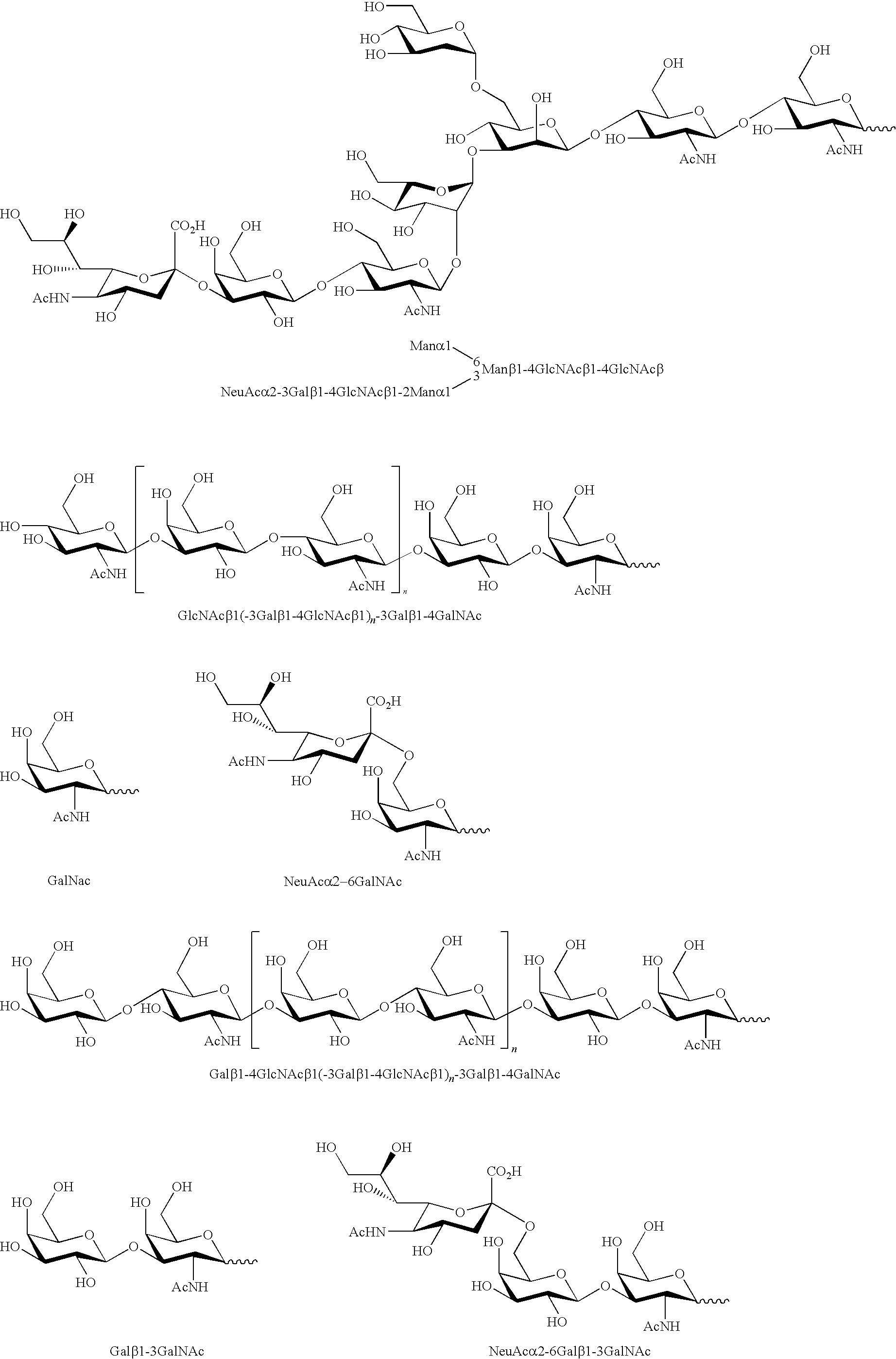

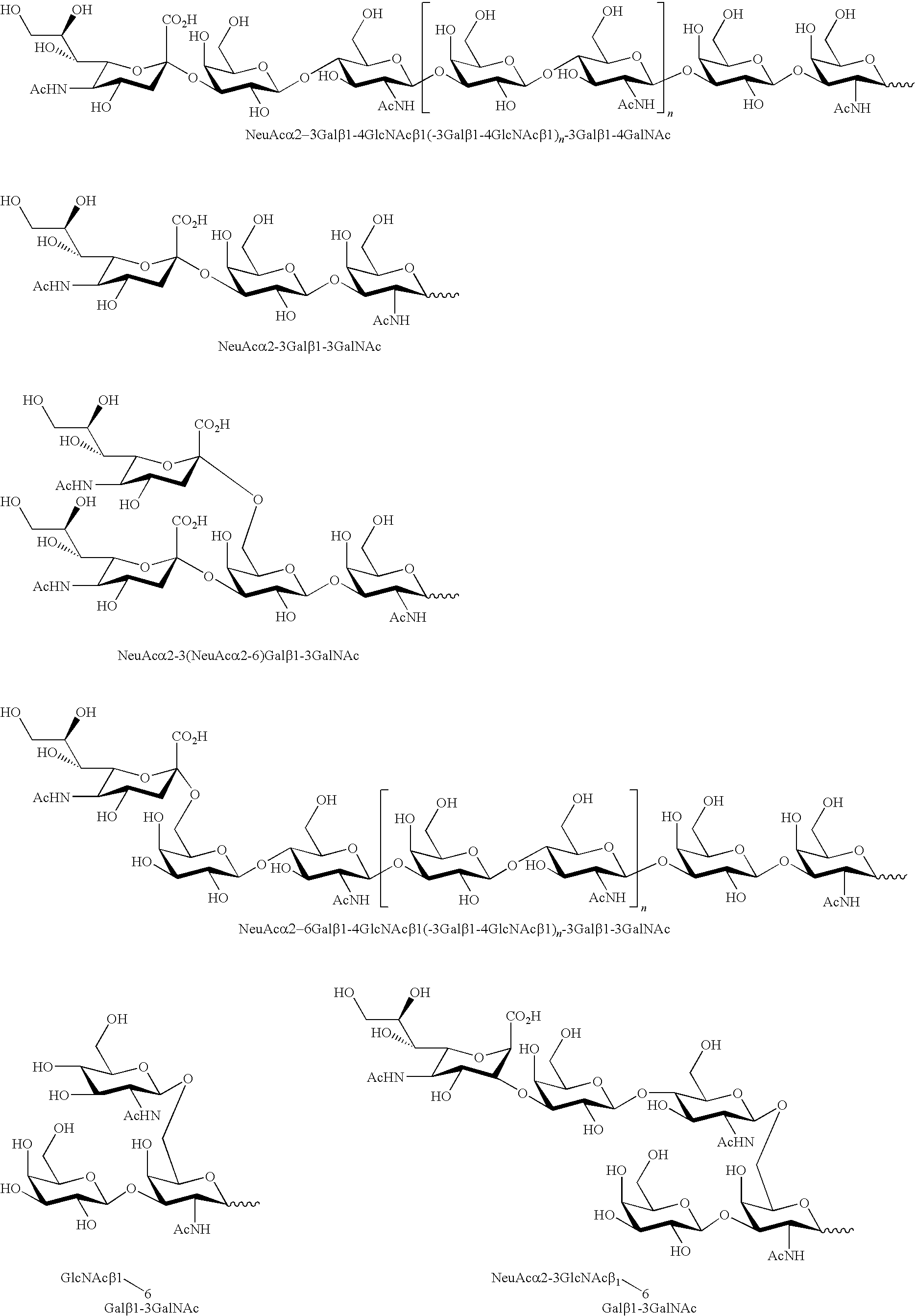

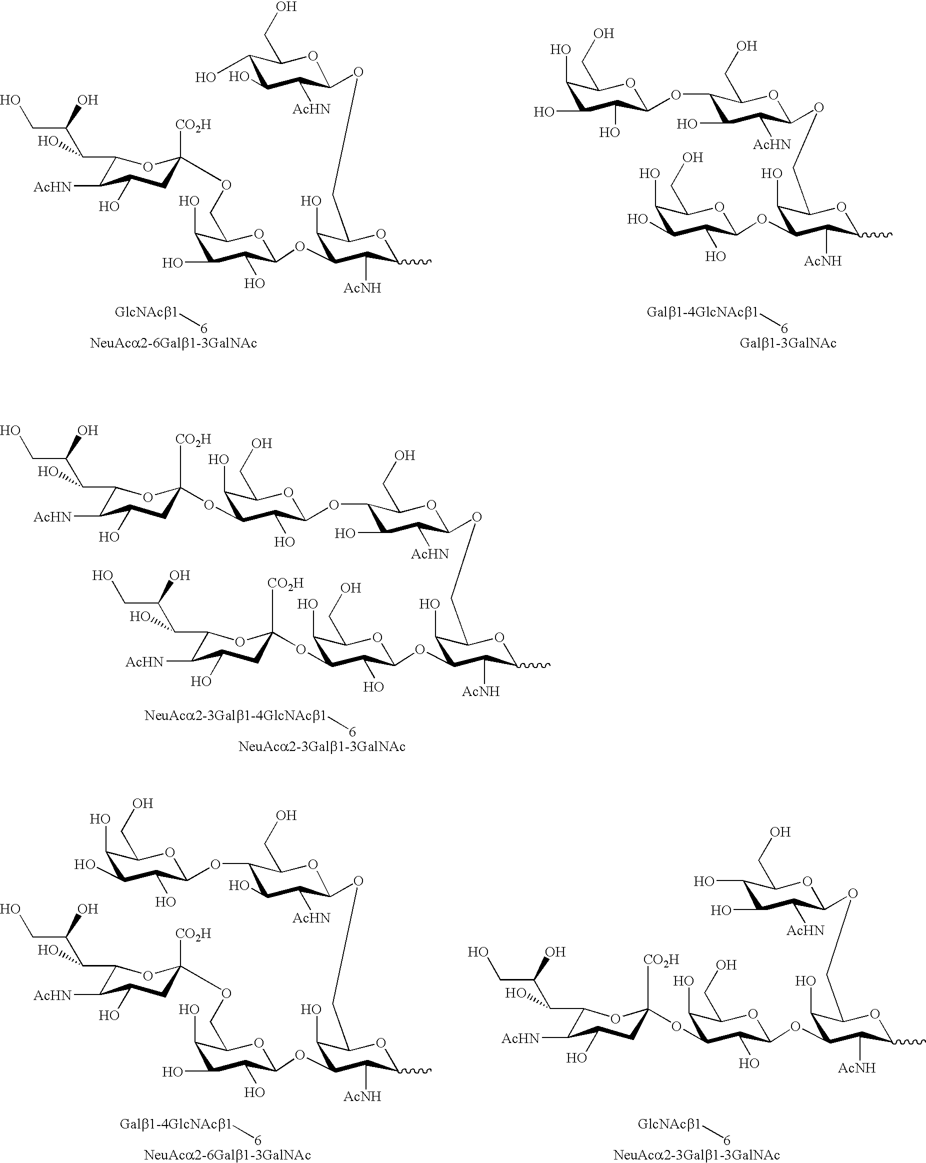

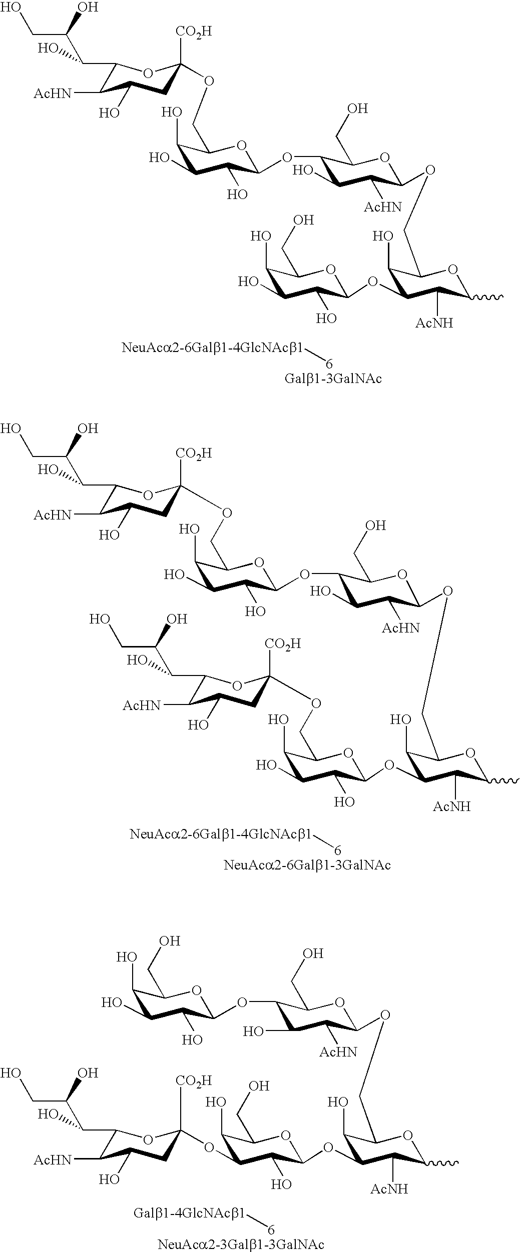

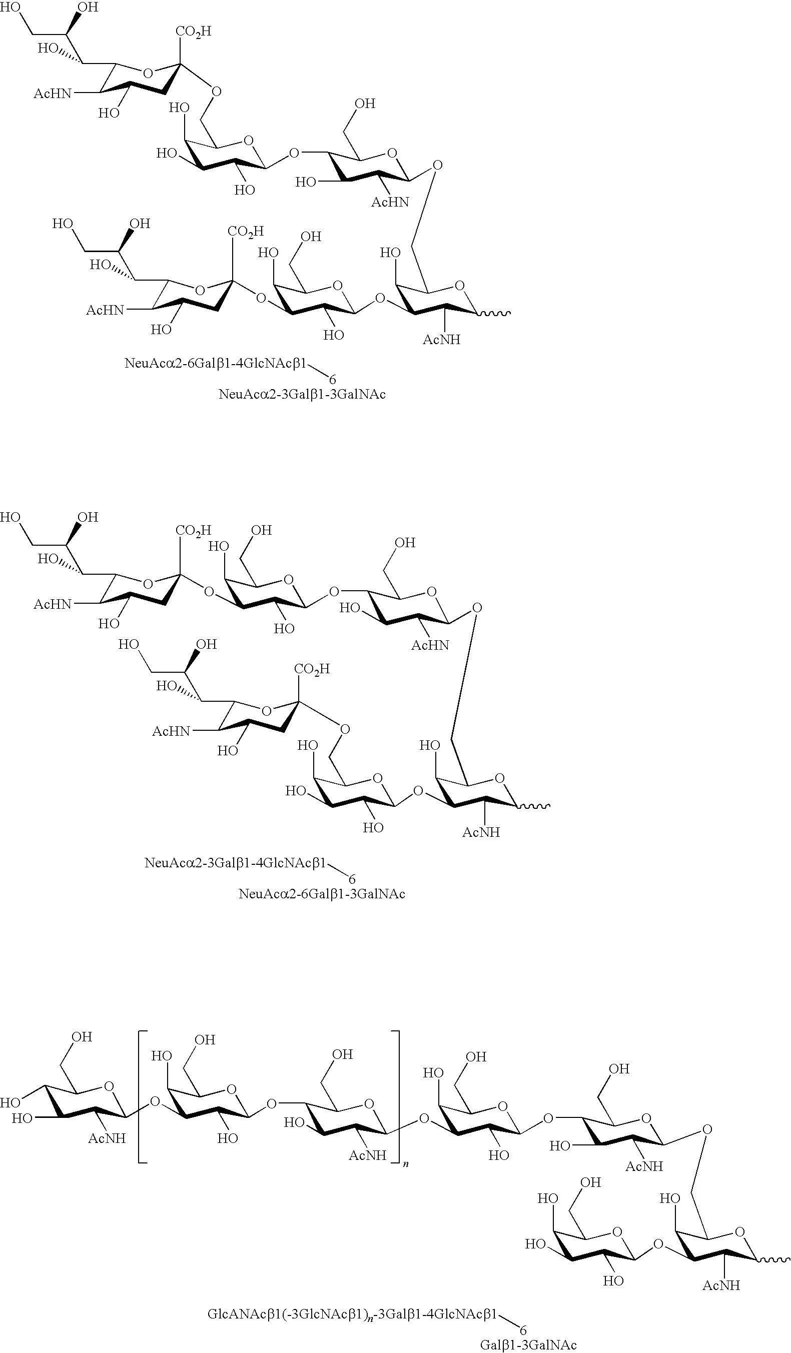

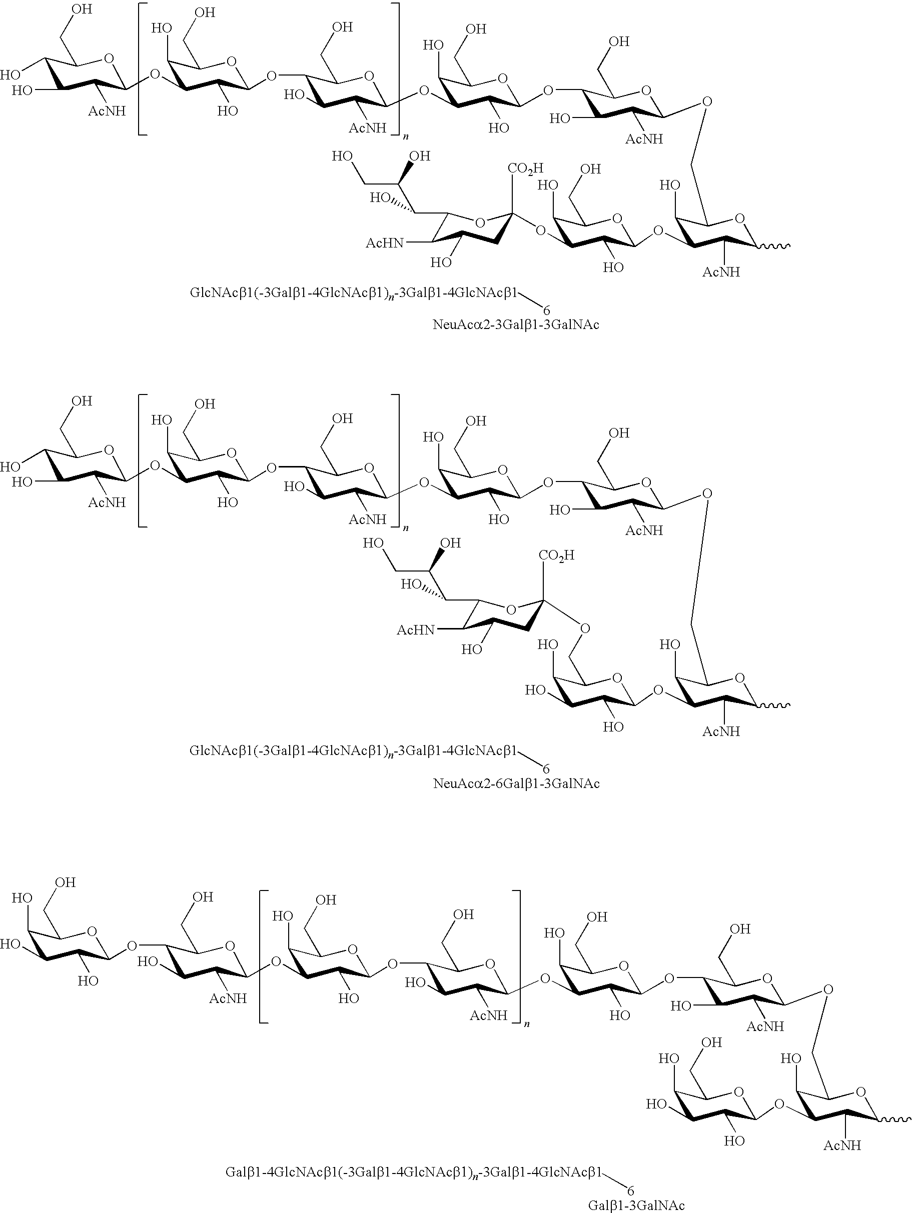

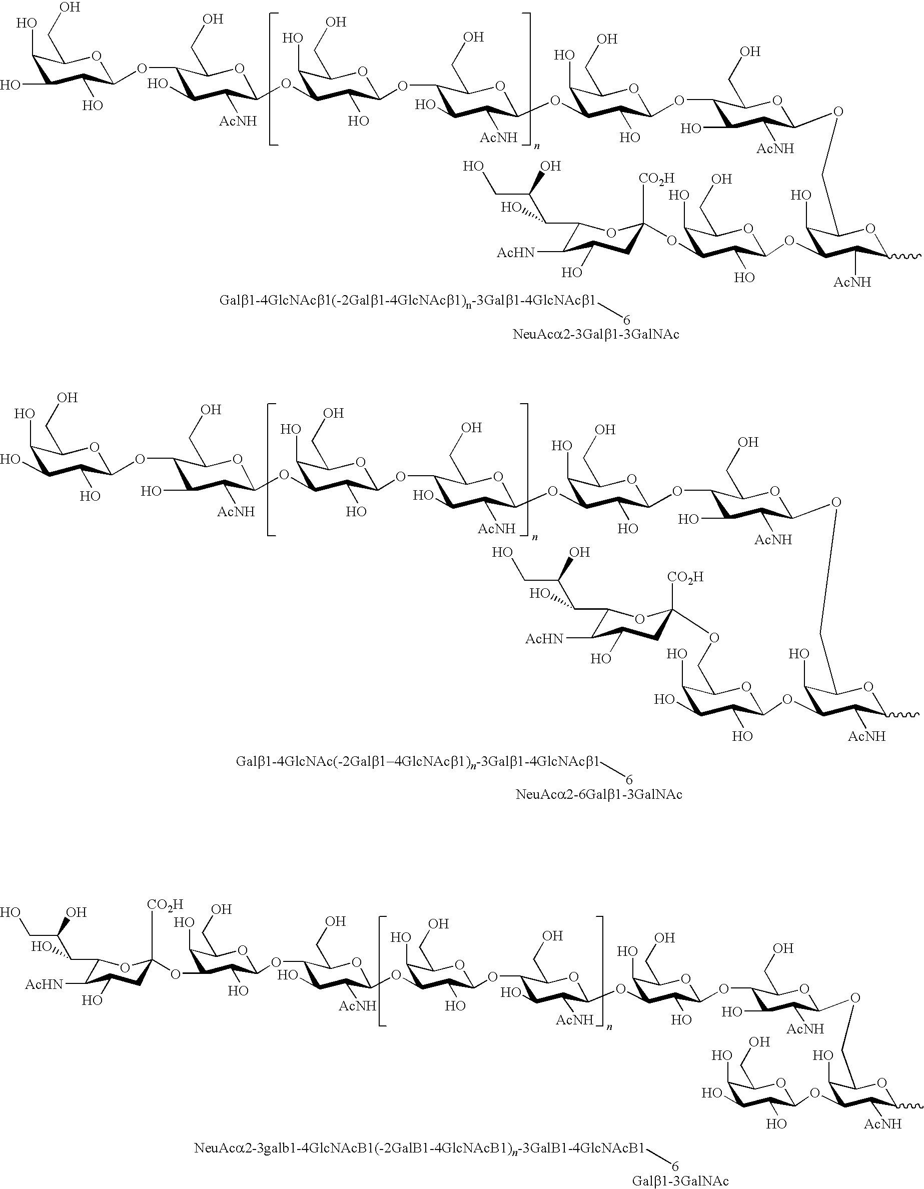

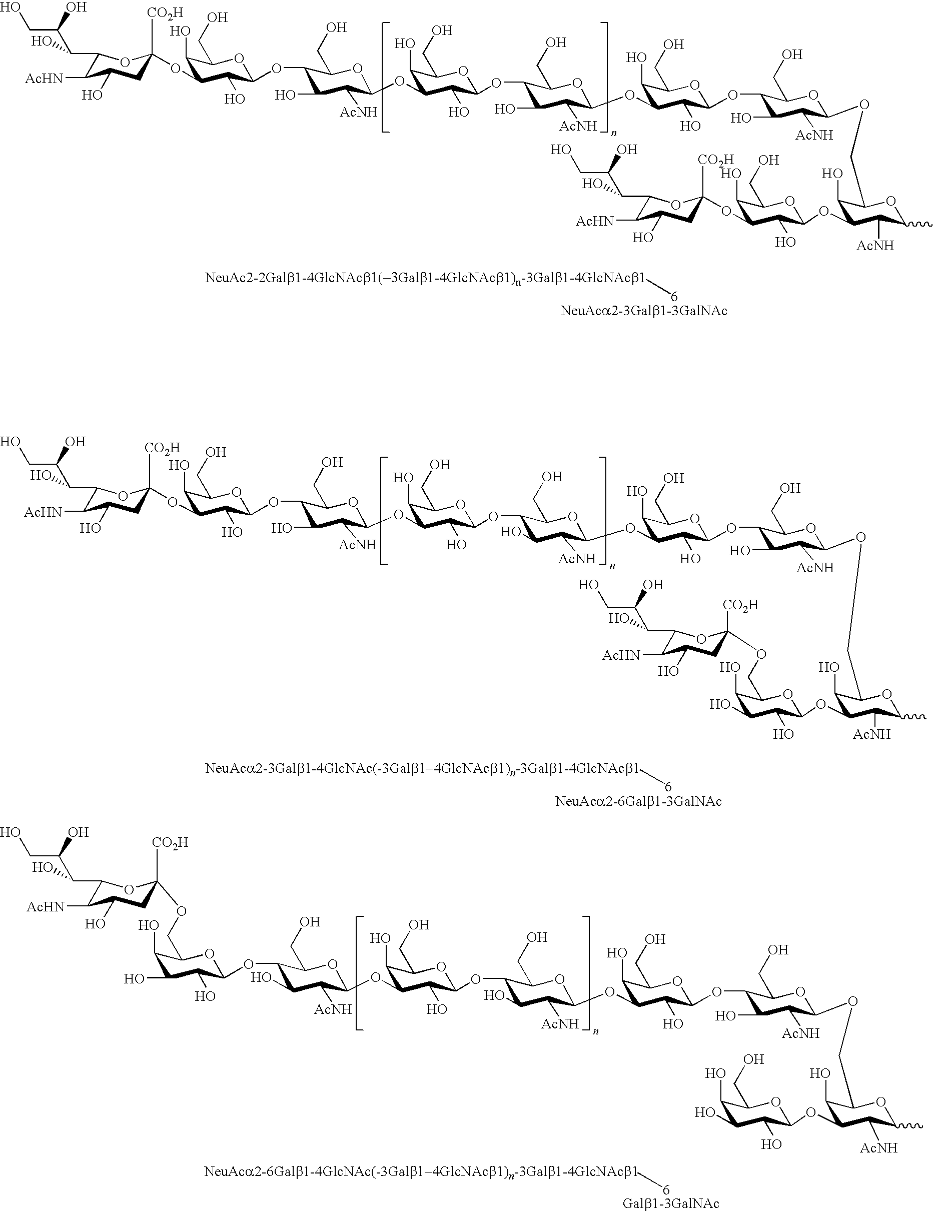

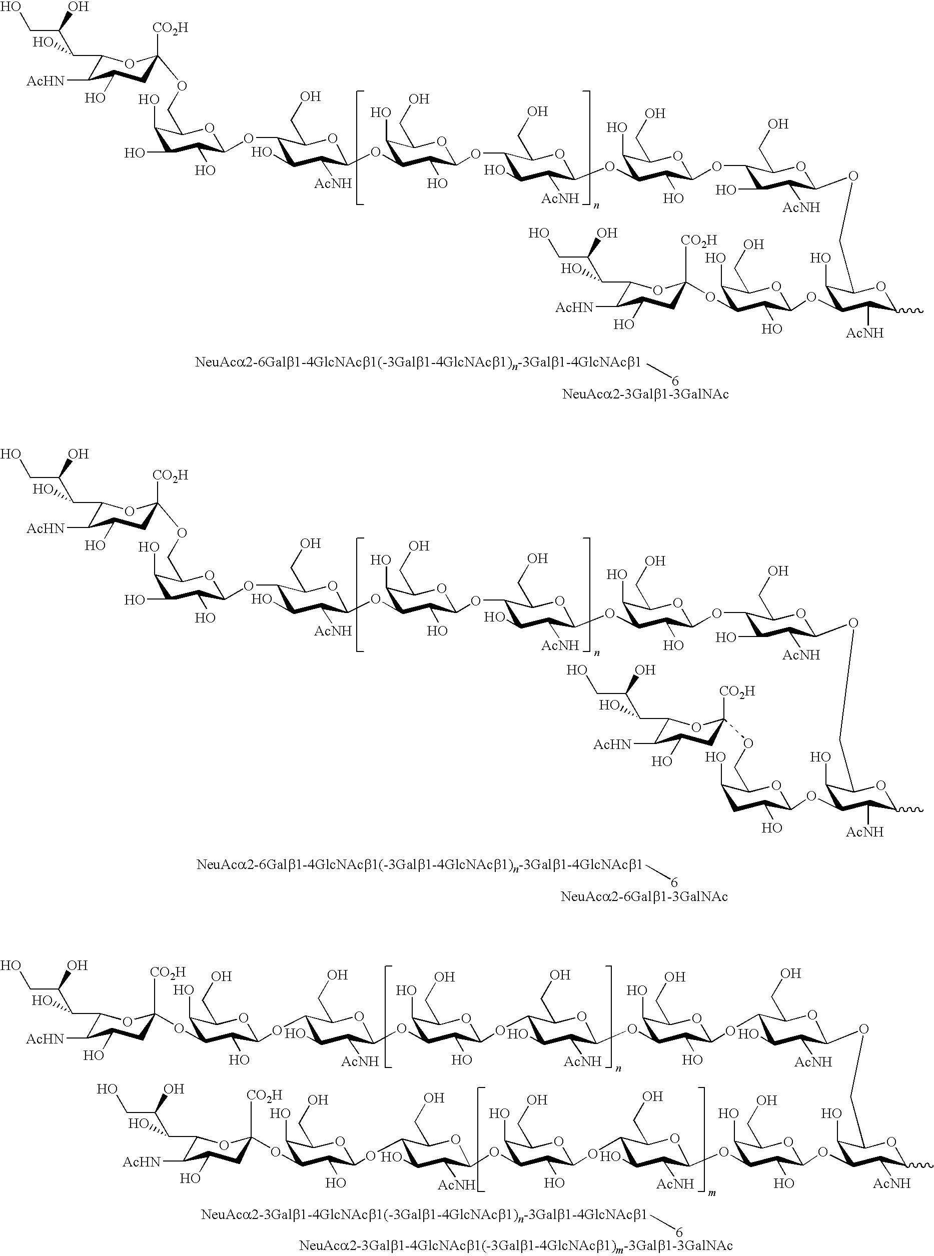

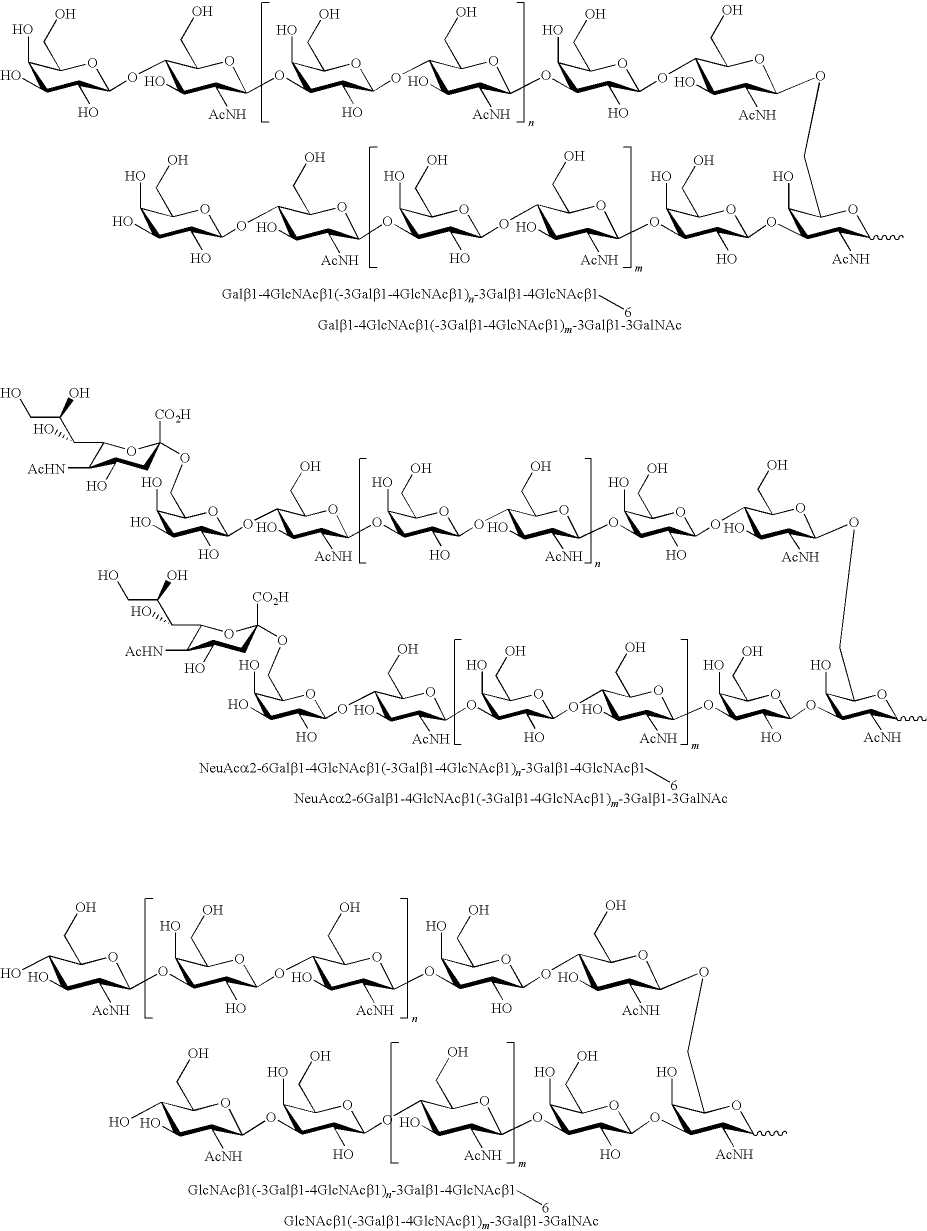

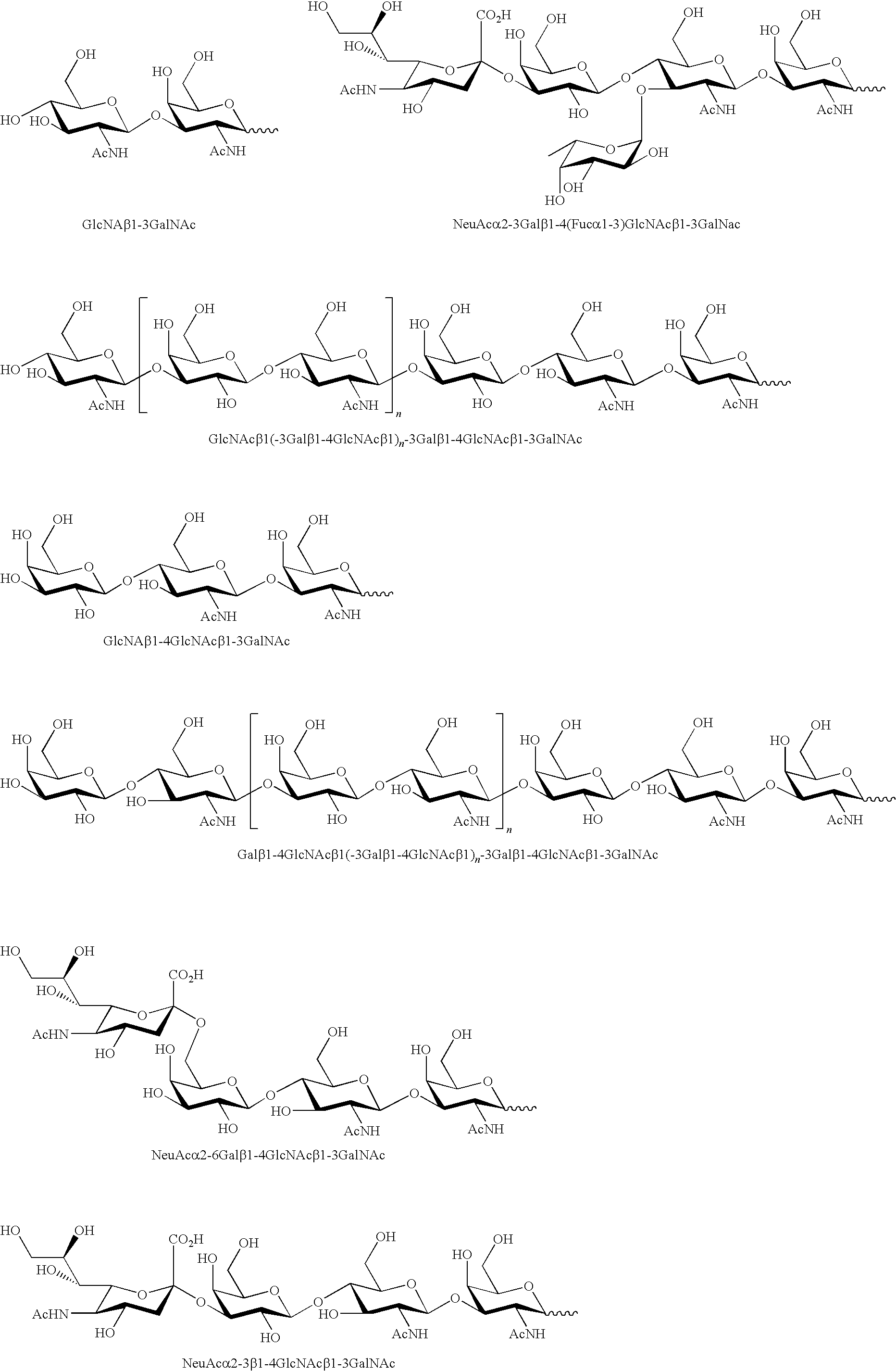

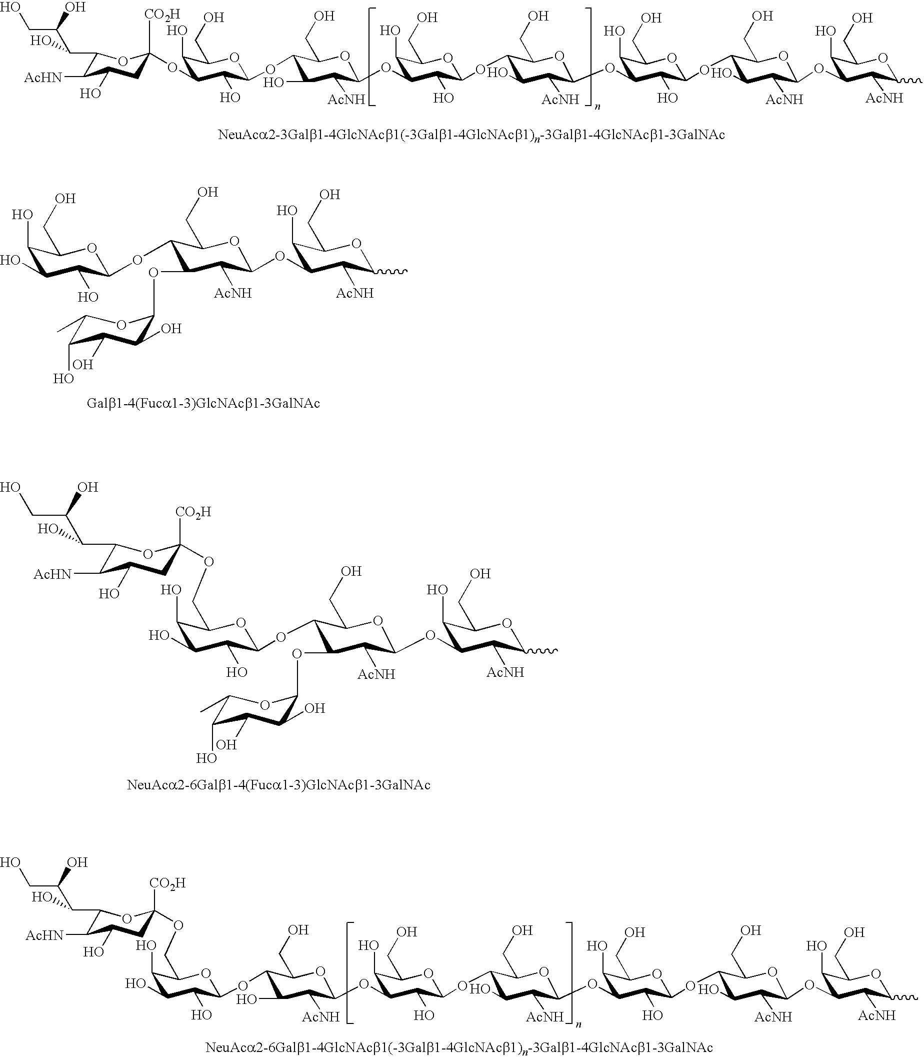

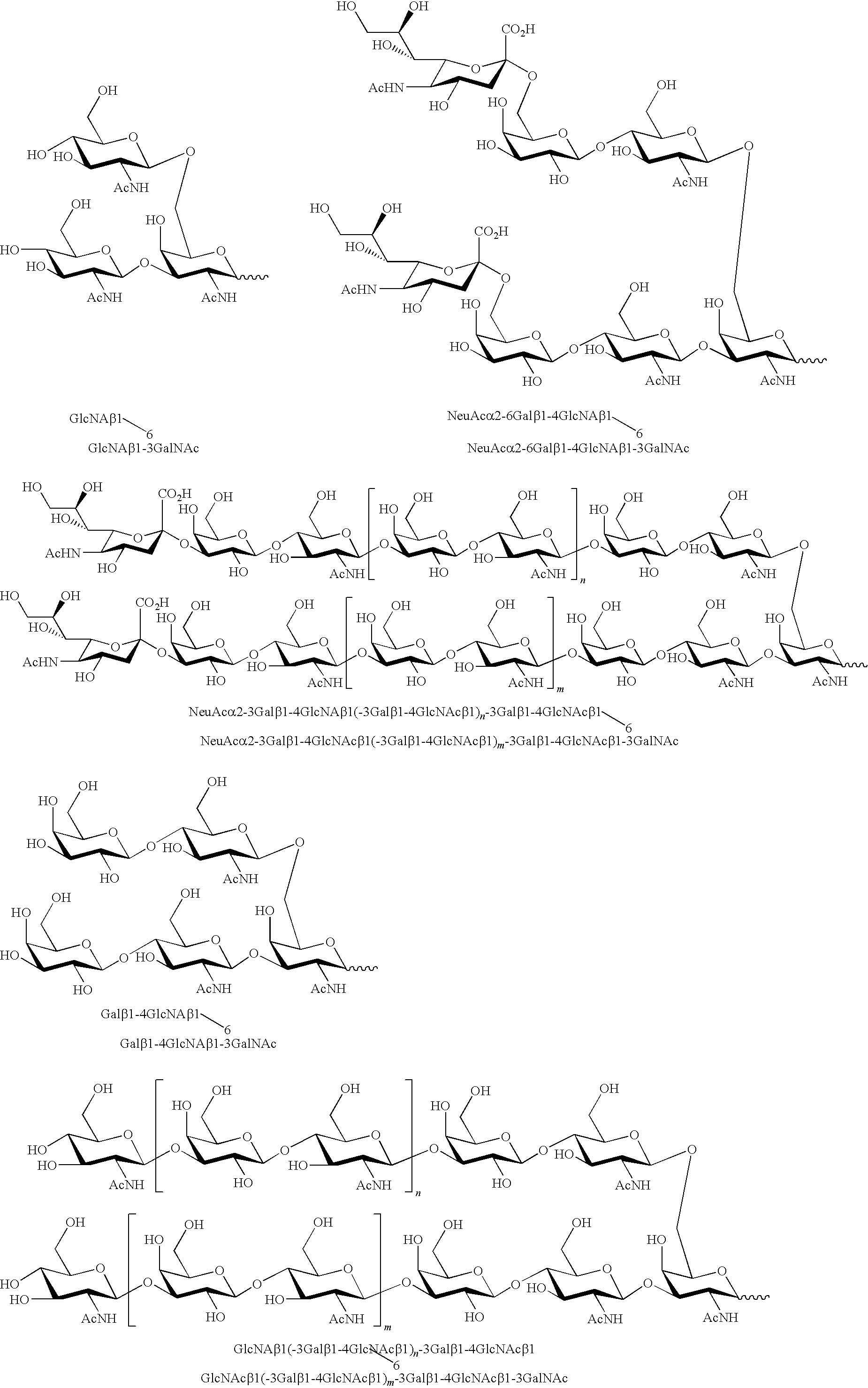

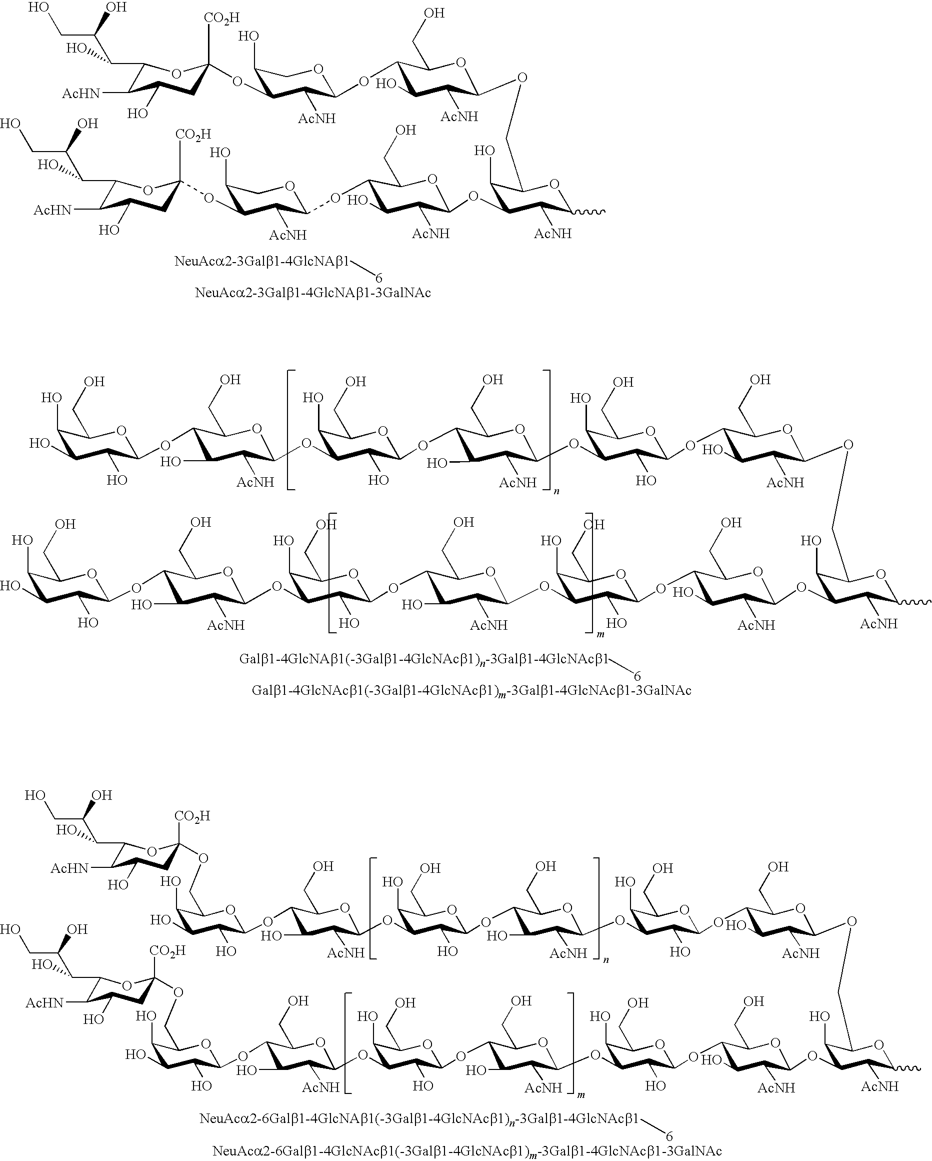

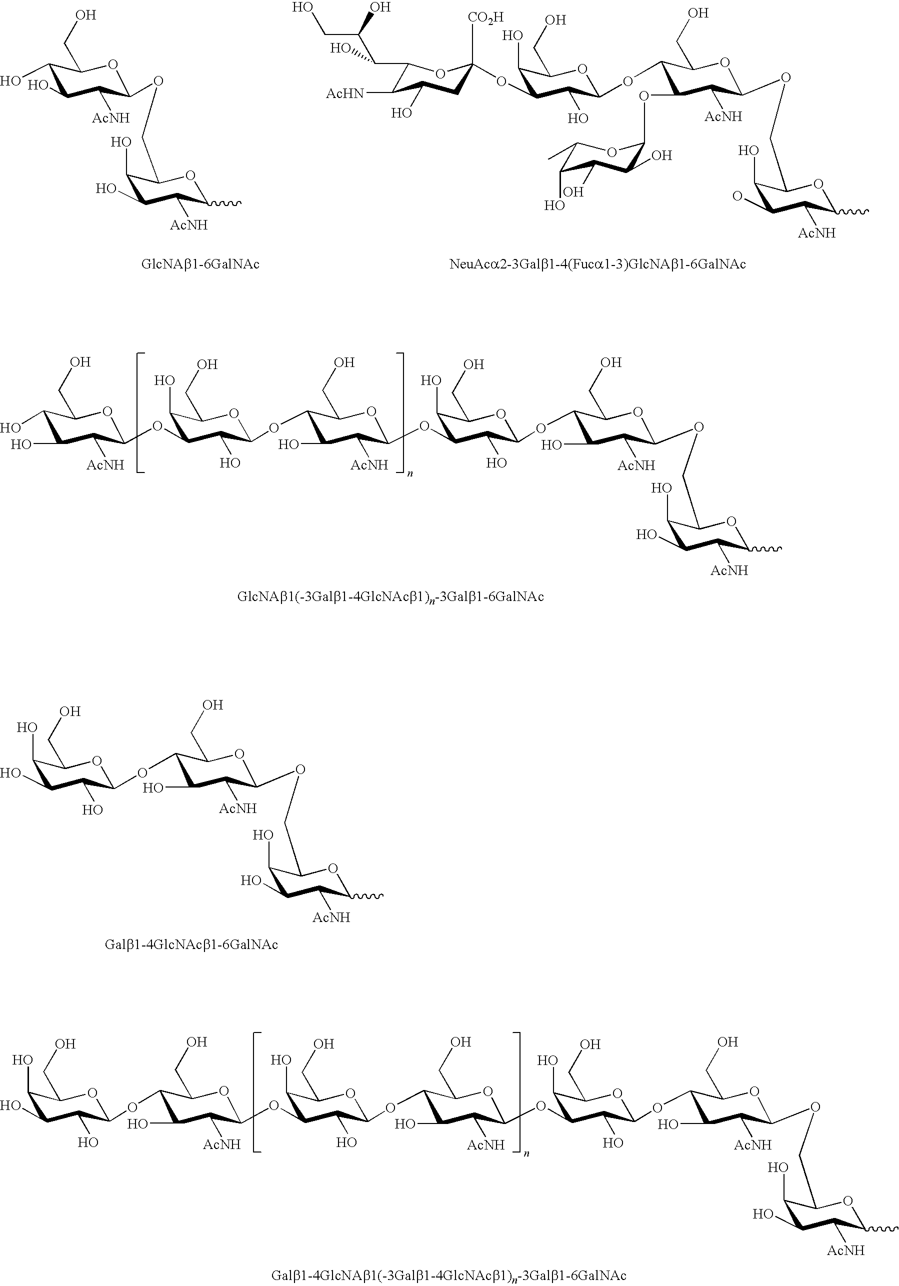

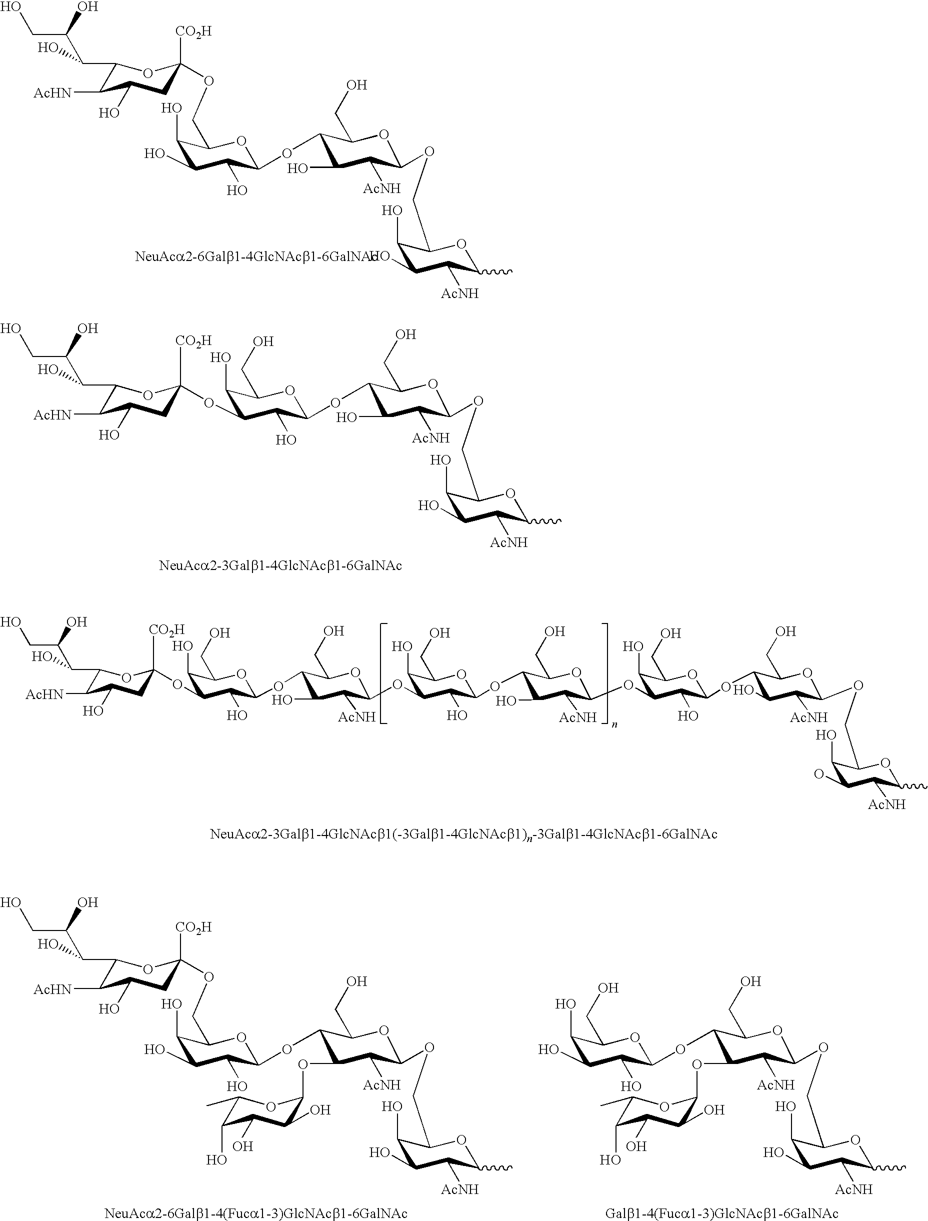

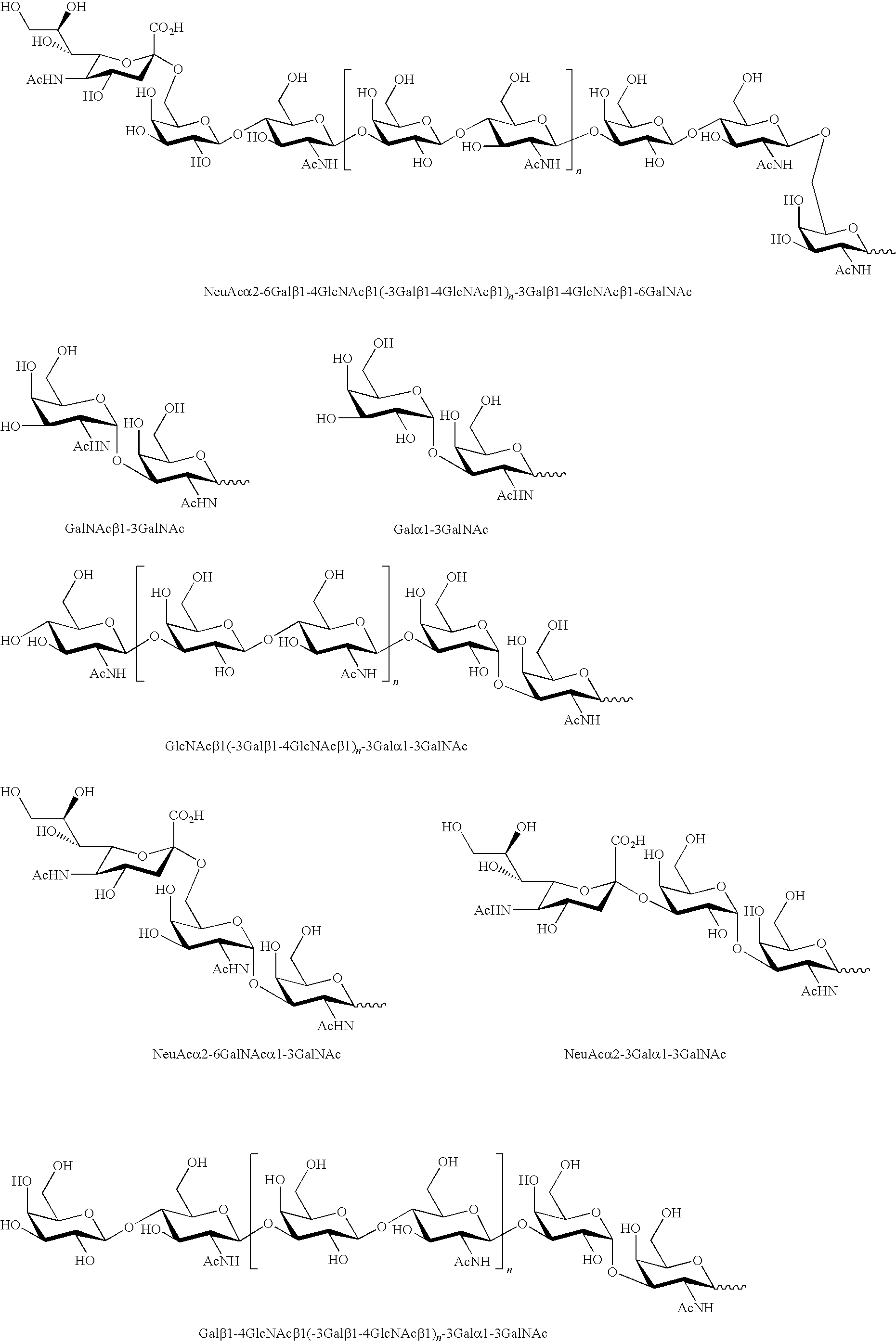

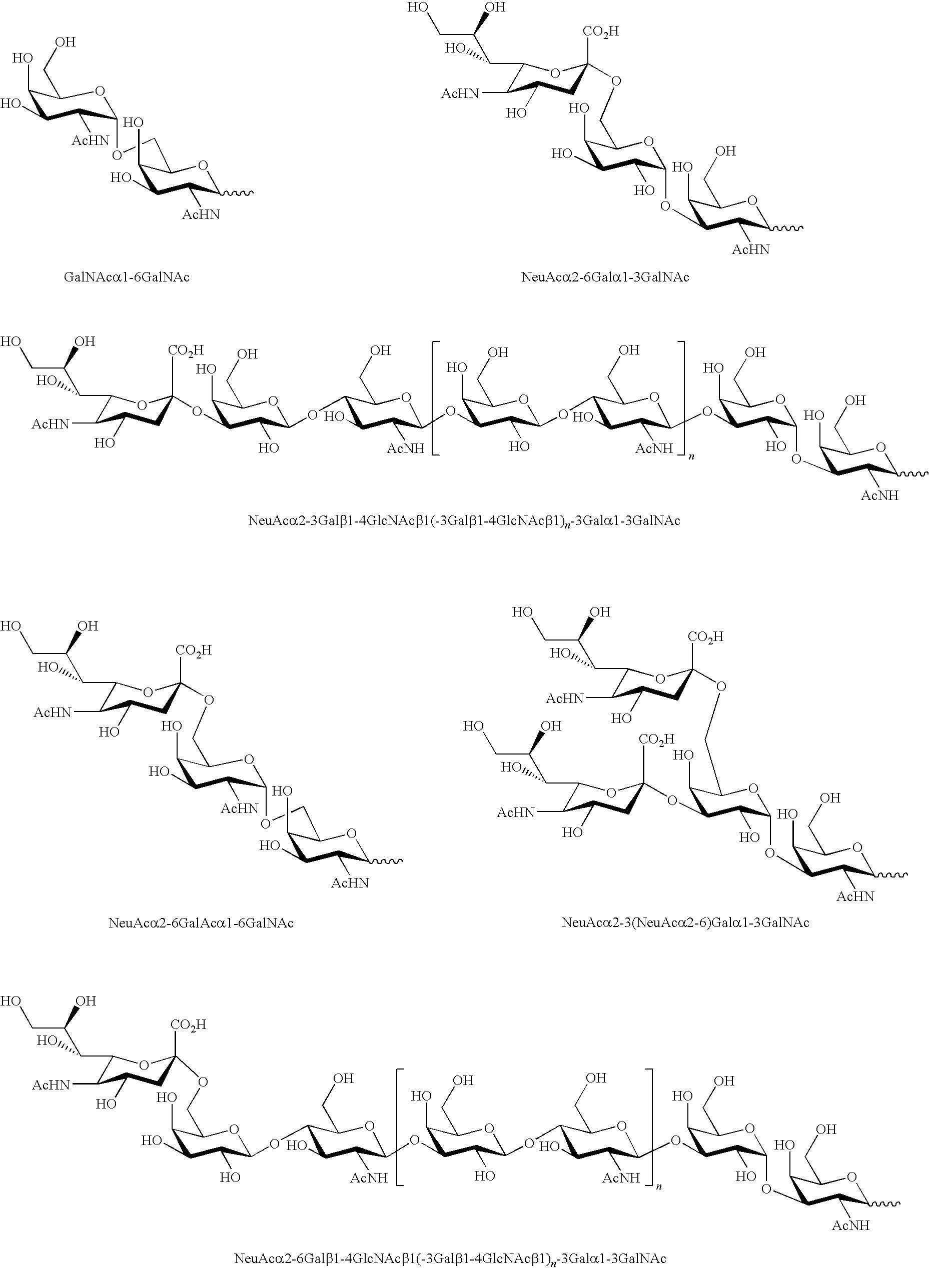

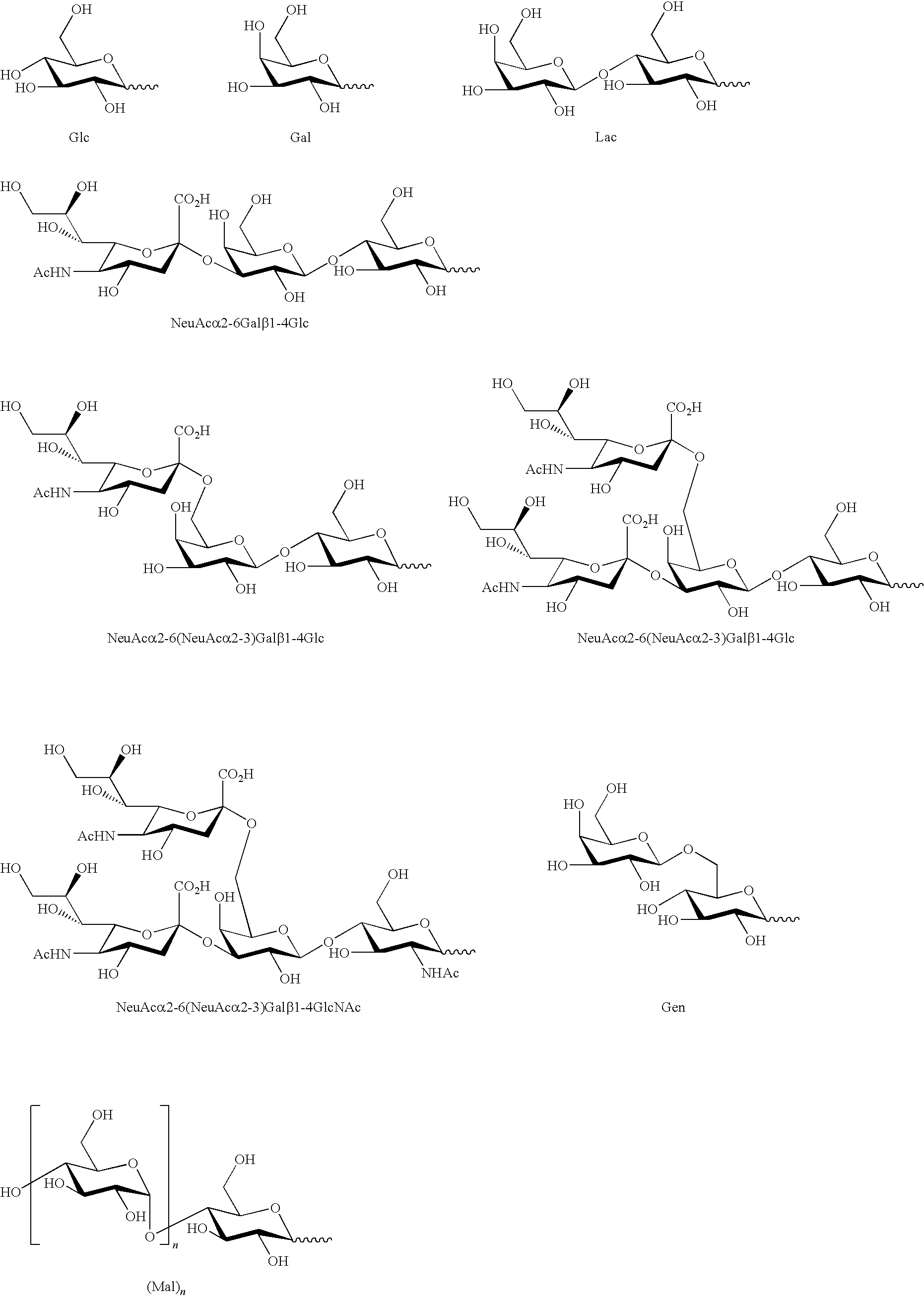

wherein n is an integer of 0-10, m is an integer of 0-10, and the symbols have the following meanings;

Fuc D-fucose

Gal D-galactose

GalNAc N-acetyl-D-galactosamine

Glc D-glucose

GlcNAc N-acetyl-D-glucosamine

Man D-mannose

NeuAc N-acetyl-D-neuraminic acid

Lac lactose

Gen gentiobiose

Examples of the especially preferred glycochain include

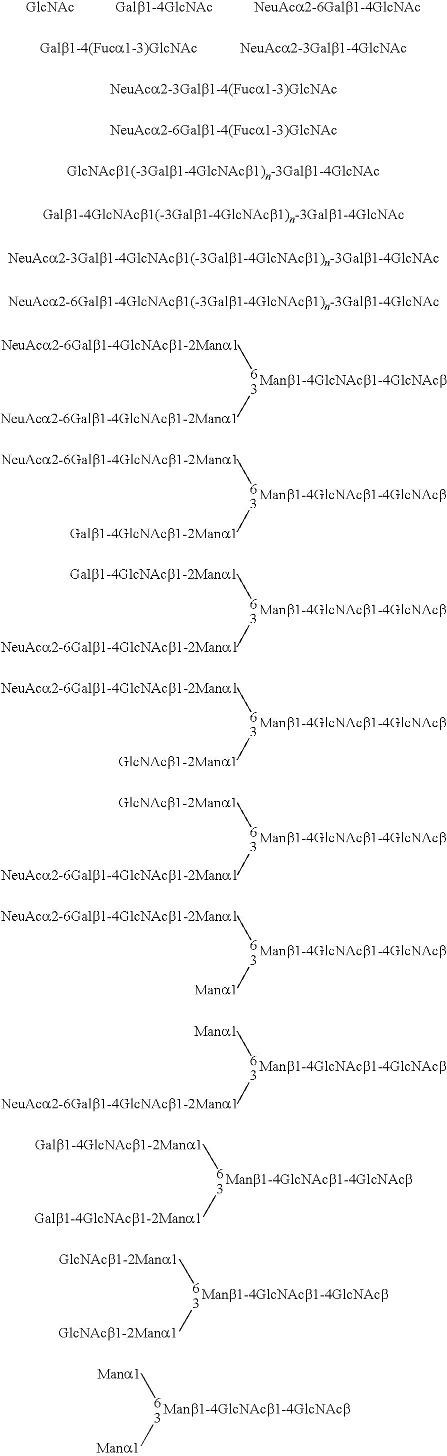

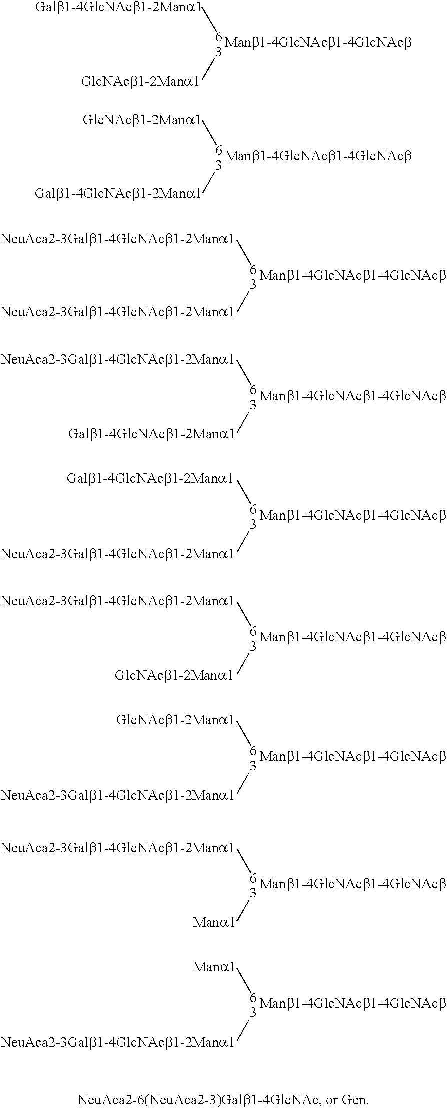

Examples of especially preferred glycosylated peptide include a derivative in which the especially preferred glycochain is attached to the preferred peptide of the formula (I), (II) and (III) described above.

In the specification, the term “degradative enzyme” means an enzyme involved in metabolism of GLP-1 related peptide such as DPP-IV, neutral endopeptidase and the like.

Peptide chain of GLP-1 related peptide can be appropriately synthesized by a solid phase peptide synthesis using Boc-method or Fmoc-method. Glycosilation may be carried out by the solid phase peptide synthesis using a monosaccharide of aminoacid such as Asn (GlcNAc) and subsequent additional modification of the glycochain, if necessary. A glycochain may be elongated by glycoltransferase etc.

Specifically, an Asn residue of the said peptide chain, the side chain of which is glycosylated (Asn-type), may be synthesized as followed;

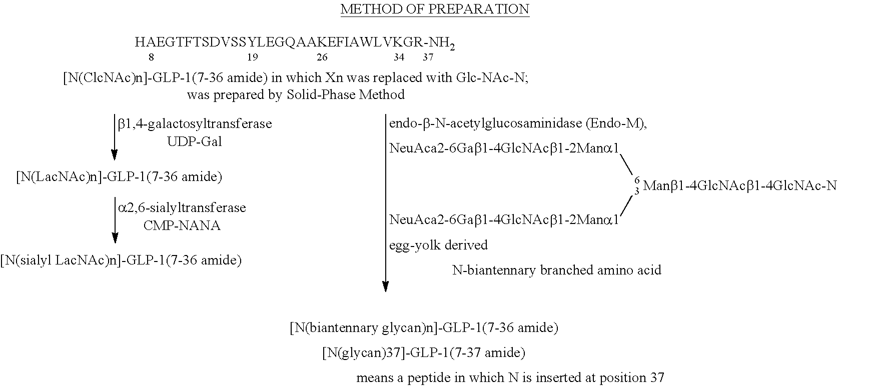

Also, a glycosylated derivative in which the side chain of Cys residue is glycosylated (Cys-type) may be synthesized by a general scheme:

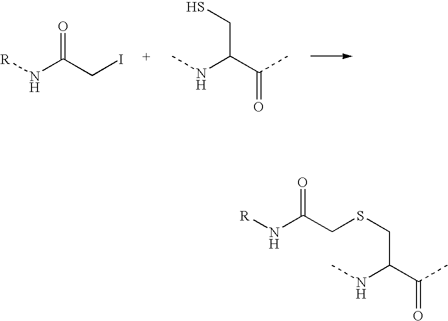

wherein the Cys-substituted peptide prepared by the solid phase peptide synthesis is coupled with the iodoacetyl derivative prepared by a chemical synthesis. In the scheme, the dotted line means the peptide chain of the GLP-1 related peptide and R is a glycochain.

The derivative containing the bitantennary-N-glycan is obtained by a method using endo-M enzyme or the reaction of the compound (3) of Reference Example and Cys-substituted peptide.

The derivative containing the biantennary-N-glycan dimer is obtained by the reaction of the compound (7) of Reference Example and Cys-substituted peptide.

The derivative containing the galactose trimer is obtained by the reaction of the compound (21) of Reference Example and Cys-substituted peptide.

### TEST EXAMPLES

Activities of the glycosylated peptide of the present invention to stimulate insulin secretion were evaluated by an agonistic activity (production of cAMP) and a receptor binding assay. Additionally, a prolonged activity of each glycosylated compound was evaluated by measuring a kinetic parameter of enzyme degradation caused by DPP-IV with GLP-1 derivatives, or testing a hypoglycemic activity with exendin 4 derivatives, which are resistant to DPP-IV.

1. Assay for Production of cAMP

CHO cells in which GLP-1 receptor was forced to express were seeded into 384-well plate in a concentration of 4000 cells/well and incubated for 48 hours. After being washed with the assay buffer (Hanks/20 mM HEPES, pH 7.4, 0.1% BSA) three times, the assay buffer (20 μL) was added to the cells and further 10 μL of a solution of GLP-1 derivative prepared with the assay buffer containing 0.1 mM IBMX and 0.2 mM R020-1724 (final concentration: 10⁻¹²-10⁻⁶M) was added. After stirring at room temperature for an hour, the cells were lysed with Triton X-100 (final concentration: 1%).

Quantity of cAMP was determined using cAMP Femtomolar Kit (CIS Bio International). The reaction solution (1 μL) was moved to a new 384-well plate and diluted by adding 9 μL of dilution buffer. Next, each 5 μL of cAMP-XL665 solution and anti-cAMP cryptate solution of the Kit was added, the mixture was incubated at room temperature for an hour, and time-resolved fluorescence was measured using RUBYstar (BMG LABTECH). The amount of formed cAMP was calculated based on the calibration curve of cAMP. A 100% activity was assigned to the maximum amount of cAMP produced by GLP-1, and a concentration to give a 50% activity was adopted as an ED₅₀ value of the tested compound.

### 2. Receptor Binding Assay

A membrane fraction (5 μL) prepared in the usual manner from CHO cells in which expression of GLP-1 receptor is forced was incubated with 62 pM[¹²⁵I]GLP-1 (7-36) (Perkin-Elmer), 25 mM HEPES, 5 mM MgCl, 1 mM CaCl₂, 0.25 mg/mL bacitracin, 0.1% BSA, and GLP-1 derivative (final conc. 10⁻¹¹ to 10⁻⁶M)(pH 7.4). After being incubated at room temperature for 2 hours, the solution was filtered through a unifilter 96GF/C plate (Perkin-Elmer) pretreated with 1% polyethylenimine containing 0.5% BSA, washed with 25 mM HEPES buffer solution (pH 7.4) containing 0.5% BSA, and radioactivity remained on the filter was measured by a gamma counter (Top Counter; Perkin-Elmer). An amount of binding under the presence of GLP-1 (1 μL) was considered as a non-specific binding. A concentration to give a 50% displacement of the specific binding of [¹²⁵I]GLP-1 (7-36) was adopted as an IC₅₀ value of each GLP-1 derivative.

### 3. Assay for Degradation Kinetics

GLP-1 analogue (20-500 mM) was incubated at 37° C. with 0.7 μg/mL recombinant human DPP-IV in 100 mMHEPES buffer containing 0.05% Tween 80 and 1 mM EDTA·2Na (pH 7.5)(60 μL). The reaction was carried out in a polypropylene tube having a volume of 1 mL immersed in a temperature controlled bath at 37° C. During the first 25 minutes, 7.0 μL of the reaction solution was sampled in every 5 minute, and concentration of a degradation product, a fragment peptide of the C terminus of the GLP-1 derivative produced by DPP-IV, was determined using HPLC. Develosil RPAQUEOUS-AR-3 (2.0×100 mm, Nomura Kagaku) was used as a column and the concentration was calculated based on the UV absorbance at 210 nm. The initial velocity of the degradation reaction was determined from a slope of the linear part obtained by plotting product concentration versus time. The initial velocity and concentration of the GLP-1 derivative were applied to Michaelis-Menten equation (1) and kinetics parameters, k_{cat} and K_{M} were determined as to each GLP-1 derivative.

$$V = \frac{{k}_{cat} \cdot E \cdot S}{{K}_{M} + S} \qquad ( 1 )$$

E: Enzyme concentration [M],
k_{cat}: Reaction rat constant [s⁻¹],
K_{M}: Michaelis Constant [M],
S: Substrate concentration [M],
V: Initial Velocity [Ms⁻¹]

### 4. Assay for Hypoglycemic Activity

Natural excendin-4 or its glycosylated derivative was administered (1 or 100 nmol/kg, s.c.) to a male BKS.Cg-+Leprdb/+Leprdb mouse of 12-17 weeks (CLEA Japan, Inc.) and blood glucose level was monitored using Glucocard (Arkray) after the administration. In a control group, only a solvent was administered. Animals were fasted from 1.5 hours before to the end of the experiments in a group of 1 nmol/kg administration while a group of 100 nmol/kg administration was under ad libitium fed condition, and blood samples were taken from tail vein.

Blood glucose level of each time was compared to that of a control group and it was judged “significant” if risk rate P is <0.05 by Tukey Test, and evaluation “A” means a compound wherein sustained period of the significant hypoglycemic activity is longer than that of the same dose of excendin-4, and evaluation “B” means a compound wherein it is the same as excendin-4. As for EX(1-28)NG and EX(1-28)NS6, the activity was compared to EX(1-28).

Results of cAMP-production assay and receptor-binding assay, and kinetic parameters useful for evaluating resistance against enzymatic degradation are shown in Table 1.

TABLE 1

| | In vitro activity | | resistance 1) | |
|---|---|---|---|---|
| | EC₅₀ 2) | IC₅₀ 3) | K_{M} | k_{cat}/K_{M} |
| compounds | [nM] | [nM] | [μM] | [10⁴/Ms] |
| GL | 0.11 | 0.41 | 27 | 14 |
| GL08N | 12 | 50 | 860 | 0.06 |
| GL08NG | >1000 | >100 | ND | ND |
| GL08NL | 284 | >100 | ND | ND |
| GL08NS6 | 583 | >100 | ND | ND |
| GL08NE1 | 200 | 420 | ND | ND |
| GL19N | 0.92 | 11 | 65 | 9.1 |
| GL19NG | 8.10 | 41 | 110 | 4.1 |
| GL19NL | 8.30 | 38 | 200 | 2.8 |
| GL19NS6 | 50 | >100 | 970 | 1.1 |
| GL19NE1 | 75 | 850 | 2500 | 0.23 |
| GL26N | 0.09 | 1.40 | 110 | 4.9 |
| GL26NG | 0.28 | 2.80 | 180 | 2.5 |
| GL26NL | 0.33 | 2.00 | 230 | 2.0 |
| GL26NS6 | 0.92 | 2.90 | 1000 | 0.92 |
| GL26NE1 | 0.80 | 11 | 2000 | 0.25 |
| GL34N | 0.11 | 1.00 | 91 | 4.9 |
| GL34NG | 0.17 | 0.85 | 160 | 2.1 |
| GL34NL | 0.11 | 0.76 | 150 | 2.5 |
| GL34NS6 | 0.16 | 0.70 | 760 | 1.2 |
| GL34NE1 | 0.16 | 2.70 | 520 | 0.44 |
| GL34CE1 | 0.72 | 3.5 | — | — |
| GL34CE2 | 3.40 | 20 | — | — |
| GL37N | 0.09 | 0.72 | 18 | 13 |
| GL37NG | 0.11 | 0.53 | 71 | 7.7 |
| GL37NL | 0.12 | 0.47 | 75 | 6.3 |
| GL37NS6 | 0.11 | 0.71 | 290 | 2.1 |
| GL37NS3 | 0.21 | 0.45 | — | — |
| GL37NS36 | 0.35 | 0.82 | — | — |
| GL37NE1 | 0.18 | 2.50 | 330 | 1.0 |
| GLSGSGS43NS6 | 0.11 | 1.00 | — | — |
| GL2634NS6 | 2.2 | 27 | — | — |
| GL2637NS6 | 1.0 | 21 | — | — |
| GL3437NG | 0.11 | 0.90 | 210 | 2.5 |
| GL3437NL | 0.11 | 0.92 | 250 | 1.8 |
| GL3437NS6 | 0.20 | 3.0 | 1400 | 0.21 |
| GL3437NS3 | 0.06 | 2.6 | — | — |
| GL3437NS36 | 0.45 | 4.1 | — | — |
| GL3437CE1 | 4.1 | 30 | >1000 | <0.10 |
| GL263437NS6 | 6.1 | 51 | — | — |

1) kinetic parameters of GLP-1 degradation by DPPIV:
ND means no degradation product
2) cAMP assay
3) receptor-binding assay

Results of cAMP-production assay and receptor-binding assay, and assay for sustained hypoglycemic in vivo activity are shown in Table 2-1 and 2-2.

TABLE 2-1

| | |
|---|---|
| In vitro activity [nM] | In vivo activity |

| compound | EC₅₀ | IC₅₀ | duration |
|---|---|---|---|
| EX | 0.55 | 0.1 | B |
| EX-01NG | >10 | 23 | — |
| EX01NG | >10 | 27 | — |
| EX02NG | >10 | >100 | — |
| EX03NG | >100 | 39 | — |
| EX04NG | >10 | 25 | — |
| EX05NG | >100 | 27 | — |
| EX06NG | >10 | >100 | — |
| EX07NG | >10 | 78 | — |
| EX08NG | >100 | 35 | — |
| EX09NG | >10 | 68 | — |
| EX10NG | >10 | 36 | — |
| EX11NG | >10 | 28 | — |
| EX12NG | 0.26 | 1.2 | — |
| EX13NG | 0.53 | 2.3 | — |
| EX14NG | >10 | 14 | — |
| EX15NG | >10 | >100 | — |
| EX16NG | 0.22 | 1.0 | — |
| EX17NG | 0.084 | 0.38 | — |
| EX18NG | >10 | 76 | — |
| EX19NG | >10 | >100 | — |
| EX20NG | 0.31 | 1.2 | — |
| EX21NG | 0.094 | 0.35 | — |
| EX22NG | >10 | >100 | — |
| EX23NG | >10 | >100 | — |
| EX24NG | 0.10 | 0.35 | — |
| EX25NG | 0.68 | 2.9 | — |
| EX26NG | >10 | >100 | — |
| EX27NG | 6.4 | 21 | — |
| EX28NG | 0.071 | 0.64 | B |
| EX29NG | 0.23 | 0.97 | — |
| EX30NG | 0.12 | 0.87 | — |
| EX31NG | 0.11 | 0.45 | — |
| EX32NG | 0.13 | 0.49 | — |
| EX33NG | 0.079 | 0.50 | — |
| EX34NG | 0.066 | 0.42 | — |
| EX35NG | 0.10 | 0.37 | — |
| EX36NG | 0.083 | 0.34 | — |
| EX37NG | 0.14 | 0.46 | — |
| EX38NG | 0.089 | 0.42 | — |
| EX39NG | 0.077 | 0.48 | — |
| EX40NG | 0.079 | 0.46 | — |
| EX12NS6 | 0.84 | 4.6 | — |
| EX13NS6 | 5.9 | 11 | — |
| EX16NS6 | 0.25 | 1.9 | — |
| EX17NS6 | 0.10 | 1.2 | B |
| EX20NS6 | 0.89 | 5.0 | B |
| EX21NS6 | 0.10 | 1.1 | A |
| EX24NS6 | 0.07 | 0.85 | B |

TABLE 2-2

| | |
|---|---|
| In vitro | |
| activity [nM] | In vivo activity |

| compound | EC₅₀ | IC₅₀ | duration |
|---|---|---|---|
| EX25NS6 | 1.7 | 19 | — |
| EX27NS6 | >10 | >100 | — |
| EX28NS6 | 0.09 | 0.87 | — |
| EX29NS6 | 0.18 | 1.3 | — |
| EX30NS6 | 0.11 | 1.6 | — |
| EX31NS6 | 0.11 | 1.0 | — |
| EX32NS6 | 0.11 | 1.5 | B |
| EX33NS6 | 0.084 | 0.99 | B |
| EX34NS6 | 0.083 | 0.96 | B |
| EX35NS6 | 0.085 | 0.72 | A |
| EX36NS6 | 0.085 | 0.72 | B |
| EX37NS6 | 0.14 | 0.99 | B |
| EX38NS6 | 0.10 | 0.84 | — |
| EX39NS6 | 0.094 | 0.86 | B |
| EX40NS6 | 0.05 | 0.86 | A |
| EX28NL | 0.039 | 0.52 | A |
| EX28NS3 | 0.029 | 0.49 | A |
| EX28NS36 | 0.036 | 0.72 | B |
| EX2840NG | 0.18 | 1 | — |
| EX2840NS6 | 0.06 | 1.3 | A |
| EX17212840NG | 0.13 | 1.5 | — |
| EX17213540NG | 0.11 | 1.3 | — |
| EX17283540NG | 0.061 | 0.73 | — |
| EX21283540NG | 0.092 | 1.1 | — |
| EX(1-28) | 0.074 | 0.99 | B |
| EX(1-28)28NG | 0.069 | 0.63 | A |
| EX(1-28)28NS6 | 0.099 | 1.1 | A |
| EX28CGlc | 0.065 | 0.4 | — |
| EX28CGal | 0.039 | 0.57 | B |
| EX28CLac | 0.024 | 0.41 | B |
| EX28CGen | 0.032 | 0.52 | B |
| EX28CSLac6 | 0.040 | 0.46 | B |
| EX28C7M | 0.047 | 0.62 | — |
| EX28CE1 | 0.091 | 3.2 | — |
| EX2840C7M | 0.085 | 0.76 | — |
| EX21283540C | 0.055 | 0.49 | B |
| EX21283540CGlc | 0.049 | 0.49 | — |
| EX21283540CGal | 0.049 | 0.53 | — |
| EX21283540CLac | 0.050 | 0.60 | — |
| EX21283540CGen | 0.052 | 0.65 | A |
| EX21283540CSLac6 | 0.26 | 3.7 | — |
| EX21283540C7M | 0.10 | 1.7 | — |
| EX28CJ3Gal | 0.034 | 0.41 | A |
| EX21283540CJ3Gal | 0.28 | 2.4 | — |

The result shows that a glycosylated peptide having glycochain at a position of 20 or later is preferable.

The present invention provides a pharmaceutical composition comprising a glycosylated GLP-1 related peptide or a pharmaceutically acceptable salt thereof, a dilutent and an excipient. The pharmaceutical composition is usually prepared in the common manner of the pharmaceutical field and preferably administered parenterally. Examples of especially preferable route of administration include intramuscular and subcutaneous administrations. Dosage of the glycosylated peptide a day is in the range of about 1 pg/kg body weight to about 1000 μg/kg body weight, but more or less dosage is also effective. The necessary dosage depends on condition of disease, body length, body weight, gender, age and/or past medical history of a patient.

The pharmaceutical composition of the present invention can be prepared according to the conventional method, for example, description of Remington: Pharmaceutical Science, 1985, or Remington: The Science and Practice of Pharmacy, 19th edition, 1995.

For example, a composition for infusion comprising the GLP-1 derivative of the present invention can be prepared to provide a requested final product by using the conventional technique in the pharmaceutical industry to dissolve and mix the ingredients appropriately.

In order to prepare the composition of the present invention, an effective ingredient (comprising a sort of glycosylated GLP-1 related peptide at least) is usually mixed with or diluted with an excipient. Before mixing with the other ingredients, the glycosylated GLP-1 related peptide may be crushed to a powder having a suitable diameter.

Examples of the excipient include lactose, dextrose, sucrose, trehalose, sorbitol, mannitol, starch, arabia gum, calcium silicate, microcrystalline cellulose, polyvinyl pyrrolidone, cellulose, water, syrup, methyl cellulose and the like. Further, a lubricant such as talc, magnesium stearate and mineral oil; a wetting agent, an emulsifying agent, a suspension agent, a preservative such as methyl or propylhydroxy benzoic acid; a sweetener and a flavouring agent.

Usually, a pharmaceutical composition is prepared in a dosage unit comprising an effective ingredient of about 50 μg to 100 mg, preferably about 1 mg to about 10 mg.

According to a procedure, a GLP-1 derivative may be dissolved in a somewhat smaller amount of water than that of the final volume of the composition. If necessary, an isotonic agent, a preservative agent and a buffer solution may be added, and the pH may be adjusted by adding an acid such as hydrochloric acid, or a base such as a sodium hydroxide aq. solution. Finally, the volume of solution is adjusted by adding water and a requested concentration of the ingredient will be provided. A composition for nasal administration comprising a specified peptide may be prepared according to the description of EP 272097 (Novo Nordisk A/S) or WO 93/18785.

In one aspect of the present invention, use of the GLP-1 derivative set forth above in manufacturing a pharmaceutical composition, especially the same for treating diabetes is provided.

In other aspect, a method for treating diabetes comprising an administration of the GLP-1 derivative set forth above.

### EXAMPLES

In the present specification, a peptide and its glycosylated derivative may be shown by abbreviations, and the nomenclature is described by example 1 and 2 below.

### Example 1

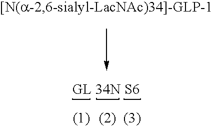

A part of (1) to (3) in the abbreviation “GL34NS6” means as follows;
(1) means a sort of peptide;

GL: GLP-1 (7-36) amide

GLSGSGSG: peptide of the above GL having an additional SGSGSG (amide) at the C terminus

EX: Excendin-4

EX(1-28): Excendin-4(1-28) amide

(2) means amino acid variation;

34N: means amino acid³⁴ was replaced with Asn,

28C: means amino acid²⁸ was replaced with Cys,

01N: means amino acid¹ was replaced with Asn,

3437N: means amino acid³⁴ and amino acid³⁷ were replaced with Asn respectively,

-1N: means Asn was added at the N-terminus.

(3) means a type of glycosilation as follows;

G: GlcNac

L: LacNac

S3: sialyl-α-2,3 LacNAc

S36: disialyl LacNAc

E1: branched N-glyco chain

E2: branched N-glyco chain aggregate

7M: maltoheptaose

SLac3: sialyl α-2,3 Lac

SLac6: sialyl α-2,6 Lac

J3Gal: Gal aggregate

### Example 2

### GL3437NS3

```
                                                    34 37
HAEGTFTSDVSSYLEGQAAKEFIAWLVKGR-NH2
                                                      ↑ ↑
```

“GL3437NS3” means a glycosylated peptide in which Asn sialylα-2,3 LacNAc was introduced in 34- and 37-position of GLP-1 (7-36) amide.

### Example 1

### Synthesis of 34-glycosylated GLP-1

### (1) Preparation of GL34N and GL34NG

GL34N and GL34NG were prepared by solid phase peptide synthesis using Boc method or Fmoc method, and the products were purified with HPLC having an ODS column and lyophilized.

### (2) Preparation of GL34NL

GL34NG(2 mM), UDP-Galactose (5 mM) and β-1,4-galactosyl transferase (0.2 U/mL, TOYOBO) were reacted in a solution (10 mM MnCl₂, 12.5 mM HEPES buffer pH 7.5) at 25° C. hor 2 hours. The reaction solution was concentrated by lyophilization and the product was purified with ODS column (Inertsil ODS-3 10×250 mm, GL Science) using 25 mM ammonium acetate-acetonitrile as an eluent.

### (3) Preparation of GL34NS6

GL34NG(2 mM), UDP-Galactose (5 mM) and β-1,4-galactosyl transferase (0.2 U/mL, TOYOBO) were reacted in a solution (10 mM MnCl₂, 12.5 mM HEPES buffer pH 7.5, 500 μl) at 25° C. for 2 hours. The reaction solution was concentrated by lyophilization and a solution of 10 mM CMP-sialic acid, 50 mU/mL α 2,6-sialyl transferase (TOYOBO) and 0.01% Triton X-100 was finally prepared by adding necessary agents. After the reacting volume was adjusted to 400 μl by adding water, the mixture was reacted at 37° C. for 23 hours. During the reaction, 100 mM CMP-sialic acid (40μl) was added. The product was purified with ODS column (Inertsil ODS-3 10×250 mm, GL Science) and 25 mM ammonium acetate-acetonitrile as an eluent.

### (4) Preparation of GL34NE1

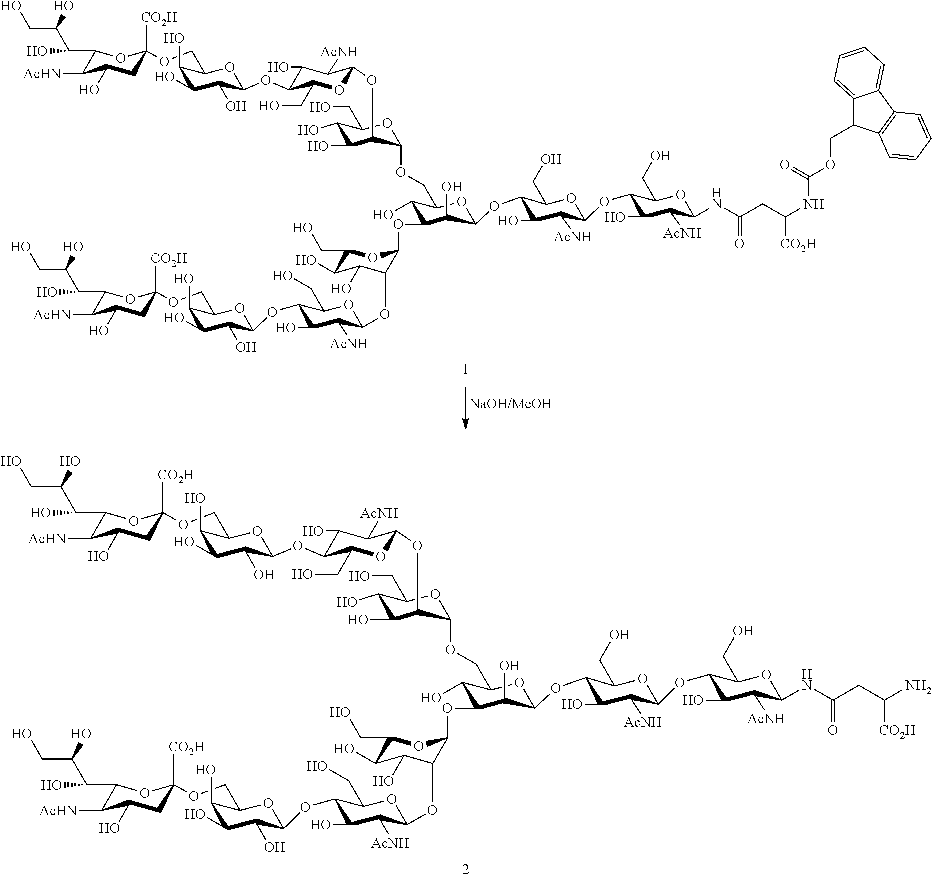

The compound (1)(150 mg, 58.6 μmol: Otsuka Cemical Co., Ltd.) was dissolved in methanol (60 mL) and a 1N sodium hydroxide aq. solution (1.8 mL) was added. After stirring at room temperature for 10 hours, the reaction was stopped by adding a 1N acetic acid aq, solution (3.6 mL). Methanol was evaporated, water and diethyl ether were added to the residue, and the aq. layer was washed with diethyl ether twice. A crude product of the compound (2) was obtained by lyophilization of the aq. layer, and then it was purified with a gel filtration chromatography (Sephadex G-50, mobile phase: water) to give the compound (2)(118 mg, 50.5 μmol), which was identified by MALDI-TOF-MS. MALDI-TOF-MS: [M(average)+Na]⁺=2360.0, (theoretical value: [(average)+Na]⁺=2361.1)

A reaction solution (60 mM potassium phosphate buffer pH 6.25) including GL34NL(10 mM), the compound (2)(75 mM) and endo-β-N-acetylglucosaminidase (60 mU/mL, Tokyo Chemical Industry Co., Ltd.) was reacted at 37° C. for 2 hours, then the reaction was stopped by adding an equal amount of 8M guanidine hydrochloride solution and the product was purified with a reversed phase HPLC.

### Example 2

### Synthesis of 37-glycosylated GLP-1

### (1) Preparation of GL37N and GL37NG

GL37N and GL37NG were prepared by solid phase peptide synthesis using Boc method or Fmoc method, and the products were purified with HPLC having an ODS column and lyophilized.

### (2) Preparation of GL37NL

GL37NG(1 mM), UDP-Galactose (3 mM) and β-1,4-galactosyl transferase (0.2 U/mL, TOYOBO) were reacted in a solution (10 mM MnCl₂, 12.5 mM HEPES buffer pH 7.5 2 mL) at 25° C. hor 2 hours. The reaction solution was concentrated by lyophilization and the product was purified with a reversed phase HPLC.

### (3) Preparation of GL37NS6

GL37NG(1 mM), UDP-Galactose (3 mM) and β-1,4-galactosyl transferase (0.2 U/mL, TOYOBO) were reacted in a reaction solution (10 mM MnCl₂, 12.5 mM HEPES buffer pH 7.5, 1 mL) at 25° C. for 2 hours. Then 100 mM CMP-sialic acid (50 μl), 1 U/mL α 2,6-sialyl transferase (TOYOBO)(50 μl) and 1% Triton X-100 (10 μl) were added and the mixture was reacted at 25° C. for 26 hours. Further it was reacted at 37° C. for 39 hours. During the reaction, 100 mM CMP-sialic acid (50 μl) and 1 U/mL α 2,6-sialyl transferase (TOYOBO)(25 μl) were added. The product was concentrated with lyophilization and purified with a reversed phase HPLC.

### (4) Preparation of GL37NS3

GL37NG(2 mM), UDP-Galactose (5 mM) and β 1,4-galactosyl transferase (0.2 U/mL, TOYOBO) were reacted in a reaction solution (5 mM MnCl₂, 12.5 mM HEPES buffer pH 7.5, 1.4 mL) at 25° C. for 2 hours, purified with a reversed phase HPLC and lyophilized. The GL37NL obtained in the above procedure was dissolved again in distilled water, and a reaction solution (50 mM HEPES buffer pH 7.5, 0.01 Triton X-100, 2 mL) containing GL37NL(1 mM), CMP-sialic acid (5 mM) and α 2,6-sialyl transferase (50 mU/mL, CALBIOCHEM) was prepared and it was reacted at 37° C. for 0.5 hours. Then it was concentrated with lyophilization and purified with a reversed phase HPLC.

### (5) Preparation of GL37NS36

A reaction solution (5 mM MnCl₂, 20 mM cacodylic acid buffer pH 7.0, 2 mL) containing GL37NS3 (1 mM), CMP-sialic acid (5 mM) and α 2,6-sialyl transferase (JAPAN TOBACCO INC)(50mU/mL) was reacted at 30° C. for 16 hours and the product was purified with a reversed phase HPLC and lyophilized.

(6) A reaction solution (60 mM potassium phosphate buffer pH 6.25) containing GL37NL(10 mM), the compound (2)(75 mM) and endo-β-N-acetylglucosaminidase (60 mU/mL, Tokyo Chemical Industry Co., Ltd.) was reacted at 37° C. for 2 hours, then the reaction was stopped by adding an equal amount of 8M guanidine hydrochloride solution and the product was purified with a reversed phase HPLC.

### Example 3

### Preparation of Other Glycosylated GLP-1

The following glycosylated GLP-1's were prepared in the same manner as Examples 1 and 2

GL08N, GL08NG, GL08NL, GL08NS6, GL08NE1, GL19N, GL19NG, GL19NL, GL19NS6, GL19NE1, GL26N, GL26NG, GL26NL, GL26NS6, GL26NE1, GLSGSGSG43NG, GLSGSGSG43NL and GLSGSGSG43NS6.

MS spectrum data were shown in Tables 3.

TABLE 3

| | | theoretical | measured | ionization |
|---|---|---|---|---|
| Example | cmpound | figure (MW) | value (MW) | method |
| 1(1) | GL34N | 3283.6 | 3283.8 | MALDI |
| 1(1) | GL34NG | 3486.8 | 3486.8 | MALDI |
| 1(2) | GL34NL | 3649.0 | 3649.0 | MALDI |
| 1(3) | GL34NS6 | 3940.2 | 3940.1 | MALDI |
| 1(4) | GL34NE1 | 5489.7 | 5489.7 | MALDI |
| 2(1) | GL37N | 3411.8 | 3411.8 | MALDI |
| 2(1) | GL37NG | 3615.0 | 3615.1 | MALDI |
| 2(2) | GL37NL | 3777.1 | 3777.7 | MALDI |
| 2(3) | GL37NS6 | 4068.4 | 4068.4 | MALDI |
| 2(4) | GL37NS3 | 4068.4 | 4067.8 | ESI |
| 2(5) | GL37NS36 | 4359.7 | 4359.0 | ESI |
| 2(6) | GL37NE1 | 5617.8 | 5617.9 | MALDI |
| 3 | GL08N | 3340.7 | 3340.3 | MALDI |
| 3 | GL08NG | 3543.9 | 3544.0 | MALDI |
| 3 | GL08NL | 3706.1 | 3706.2 | MALDI |
| 3 | GL08NS6 | 3997.3 | 3997.9 | MALDI |
| 3 | GL08NE1 | 5546.7 | 5546.4 | MALDI |
| 3 | GL19N | 3248.6 | 3248.4 | MALDI |
| 3 | GL19NG | 3451.8 | 3451.7 | MALDI |
| 3 | GL19NL | 3614.0 | 3613.5 | MALDI |
| 3 | GL19NS6 | 3905.2 | 3905.1 | MALDI |
| 3 | GL19NE1 | 5454.6 | 5454.9 | MALDI |
| 3 | GL26N | 3283.6 | 3283.5 | MALDI |
| 3 | GL26NG | 3486.8 | 3486.9 | MALDI |
| 3 | GL26NL | 3649.0 | 3648.9 | MALDI |
| 3 | GL26NS6 | 3940.2 | 3939.8 | MALDI |
| 3 | GL26NE1 | 5489.7 | 5489.0 | MALDI |
| 3 | GLSGSGSG43NG | 4047.4 | 4045.6 | MALDI |
| 3 | GLSGSGSG43NL | 4209.5 | 4208.5 | MALDI |
| 3 | GLSGSGSG43NS6 | 4500.8 | 4501.2 | MALDI |

ESI: Electrospray ionization Method
MALDI: Matrix Assisted Laser Desorption/Ionization Method

### Example 4

### Synthesis of 34- and 37-glycosylated GLP-1

### (1) Preparation of GL3437NG

GL3437NG was prepared by solid phase peptide synthesis using Boc method or Fmoc method, and the products were purified with HPLC having an ODS column and lyophilized.

### (2) Preparation of GL3437NL

GL3437NG(2 mM), UDP-Galactose (6 mM), β 1,4-galactosyl transferase (0.2 U/mL, TOYOBO) and MnCl₂ (10 mM) were reacted in a solution (25 mM HEPES buffer pH 7.5) at 25° C. hor 16 hours and the product was purified with ODS column (Inertsil ODS-3 10×250 mm, GL Science) and 25 mM ammonium acetate-acetonitrile as an eluent.
(3) Preparation of GL3437NS6

A reaction solution (25 mM HEPES buffer pH 7.5) containing GL3437NL(1 mM), CMP-sialic acid (10 mM), 50 mU/mL α 2,6-sialyl transferase (0.1 U/mL, TOYOBO) and 0.01% Triton X-100 was reacted at 37° C. for 14 hours. The product was purified with ODS column (Inertsil ODS-3 10×250 mm, GL Science) and 25 mM ammonium acetate-acetonitrile as an eluent.

### (4) Preparation of GL3437NS3

A reaction solution (50 mM HEPES buffer pH 7.5) containing GL3437NL(1 mM), CMP-sialic acid (10 mM), α 2,3-sialyl transferase (0.05 U/mL, Calbiochem) and 0.01% Triton X-100 was reacted at 37° C. for 3.5 hours. Then, the product was purified with Inertsil ODS-3 10×250 mm (GL Science) using 25 mM ammonium acetate-acetonitrile as an eluent.

### (5) Preparation of GL3437NS36

A reaction solution (5 mM MnCl₂, 20 mM cacodylic acid buffer pH 7.0, 0.44 mL) containing GL3437NS3 (1 mM), CMP-sialic acid (10 mM) and α 2,6-sialyl transferase (50 mU/mL, JAPAN TOBACCO INC) was reacted at 30° C. for 16 hours and the product was purified with a reversed phase HPLC and lyophilized.

### Example 5

### Preparation of Other Glycosylated GLP-1

The following glycosylated peptides were prepared in the same manner as Examples 4;

GL2634NG, GL2634NL, GL2634NS6, GL2637NG, GL2637NL, GL2637NS6, GL263437NG, GL263437NL and GL263437NS6.

### Example 6

### Synthesis of Glycosylated Peptide by Modification of a Side Chain Thiol Group in Cys-Variational GLP-1

### (1) Preparation of GL34C and GL3437C

GL34C and GL3437C were prepared by solid phase peptide synthesis using Boc method or Fmoc method, and the products were purified with HPLC having an ODS column and lyophilized.

### (2) Preparation of GL34CE1

A reaction solution (100 mM phosphate buffer pH 8.0) containing GL34C(0.5 mM) and the compound (3)(1 mM, 1.2 mL) was reacted at 37° C. for 24 hours. The mixture was purified with a reversed phase HPLC(Inertsil ODS-3 10×250 mm, GL Science) using 0.1% TFA aq. solution and 0.1% TFA acetonitrile as an eluent to give GL34CE1.

### (3) Preparation of GL3437CE1

A reaction solution (100 mM phosphate buffer pH 8.0) containing GL3437C(0.5 mM) and the compound (3)(1.5 mM, 0.85 mL) was reacted. An aq. solution of the compound (3)(5 mM, 0.1 mL) was added 10 hours later and the mixture was further reacted for 13 hours. The mixture was purified with a reversed phase HPLC(Inertsil ODS-3 10×250 mm, GL Science) using 0.1% TFA aq. solution and 0.1% TFA acetonitrile as an eluent to give GL3437CE1.

### (4) Preparation of GL34CE2

A reaction solution (100 mM phosphate buffer pH 8.0) containing GL34C(2 mM) and the compound (7)(2.5 mM, 0.2 mL) was reacted at 37° C. for 25.5 hours. The mixture was purified with a reversed phase HPLC(Inertsil ODS-3 10×250 mm, GL Science) using 0.1% TFA aq. solution and 0.1% TFA acetonitrile as an eluent to give GL34CE2.

### (5) Preparation of GL3437CE2

A reaction solution (100 mM phosphate buffer pH 8.0) containing GL3437C(0.25 mM) and the compound (7)(1 mM, 30 μl) was reacted at 37° C. and the formation of GL3437CE2 was confirmed by MALDI-TOF-MS.

MS spectrum data of the compounds obtained in Examples 4-6 were shown in Tables 4.

TABLE 4

| | | theoretical | measured value | ionization |
|---|---|---|---|---|
| Example | compound | figure (MW) | (MW) | method |
| 4(1) | GL3437NG | 3804.1 | 3803.9 | ESI |
| 4(2) | GL3437NL | 4128.4 | 4129.3 | ESI |
| 4(3) | GL3437NS6 | 4710.9 | 4710.7 | ESI |
| 4(4) | GL3437NS3 | 4710.9 | 4710.7 | ESI |
| 4(5) | GL3437NS36 | 5293.5 | 5291.7 | ESI |
| 5 | GL2634NG | 3676.0 | 3675.6 | ESI |
| 5 | GL2634NL | 4000.2 | 4000.0 | ESI |
| 5 | GL2634NS6 | 4582.8 | 4582.2 | ESI |
| 5 | GL2637NG | 3804.1 | 3803.8 | ESI |
| 5 | GL2637NL | 4128.4 | 4127.9 | ESI |
| 5 | GL2637NS6 | 4710.9 | 4711.1 | ESI |
| 5 | GL263437NG | 3993.3 | 3993.0 | ESI |
| 5 | GL263437NL | 4479.7 | 4479.7 | ESI |
| 5 | GL263437NS6 | 5353.5 | 5352.3 | ESI |
| 6(1) | GL3437C | 3375.8 | 3375.5 | ESI |
| 6(1) | GL34C | 3272.7 | 3272.3 | ESI |
| 6(2) | GL34CE1 | 5650.8 | 5649.6 | ESI |
| 6(3) | GL3437CE1 | 8132.1 | 8129.2 | ESI |
| 6(4) | GL34CE2 | 8100.1 | 8095.5 | ESI |
| 6(5) | GL3437CE2 | 13030.4 | 13035.2 | MALDI |

### Example 7

### Synthesis of 28-glycosylated excendin-4

### (1) Preparation of EX28NG

EX28NG was prepared by solid phase peptide synthesis using Boc method or Fmoc method, and the products were purified with HPLC having an ODS column and lyophilized.

### (2) Preparation of EX28NL

A reaction solution (10 mM MnCl₂, 25 mM HEPES buffer pH 7.5) containing EX28NG(2 mM), UDP-Galactose (5 mM), β 1,4-galactosyl transferase (0.2 U/mL, TOYOBO) was reacted at 25° C. for 3 hours and the product was purified with C30 column (Develosil RPAQUEOUS AR-5 10×250 mm, NOMURA CHEMICAL CO., LDP.) using 25 mM ammonium acetate-acetonitrile as an eluent.

### (3) Preparation of EX28NS6

A reaction solution (10 mM MnCl₂, 12.5 mM HEPES buffer pH 7.5) containing EX28NG(1 mM), UDP-Galactose (5 mM) and β 1,4-galactosyl transferase (0.1 U/mL, TOYOBO) was reacted at 25° C. for 2 hours. One tenth amount of 100 mM CMP-sialic acid and one tenth amount of 1 U/mL α 2,6-sialyl transferase (TOYOBO) were added and the solution was reacted at 37° C. for 19 hours, and the product was purified with ODS column (Inertsil, ODS-3 10×250 mm, GL Science).

### (4) Preparation of EX28NS3

A reaction solution (50 mM HEPES buffer pH 7.5) containing EX28NL(1 mM), CMP-sialic acid (10 mM), α 2,3-sialyl transferase (0.05 U/mL, Calbiochem) and 0.01% Triton X-100 was reacted at 37° C. for 17 hours. Then, the product was purified with C30 column RPAQUEOUS AR-5 10×250 mm (NOMURA CHEMICAL CO., LDP.) using 25 mM ammonium acetate-acetonitrile as an eluent.

### (5) Preparation of EX28NS36

A reaction solution (50 mM HEPES buffer pH 7.5) containing EX28NS3 (1 mM), CMP-sialic acid (20 mM) and α 2,6-sialyl transferase (0.2 U/mL, JAPAN TOBACCO INC) was reacted at 30° C. for 70 hours and the product was purified with C30 column RPAQUEOUS AR-5 10×250 mm (NOMURA CHEMICAL CO., LDP.).

### Example 8

### Preparation of Other Glycosylated Excendin-4

The following glycosylated peptides were prepared in the same manner as Examples 7;

EX-1NG, EX01NG, EX02NG, EX03NG, EX04NG, EX05NG, EX06NG, EX07NG, EX08NG, EX09NG, EX10NG, EX11NG;

EX12NG, EX12NL, EX12NS6;

EX13NG, EX13NL, EX13NS6;

EX14NG, EX15NG,

EX16NG, EX16NL, EX16NS6;

EX17NG, EX17NL, EX17NS6;

EX18NG, EX19NG,

EX20NG, EX20NL, EX20NS6;

EX21NG, EX21NL, EX21NS6;

EX22NG, EX23NG,

EX24NG, EX24NL, EX24NS6;

EX25NG, EX25NL, EX25NS6;

EX26NG,

EX27NG, EX27NL, EX27NS6;

EX29NG, EX29NL, EX29NS6;

EX30NG, EX30NL, EX30NS6;

EX31NG, EX31NL, EX31NS6;

EX32NG, EX32NL, EX32NS6;

EX33NG, EX33NL, EX33NS6;

EX34NG, EX34NL, EX34NS6;

EX35NG, EX35NL, EX35NS6;

EX36NG, EX36NL, EX36NS6;

EX37NG, EX37NL, EX37NS6;

EX38NG, EX38NL, EX38NS6;

EX39NG, EX39NL, EX39NS6;

EX40NG, EX40NL, EX40NS6;

EX2840NG, EX2840NL, EX2840NS6;

EX17212840NG, EX17213540NG, EX17283540NG, EX21283540NG;

EX(1-28),

EX(1-28)28NG, EX(1-28)28NL, EX(1-28)28NS6.

### Example 9

### Synthesis of Glycosylated Peptide by Modification of a Side Chain Thiol Group in Cys-Variational Excendin-4

### (1) Preparation of EX28C and EX21283540C

EX28C and EX21283540C were prepared by solid phase peptide synthesis using Boc method or Fmoc method, and the products were purified with HPLC having an ODS column and lyophilized.

### (2) Preparation of EX28CE1

A reaction solution (100 mM phosphate buffer pH 8.0) containing EX28C(1 mM) and the compound (3)(2 mM, 0.5 mL) was reacted at 37° C. for 23 hours. The mixture was purified with a reversed phase HPLC(Inertsil ODS-3 10×250 mm, GL Science) to give EX28CE1.

### (3) Preparation of EX28CJ3Gal

A reaction solution (100 mM phosphate buffer pH 8.0) containing EX28C(1 mM) and the compound (21)(2.7 mM, 0.36 mL) was reacted at 37° C. for 3.5 hours. The mixture was purified with a reversed phase HPLC(Inertsil ODS-3 10×250 mm, GL Science) using 0.1% TFA aq. solution and 0.1% TFA acetonitrile as an eluent to give EX28CJ3Gal.

### (4) Preparation of EX21283540CJ3Gal

A reaction solution (100 mM phosphate buffer pH 8.0) containing EX21283540C(1 mM) and the compound (21)(8 mM, 0.23 mL) was reacted at 37° C. EX28C(0.5 mg) was added 2 hours later and the solution was further reacted for an hour. Then the mixture was purified with a reversed phase HPLC(Inertsil ODS-3 10×250 mm, GL Science) using 0.1% TFA aq. solution and 0.1% TFA acetonitrile as an eluent to give EX21283540CJ3Gal.

### (5) Preparation of EX28C7M

A reaction solution (100 mM phosphate buffer pH 8.0) containing EX28C(1 mM) and the compound (9)(4 mM, 0.40 mL) was reacted at 37° C. for 3.5 hours and purified with C30 column RPAQUEOUS AR-5 10×250 mm (NOMURA CHEMICAL CO., LDP.) to give EX28C7M.

### (6) Preparation of EX21283540C7M

A reaction solution (100 mM phosphate buffer pH 8.0) containing EX21283540C(1 mM) and the compound (9)(8 mM, 0.37 mL) was reacted at 37° C. for 1.5 hours and purified with C30 column RPAQUEOUS AR-5 10×250 mm (NOMURA CHEMICAL CO., LDP.) to give EX21283540C7M.

(7) Preparation of other glycosylated peptides

The following glycosylated peptides were prepared in the same manner as (5) and (6) above.

EX28CGlc, EX28CGal, EX28CLac, EX28CGen, EX2840C7M;

EX21283540CGlc, EX21283540CGal, EX21283540CLac, EX21283540Cgen.

### (8) Preparation of EX28CSLac6

A reaction solution (50 mM Tris-HCl buffer pH 7.5) containing EX28CLac (0.5 mM), CMP-sialic acid (10 mM) and α 2,6-sialyl transferase (0.1 U/mL, JAPAN TOBACCO INC) and 0.01% Triton X-100 was reacted at 16° C. for 70 hours and the product was purified with ODS column Inertsil ODS-3 110×250 mm (GL Science) using 25 mM ammonium acetate and acetonitrile as an eluent.

### (9) Preparation of EX21283540CSLac6

A reaction solution (50 mM Tris-HCl buffer pH 7.5) containing EX21283540CLac (0.5 mM), CMP-sialic acid (10 mM) and α 2,6-sialyl transferase (0.1 U/mL, JAPAN TOBACCO INC) and 0.01% Triton X-100 was reacted at 30° C. for 16 hours and the product was purified with ODS column Inertsil ODS-3 10×250 mm (GL Science) using 25 mM ammonium acetate and acetonitrile as an eluent.

MS spectrum data of the compounds obtained in Examples 6-9 were shown in Tables 5.

| | | theoretical | measured | ionization |
|---|---|---|---|---|
| Example | compound | figure (MW) | value (MW) | method |
| 7(1) | EX28NG | 4389.9 | 4388.8 | MALDI |
| 7(2) | EX28NL | 4552.0 | 4551.0 | MALDI |
| 7(3) | EX28NS6 | 4843.3 | 4843.9 | MALDI |
| 7(4) | EX28NS3 | 4843.3 | 4840.6 | ESI |
| 7(5) | EX28NS36 | 5134.5 | 5131.9 | ESI |
| 8 | EX-1NG | 4504.0 | 4502.8 | ESI |
| 8 | EX01NG | 4366.8 | 4366.4 | ESI |
| 8 | EX02NG | 4446.9 | 4445.9 | ESI |
| 8 | EX03NG | 4374.8 | 4375.0 | ESI |
| 8 | EX04NG | 4446.9 | 4446.5 | ESI |
| 8 | EX05NG | 4402.9 | 4402.5 | ESI |
| 8 | EX06NG | 4356.8 | 4355.7 | ESI |
| 8 | EX07NG | 4402.9 | 4402.4 | ESI |
| 8 | EX08NG | 4416.9 | 4416.3 | ESI |
| 8 | EX09NG | 4388.9 | 4388.2 | ESI |
| 8 | EX10NG | 4390.8 | 4390.5 | ESI |
| 8 | EX11NG | 4416.9 | 4415.4 | ESI |
| 8 | EX12NG | 4375.8 | 4375.3 | ESI |
| 8 | EX12NL | 4537.9 | 4536.3 | ESI |
| 8 | EX12NS6 | 4829.2 | 4827.3 | ESI |
| 8 | EX13NG | 4375.8 | 4380.4 | ESI |
| 8 | EX13NL | 4538.0 | 4537.0 | ESI |
| 8 | EX13NS6 | 4829.2 | 4827.4 | ESI |
| 8 | EX14NG | 4372.8 | 4377.5 | ESI |
| 8 | EX15NG | 4374.8 | 4373.5 | ESI |
| 8 | EX16NG | 4374.8 | 4379.4 | ESI |
| 8 | EX16NL | 4537.0 | 4535.7 | ESI |
| 8 | EX16NS6 | 4828.2 | 4828.0 | ESI |
| 8 | EX17NG | 4374.8 | 4373.7 | ESI |
| 8 | EX17NL | 4537.0 | 4535.5 | ESI |
| 8 | EX17NS6 | 4828.2 | 4826.3 | ESI |
| 8 | EX18NG | 4432.9 | 4431.9 | ESI |
| 8 | EX19NG | 4404.8 | 4403.7 | ESI |

TABLE 6

| | | theoretical | measured | ionization |
|---|---|---|---|---|
| Example | compound | figure (MW) | value (MW) | method |
| 8 | EX20NG | 4347.8 | 4346.5 | ESI |
| 8 | EX20NL | 4509.9 | 4508.8 | ESI |
| 8 | EX20NS6 | 4801.2 | 4799.2 | ESI |
| 8 | EX21NG | 4390.8 | 4389.6 | ESI |
| 8 | EX21NL | 4552.9 | 4551.8 | ESI |
| 8 | EX21NS6 | 4844.2 | 4841.8 | ESI |
| 8 | EX22NG | 4356.8 | 4355.4 | ESI |
| 8 | EX23NG | 4390.8 | 4389.4 | ESI |
| 8 | EX24NG | 4374.8 | 4373.6 | ESI |
| 8 | EX24NL | 4537.0 | 4535.6 | ESI |
| 8 | EX24NS6 | 4828.2 | 4826.3 | ESI |
| 8 | EX25NG | 4317.7 | 4316.5 | ESI |
| 8 | EX25NL | 4479.9 | 4478.5 | ESI |
| 8 | EX25NS6 | 4771.1 | 4769.2 | ESI |
| 8 | EX26NG | 4390.8 | 4390.3 | ESI |
| 8 | EX27NG | 4375.8 | 4375.6 | ESI |
| 8 | EX27NL | 4537.9 | 4537.2 | ESI |
| 8 | EX27NS6 | 4829.2 | 4827.5 | ESI |
| 8 | EX29NG | 4446.9 | 4446.3 | ESI |
| 8 | EX29NL | 4609.1 | 4607.6 | ESI |
| 8 | EX29NS6 | 4900.3 | 4898.4 | ESI |
| 8 | EX30NG | 4446.9 | 4445.9 | MALDI |
| 8 | EX30NL | 4609.1 | 4608 | MALDI |
| 8 | EX30NS6 | 4900.3 | 4900.6 | MALDI |
| 8 | EX31NG | 4406.8 | 4406.5 | ESI |
| 8 | EX31NL | 4569.0 | 4567.7 | ESI |
| 8 | EX31NS6 | 4860.2 | 4858.7 | ESI |
| 8 | EX32NG | 4416.9 | 4416.5 | ESI |
| 8 | EX32NL | 4579.0 | 4577.8 | ESI |
| 8 | EX32NS6 | 4870.3 | 4868.3 | ESI |
| 8 | EX33NG | 4416.9 | 4416.5 | ESI |
| 8 | EX33NL | 4579.0 | 4577.7 | ESI |
| 8 | EX33NS6 | 4870.3 | 4868.3 | ESI |
| 8 | EX34NG | 4446.9 | 4446.5 | ESI |
| 8 | EX34NL | 4609.1 | 4607.9 | ESI |
| 8 | EX34NS6 | 4900.3 | 4898.3 | ESI |
| 8 | EX35NG | 4432.9 | 4432.6 | ESI |
| 8 | EX35NL | 4595.0 | 4594.3 | ESI |
| 8 | EX35NS6 | 4886.3 | 4884.6 | ESI |

TABLE 7

| | | theoretical | | |
|---|---|---|---|---|
| | | figure | measured | ionization |
| Example | compound | (MW) | value (MW) | method |
| 8 | EX2840NG | 4707.2 | 4706.1 | ESI |
| 8 | EX2840NL | 5031.4 | 5029.7 | ESI |
| 8 | EX2840NS6 | 5614.0 | 5611.6 | ESI |
| 8 | EX17212840NG | 5099.5 | 5096.8 | ESI |
| 8 | EX17213540NG | 5142.5 | 5139.8 | ESI |
| 8 | EX17283540NG | 5141.6 | 5139.0 | ESI |
| 8 | EX21283540NG | 5157.5 | 5155.1 | ESI |
| 8 | EX(1-28) | 3294.7 | 3292.6 | ESI |
| 8 | EX(1-28)28NG | 3497.9 | 3498.8 | ESI |
| 8 | EX(1-28)28NL | 3660.1 | 3660.3 | ESI |
| 8 | EX(1-28)28NS6 | 3951.3 | 3949.3 | ESI |
| 9(1) | EX28C | 4175.7 | 4173.9 | ESI |
| 9(2) | EX28CE1 | 6553.9 | 6550.2 | ESI |
| 9(3) | EX28CJ3Gal | 5146.6 | 5144.2 | ESI |
| 9(4) | EX21283540CJ3Gal | 8184.4 | 8184.8 | ESI |
| 9(5) | EX28C7M | 5367.8 | 5367 | ESI |
| 9(6) | EX21283540C7M | 9069.1 | 9063 | ESI |
| 9(7) | EX28CGlc | 4394.9 | 4393 | ESI |
| 9(7) | EX28CGal | 4394.9 | 4393 | ESI |
| 9(7) | EX28CLac | 4557.0 | 4555 | ESI |
| 9(7) | EX28CGen | 4557.0 | 4555 | ESI |
| 9(7) | EX2840C7M | 6663.0 | 6657.9 | ESI |
| 9(7) | EX21283540C | 4300.9 | 4299.5 | ESI |
| 9(7) | EX21283540CGlc | 5177.7 | 5174.8 | ESI |
| 9(7) | EX21283540CGal | 5177.7 | 5175.1 | ESI |
| 9(7) | EX21283540CLac | 5826.2 | 5822.8 | ESI |
| 9(7) | EX21283540CGen | 5826.2 | 5823.1 | ESI |
| 9(8) | EX28CSLac6 | 4848.3 | 4854.9 | ESI |
| 9(9) | EX21283540CSLac6 | 6991.3 | 6996.9 | ESI |

A method for preparing a reagent used for glycosylation reaction.

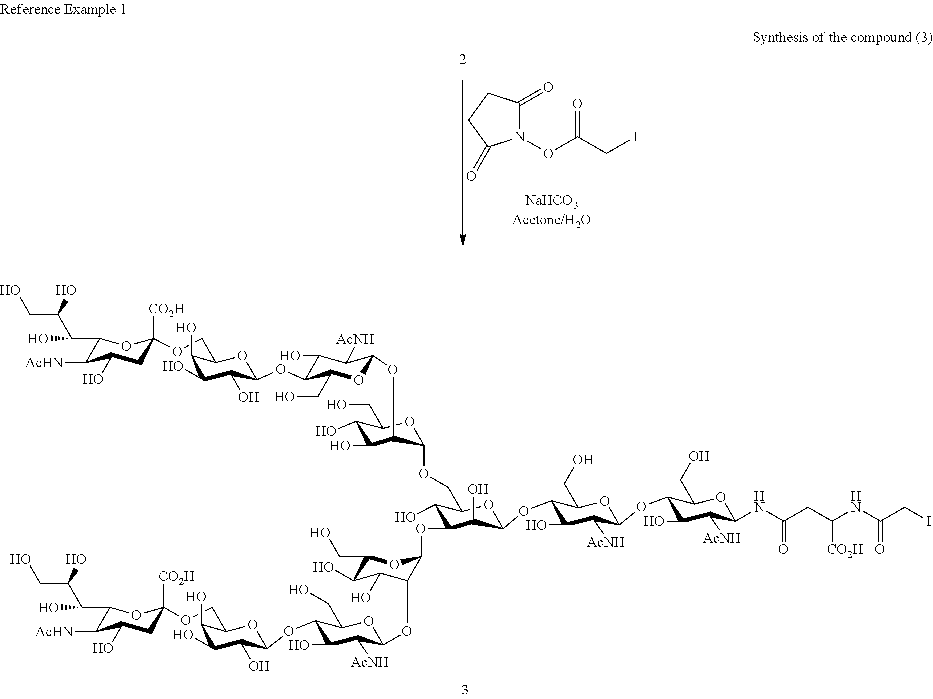

An aqueous solution (5 mL) containing the compound (2)(2 mM), iodoacetic acid N-hydroxysuccinimide ester (10 mM), sodium bicarbonate (15 mM) and 50% (v/v) acetone was reacted at room temperature for 1.5 hours and the reaction was stopped by adding ammonia water (100 mM, 0.5 mL) The reaction solution was neutralized by adding a 1N acetic acid aq. solution and acetone was evaporated in vacuo. The residue was purified with a reversed phase HPLC(Inertsil ODS-3 10×250 mm, GL Science) using 0.1% TFA aq. solution and 0.1% TFA acetonitrile as an eluent, and a gel filtration chromatography (Sephadex G-15) using water as a mobile phase to give the compound (3)(4.1 mg). The product was identified with ESI-MS.

ESI-MS: [M+2H]²⁺=1252.7, (theoretical value: [M+2H]²⁺=1254.0).

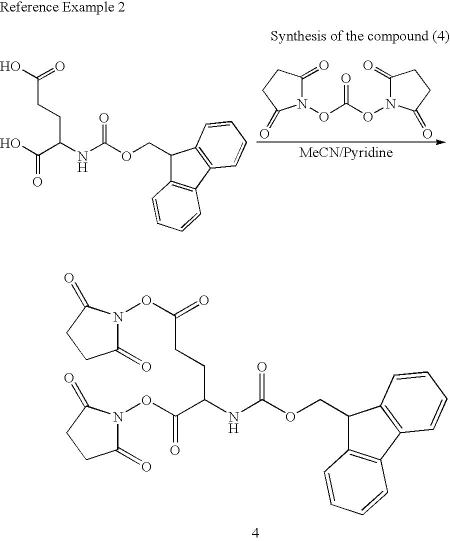

Fmoc-Glu-OH(189 mg), DSC(N,N′-Disuccinimidyl carbonate)(512 mg) and pyridine (158 mg) were dissolved in acetonitrile and heated to reflux for 5 hours. After being cooled to room temperature, acetonitrile was evaporated in vacuo. Ethyl acetate was added to the residue, the organic solution was washed with 1N hydrochloric acid and brine, dried over magnesium sulfate and the solvent was evaporated in vacuo to give a mixture containing the compound (4)(0.36 g). It was used in the next step without further purification.

MALDI-TOF-MS: [M(average)+Na]⁺=587.3, (theoretical value: [M(average)+Na]⁺=586.50),

¹H-NMR (CDCl₃): δ7.77 (d, 2H), 7.61 (br, 2H), 7.41 (t, 2H), 7.32 (t, 7.32), 5.67 (d, 1H), 4.90 (m, 1H), 4.50-4.39 (m, 2H), 4.24 (t, 1H), 2.84 (br, 8H), 2.95-2.65 (m, 2H), 2.47 (m, 1H), 2.36 (m, 1H).

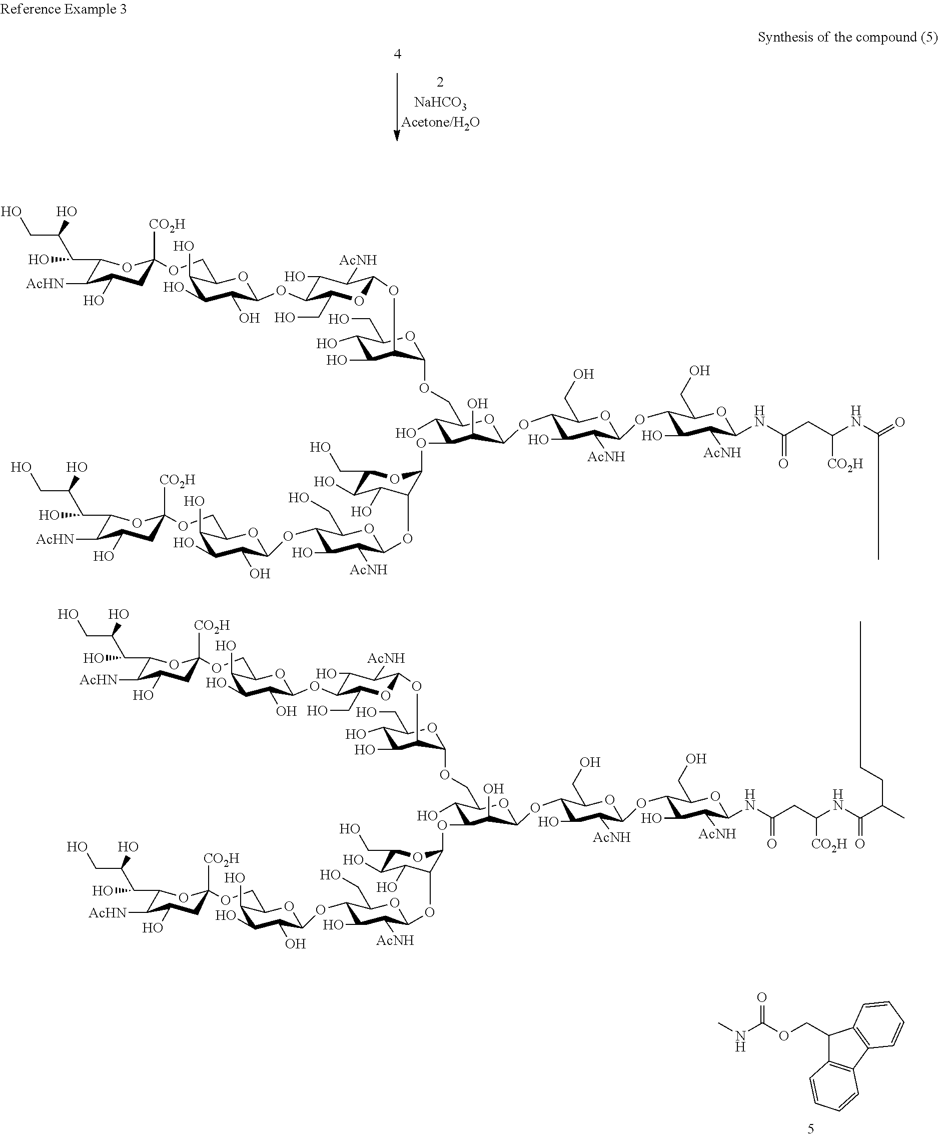

An aqueous solution (1 mL) containing the compound (2)(5 mM), the compound (4)(2 mM), sodium bicarbonate (10 mM) and 50% (v/v) acetone was reacted at room temperature for 3 hours. An aqueous solution of the compound (2)(20 mM, 125 μl) was added and the solution was further reacted for 1.5 hours, and the reaction was stopped by adding ammonia water (100 mM, 50 μl). After the solution was neutralized by adding a 1N acetic acid aq. solution, acetone was evaporated in vacuo and the residue was purified with a reversed phase HPLC(Inertsil ODS-3 10×250 mm, GL Science) using 0.1% TFA aq. solution and 0.1% TFA acetonitrile as an eluent to give the compound (5)(2.2 mg).

MALDI-TOF-MS: [M(average)+Na]⁺=5037.8, (theoretical value: [M(average)+Na]⁺=5032.5).

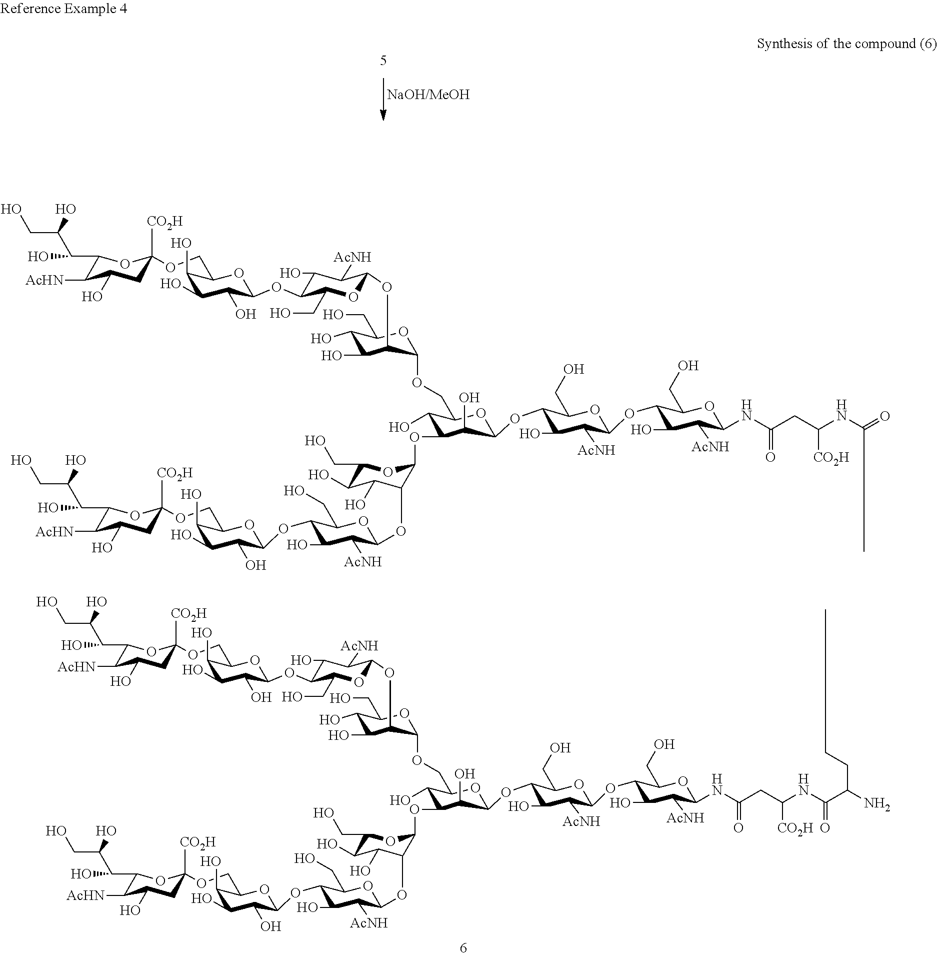

An aqueous solution (1.7 mL) containing the compound (5)(2.2 mg), sodium hydroxide (22 mM) and 88% (v/v) methanol was reacted at room temperature. A 1N sodium hydroxide aq. solution (15μ) was added and the solution was further reacted for 2 hours. The reaction was stopped by neutralization with a 1N acetic acid aq. solution, methanol was evaporated in vacuo and water was added to the residue and the aqueous solution was washed with diethyl ether twice. The washed aqueous layer was purified with a gel filtration chromatography (Sephadex G-15) using water as a mobile phase to give a mixture containing the compound (6)(2.4 mg). It was used in the next step without further purification.

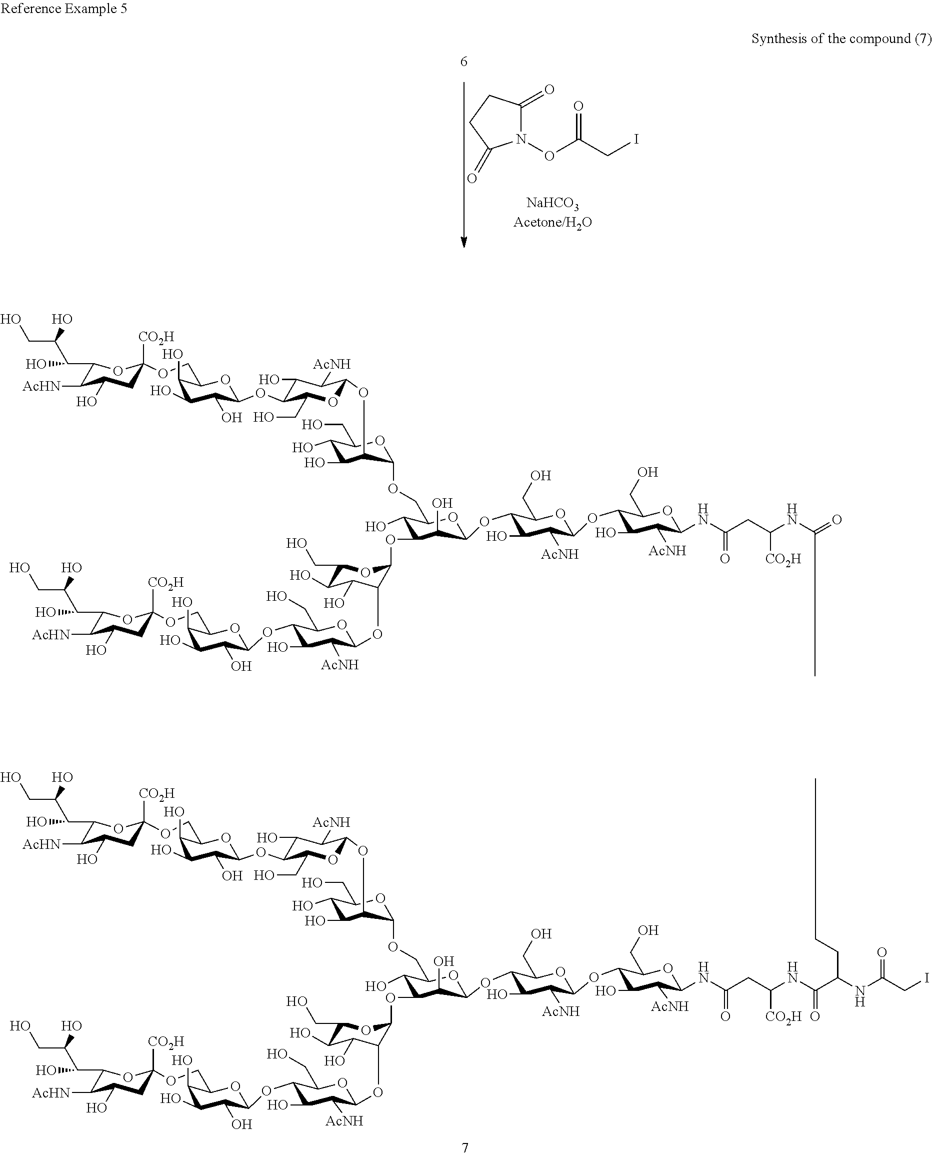

An aqueous solution (0.4 mL) containing the compound (6)(1 mM), iodoacetic acid N-hydroxysuccinimide ester (5 mM), sodium bicarbonate (10 mM) and 50% (v/v) acetone was reacted at room temperature for 2 hours and the reaction was stopped by adding ammonia water (100 mM, 40 μl).

The reaction solution was neutralized by adding a 1N acetic acid aq. solution and acetone was evaporated in vacuo. The residue was purified with a reversed phase HPLC(Inertsil ODS-3 10×250 mm, GL Science) using 0.1% TFA aq. solution and 0.1% TFA acetonitrile as an eluent to give the compound (7)(1.5 mg). The product was identified with MALD-TOF-MS.

MALDI-TOF-MS: [M(average)+Na]⁺=4979.7, (theoretical value: [M(average)+Na]⁺=4978.2).

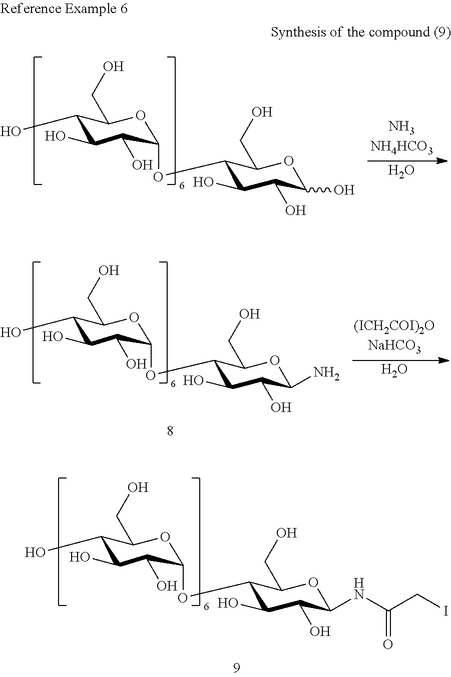

Sodium bicarbonate (1.6 mg) and maltheptaose (23 mg) were dissolved in 16M ammonia water (0.1 mL) and reacted at 42° C. for 36 hours. After the reaction, the solution was concentrated in vacuo, lyophilized to give a mixture containing the compound (8). It was dissolved in a 1M sodium bicarbonate solution (0.2 mL), iodoacetic anhydride (35 mg) was added therein and the mixture was reacted at room temperature for an hour. Then the reaction was stopped by adding 1M ammonia water (0.1 mL). The reaction solution was neutralized by acetic acid and purified with C30 column RPAQUEOUS AR-5 10×250 mm (NOMURA CHEMICAL CO., LDP.) to give the compound (9)(4.5 mg).

ESI-MS: [M+H]⁺=11320.1, (theoretical value: [M+H]⁺=1320.3).

### Reference Example 7

### Synthesis of the Other Iodoacetyl Glycoside

The compound (10), (11), (12) and (13) were synthesized in the same manner as the compound (9).

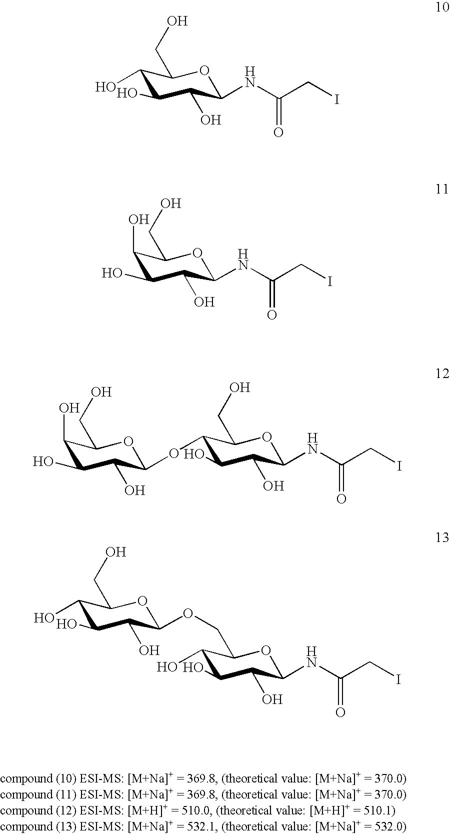

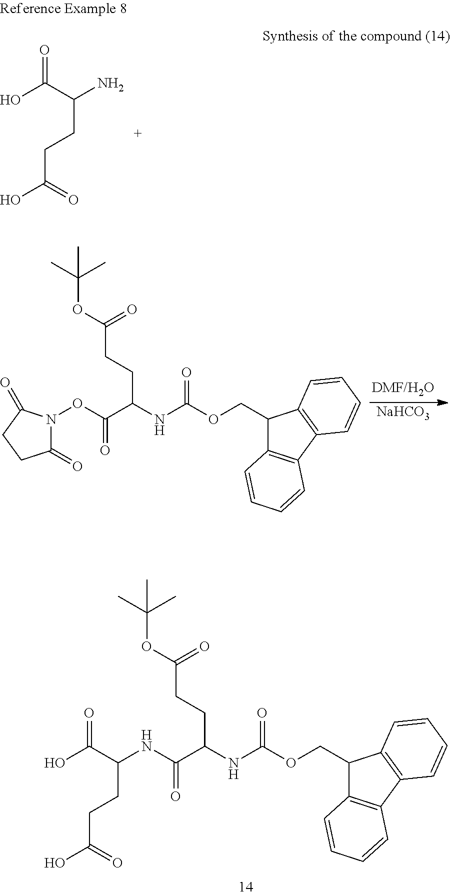

Sodium bicarbonate (3.6 g) and glutamic acid (2.1 g) were added to a mixture of water (50 mL) and DMF(20 mL) and cooled to under 10° C. A solution of Fmoc-Glu (OtBu)-OSu in DMF was poured into the solution and DMF(20 mL) was further added and the mixture was reacted at room temperature for 2 hours. After the reaction, the solution was acidified by adding 1N hydrochloric acid and extracted with ethyl acetate. The organic phase was washed with water, dried over magnesium sulfate and the solvent was evaporated in vacuo to give a mixture containing the compound (14)(1.6 g). It was used in the next step without further purification.

MALDI-TOF-MS: [(average)+Na]⁺=577.35, (theoretical value: [M(average)+Na]⁺=577.58).

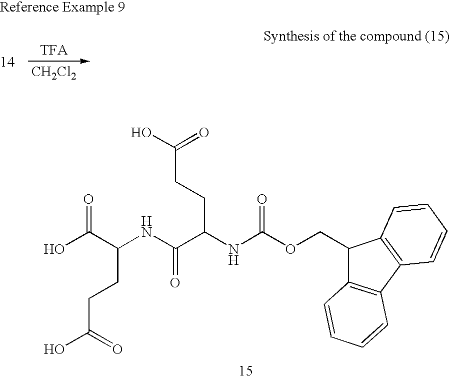

The mixture of the compound (14)(1.5 g) was dissolved in 50% TFA dichloromethane solution and the mixture was stirred at room temperature for an hour. The solvent was evaporated in vacuo, diethyl ether was added to the residue to give the precipitate, which was filtered and dried to give a mixture containing the compound (15)(1.2 g). It was used in the next step without further purification.

MALDI-TOF-MS: [(average)+H]⁺=499.30, (theoretical value:

[M(average)+H]⁺=499.49).

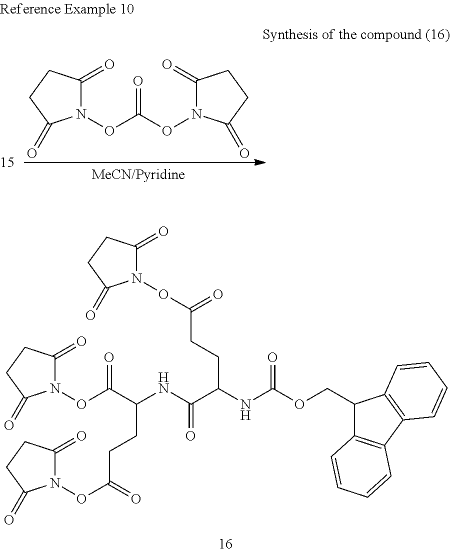

The compound (15)(0.71 g), DSC(N,N′-disuccinimidyl carbonate, 2.2 g) and pyridine (0.68 g) were dissolved in acetonitorile and heated under reflux for 10 hours. Then, the reaction solution was cooled to room temperature and acetonitrile was evaporated in vacuo. Ethyl acetate was added to the residue and the organic layer was washed with 1N hydrochloric acid and brine, dried over magnesium sulfate and the solvent was evaporated to give a mixture containing the compound (16)(0.92 g). It was used in the next step without further purification.

MALDI-TOF-MS: [M(average)+Na]⁺=812.36, (theoretical value: [M(average)+Na]⁺=812.69).

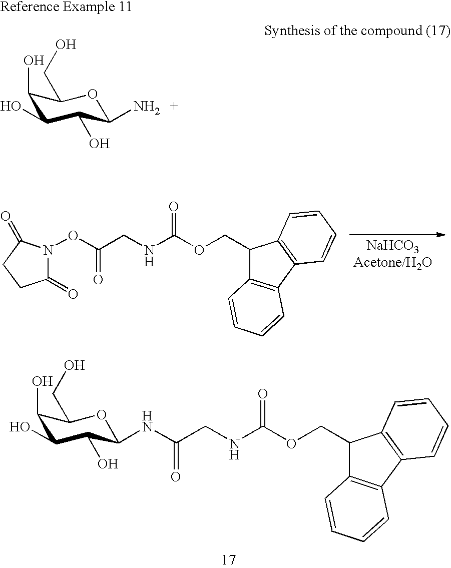

1-Amino-1-deoxy-β-D-galactose (175 mg) and sodium bicarbonate (336 mg) were dissolved in 50% acetone aq. solution (50 mL) and a solution of Fmoc-Gly-OSu (788 mg) in acetone was added therein. Acetone (20 mL) was further added and the mixture was stirred at room temperature for 3.5 hours. Then acetone was evaporated and the resulting aq. solution was purified with a reversed phase HPLC(YMC Pack, ODS-A 20×250 mm) using 0.1% TFA aq. solution and 0.1% TFA acetonitrile as an eluent to give the compound (17)(96 mg).

ESI-MS: [(average)+H]⁺−459.3, (theoretical value: [(average)+H]⁺=459.5).

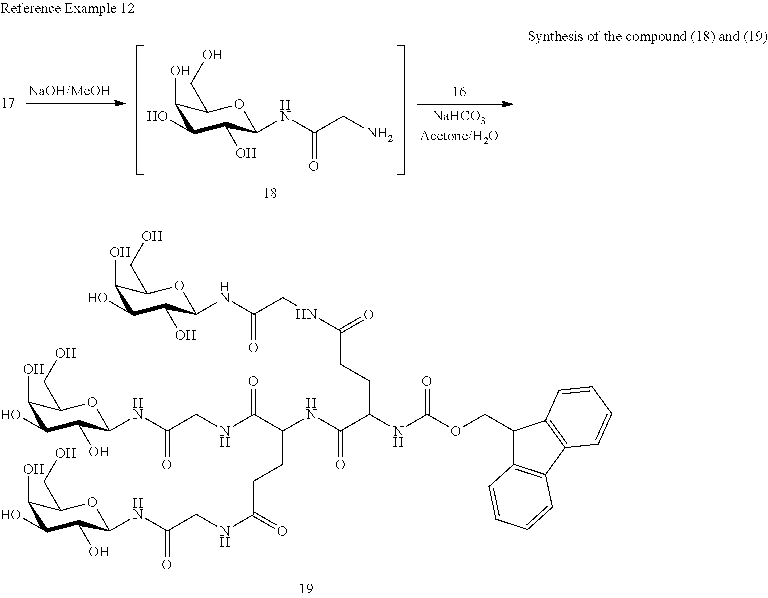

The compound (17)(60 mg) was dissolved in methanol (25 mL), adjusted to pH 12-13 by adding a 1N sodium hydroxide aq. solution and the mixture was reacted at room temperature for 3.5 hours. Then, the reaction solution was neutralized with acetic acid, methanol was evaporated in vacuo, water was added to the residue and the aq. solution was washed with diethyl ether. The aq. solution was concentrated in vacuo and remaining diethyl ether was removed to give an aq. solution (about 2 mL) containing the compound (18). Acetone (1 mL) was added and adjusted to pH 7.0-7.5 by adding a sodium bicarbonate aq. solution (500 mM) and a solution of the compound (16)(21 mg) in acetone was added. After stirring at room temperature for 0.5 hour, a solution of the compound (16)(8 mg) in acetone and the mixture was reacted for additional 1 hour. After the reaction, the reaction solution was neutralized with acetic acid and acetone was evaporated in vacuo. The resulting residue was purified with a reversed phase HPLC(YMC Pack, ODS-A 20×250 mm) using 0.1% TFA aq. solution and 0.1% TFA acetonitrile as an eluent to give the compound (19)(13 mg).

ESI-MS: [M(average)+H]⁺=1153.7, (theoretical value: [M(average)+H]⁺=1154.1).

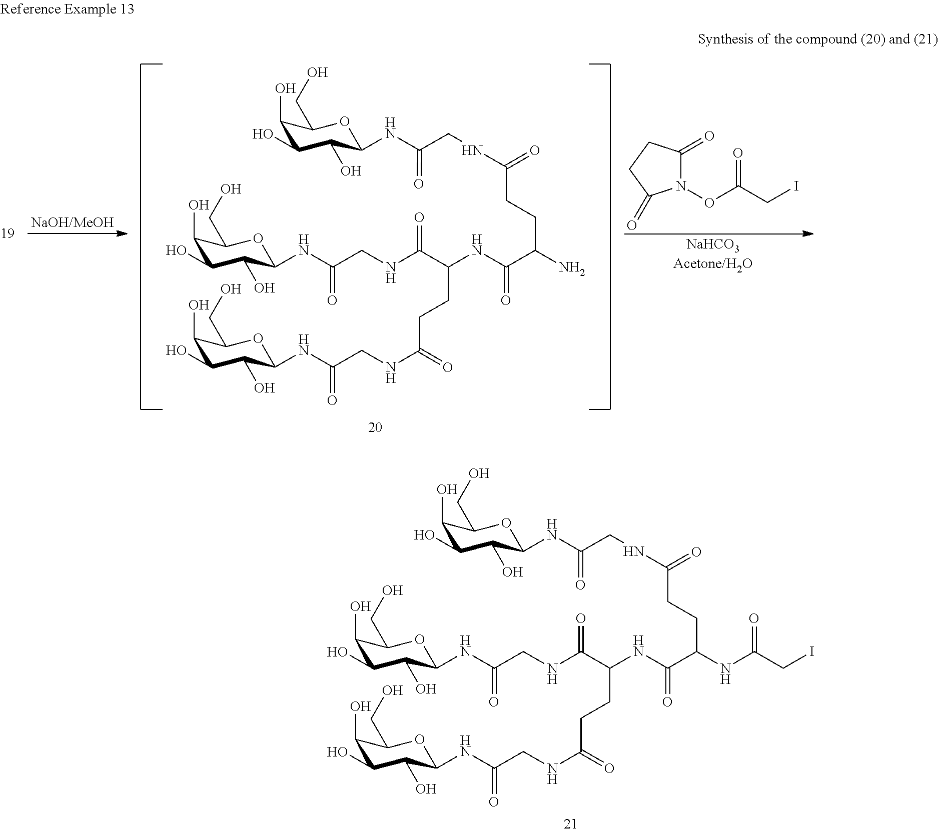

The compound (19)(13 mg) was dissolved in a 50% methanol aq. solution (6 mL) and the mixture was adjusted to pH 12-13 by adding a 1N sodium hydroxide aq. solution and reacted at room temperature for 2 hours. Then, the reaction solution was neutralized with acetic acid, methanol was evaporated in vacuo, water was added to the residue and the aq. solution was washed with diethyl ether. The aq. solution was concentrated in vacuo and remaining diethyl ether was removed to give an aq. solution (about 1.5 mL) containing the compound (20). Acetone (1 mL) was added to the resulting aq. solution, pH of the solution was adjusted to 7.0-7.5 with a sodium bicarbonate aq. solution (500 mM), and a solution of iodoacetic acid N-hydroxysuccimide ester (4.3 mg) in acetone was added and the mixture was reacted at room temperature for 0.5 hour. Then the reaction was stopped by adding ammonium acetate (500 mM, 40 μl) and neutralized with acetic acid. Acetone was evaporated and the residue was purified with a reversed phase HPLC(Inertsil ODS-3 10×250 mm) using 0.1% TFA aq. solution and 0.1% TFA acetonitrileas an eluent to give the compound (21)(3 mg).

ESI-MS: [M(average)+H]⁺=1099.4, (theoretical value: [(average)+H]⁺=1099.8).

1. A glycosylated GLP-1 related peptide having resistance to a degradative enzyme.

2. A glycosylated GLP-1 related peptide comprising GLP-1 (7-36) amide shown in the formula (I) or excendin-4 shown in the formula (II) below;

| | |
|---|---|
| | 7 36 |
| (I): | HAEGTFTSDVSSYLEGQAAKEFIAWLVKGR-NH2 |
| (II): | HGEGTFTSDLSKQMEEEAVRLFIEWLKNGGPSSGAPPPS-NH2 |
| | 1 39 |

wherein one to eleven amino acid(s) are deleted, substituted or added in the said sequences, and further
1) one or more of an amino acid selected from the group of His⁷, Ala⁸, Glu⁹, Thr¹¹, Ser¹⁴, Val¹⁶, Ser¹⁷, Ser¹⁸, Tyr¹⁹, Leu²⁰, Glu²¹, Gly²², Gln²³, Ala²⁴, Ala²⁵, Lys²⁶, GlU²⁷, Ala³⁰, Trp³¹, Leu³², Val³³, Lys³⁴, Gly³⁵ and Arg³⁶ is (are) substituted with an amino acid residue selected from a group of Ser, Thr, Asp, Asn, Glu, Gln and Cys, all of which are glycosilated at their side chains in the sequence of the peptide (I), or
one or more of an amino acid selected from the group of His¹, Gly², Glu³, Thr⁵, Ser⁸, Leu¹⁰, Ser¹¹, Lys¹², Gln¹⁷, Met¹⁴, Glu¹⁵, Glu¹⁶, Glu¹⁷, Ala¹⁸, Val¹⁹, Arg²⁰, Leu²¹, Glu²⁴, Trp²⁵, Leu²⁶, Lys²⁷, Asn²⁸, Gly²⁹, Gly³⁰, Pr³¹, Ser³², Ser³³, Gly³⁴, Ala³⁵, Pro³⁶, Pro³⁷, Pro³⁸ and Ser³⁹ is (are) substituted with an amino acid residue selected from a group of Ser, Thr, Asp, Asn, Glu, Gln and Cys, all of which are glycosilated at their side chains in the sequence of the peptide (II), and/or
2) one or more of an amino acid selected from the added amino acids is (are) substituted with an amino acid residue selected from a group of Ser, Thr, Asp, Asn, Glu, Gln and Cys, all of which are glycosilated at their side chains.

3. A glycosylated GLP-1 related peptide of the peptide derivative of the formula (III) below;
7 8 9 10 11 12 13 14 15 16 17
Xaa Xbb Xcc Gly Xdd Phe Thr Xee Asp Xff Xgg
18 19 20 21 22 23 24 25 26 27 28
Xhh Xii Xjj Xkk Xll Xmm Xnn Xoo Xpp Xqq Phe
29 30 31 32 33 34 35 36 37 38 39
Ile Xrr Xss Xtt Xuu Xvv Xww Xxx Xyy Xzz Yaa
40 41 42 43 44 45
Ybb Ycc Ydd Yee Yff Ygg
wherein Xaa is His or 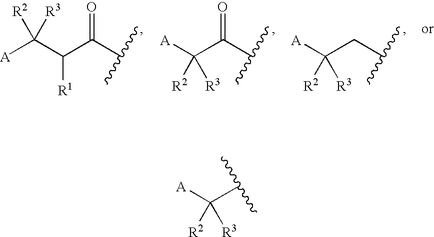
wherein R¹, R² and R³ are independently a hydrogen atom, lower alkyl optionally substituted with aryl, lower alkylcarbonylamino, hydroxyl, lower alkyloxy, a halogen atom, a lower alkylsulfonyl or trifluoromethyl, or R¹ and R² may form a single bond;
wherein aryl may be substituted with a substituent selected from amino, hydroxyl, lower alkyl, lower alkyloxy, a halogen atom, lower alkylsulfonyl, lower alkylcarbonylamino and trifluoromethyl;
A is a cyclic group of 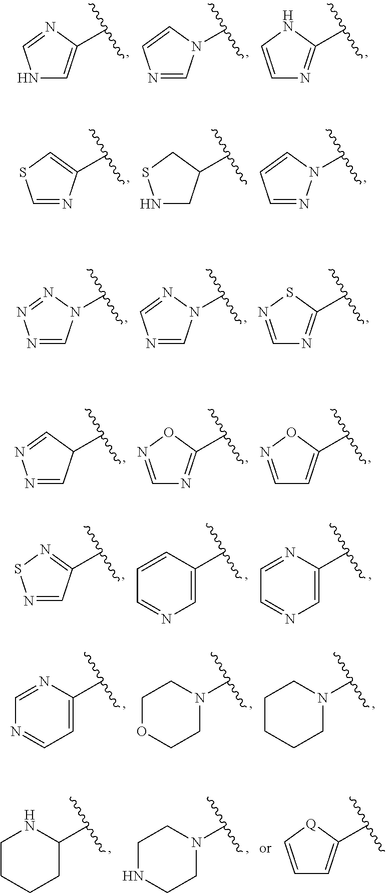
wherein Q is a nitrogen atom, an oxygen atom or a sulfur atom;
and the said cyclic group may be substituted with one or more of substituents selected from amino, nitro, hydroxyl, lower alkyl, lower alkyloxy, a halogen atom, trifluoromethyl and aryl;
Xbb is Ala, D-Ala, Gly, Asp, Glu, Phe, Ile, Leu, Met, Asn, Gln, Ser, Thr, Val, Tyr or a glycosylated amino acid residue selected from a group A of glycosylated amino acid residue containing 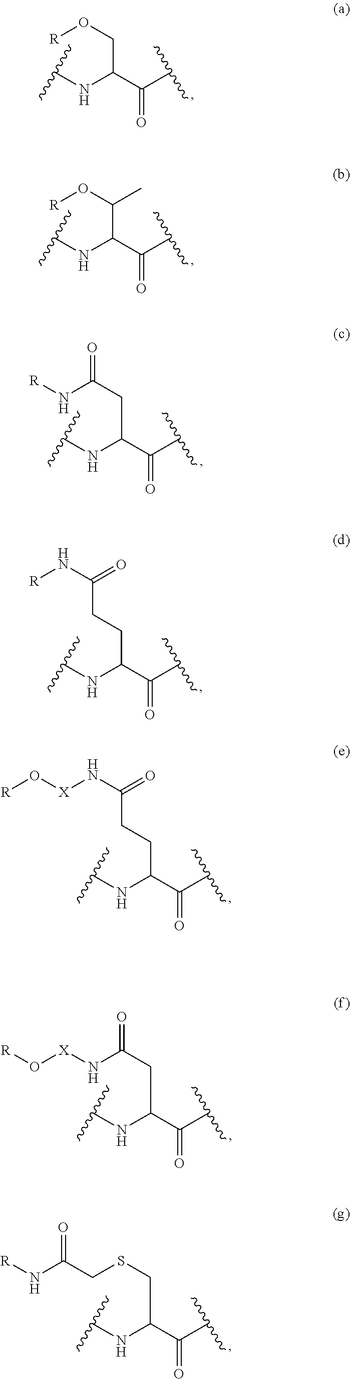 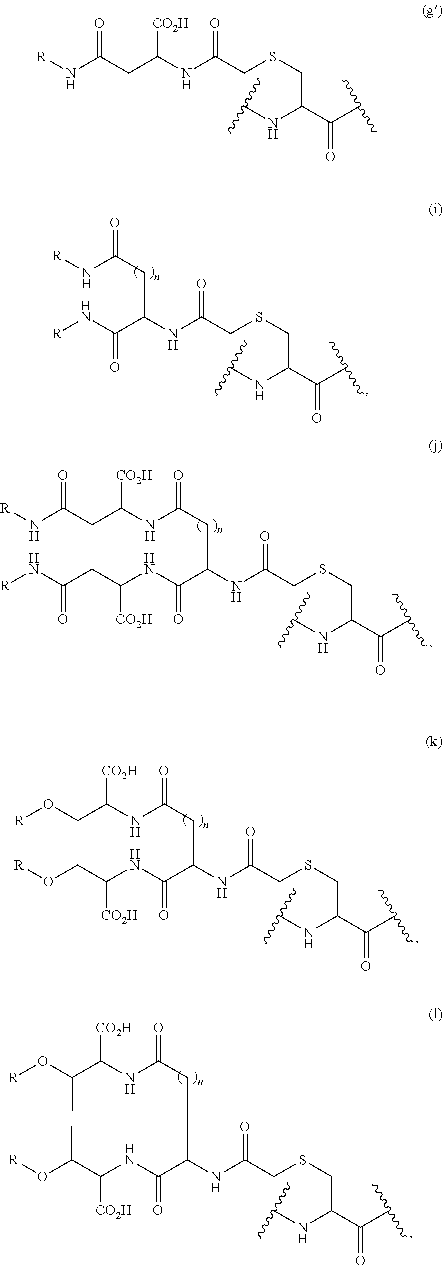 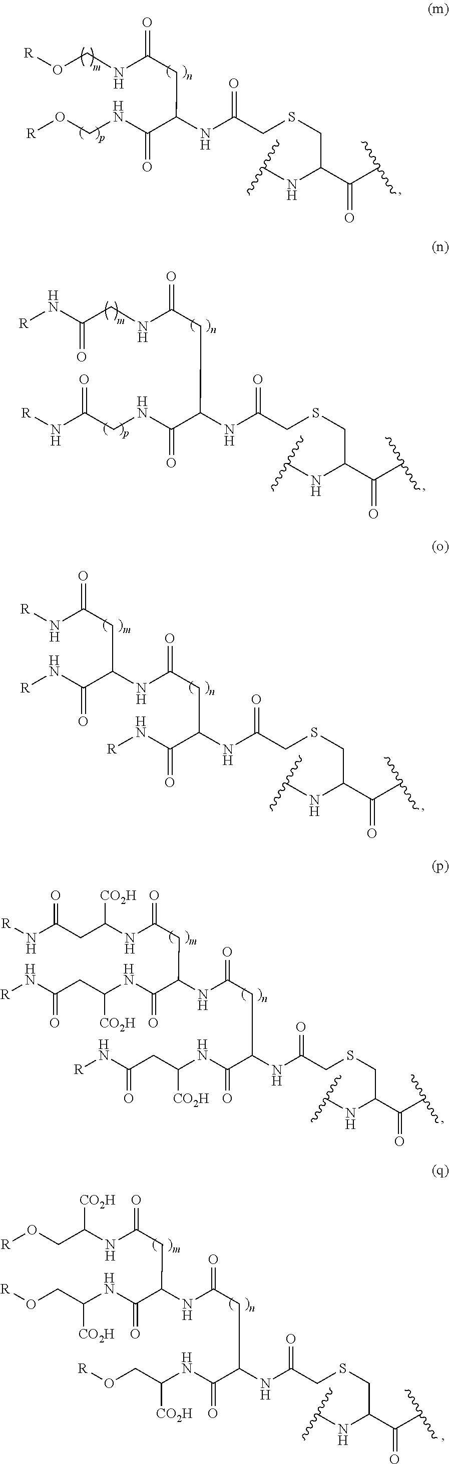 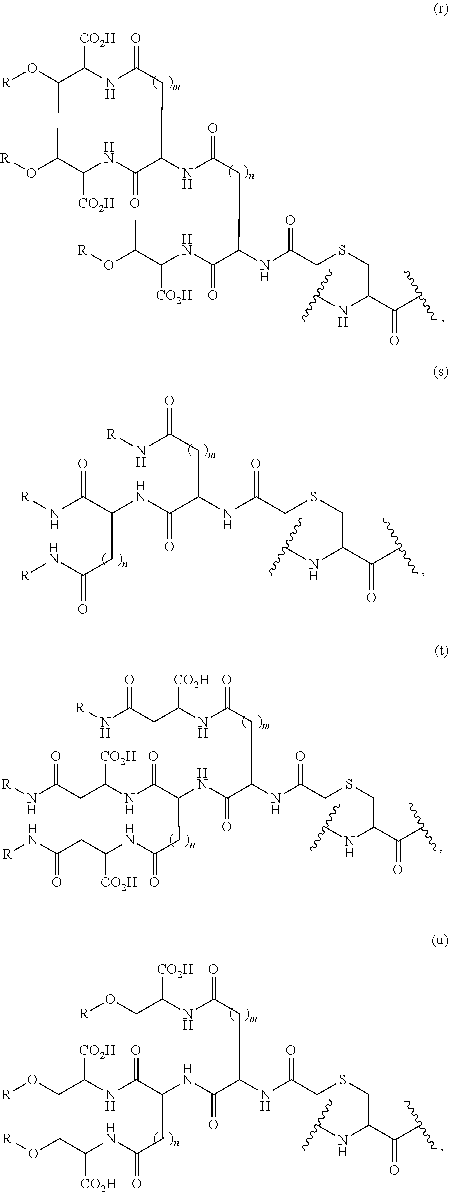 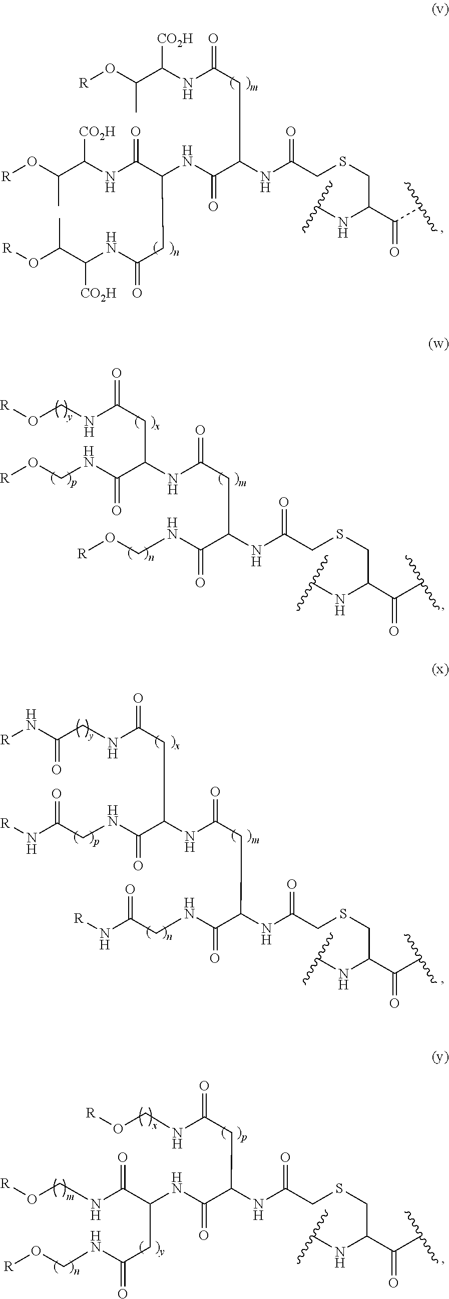 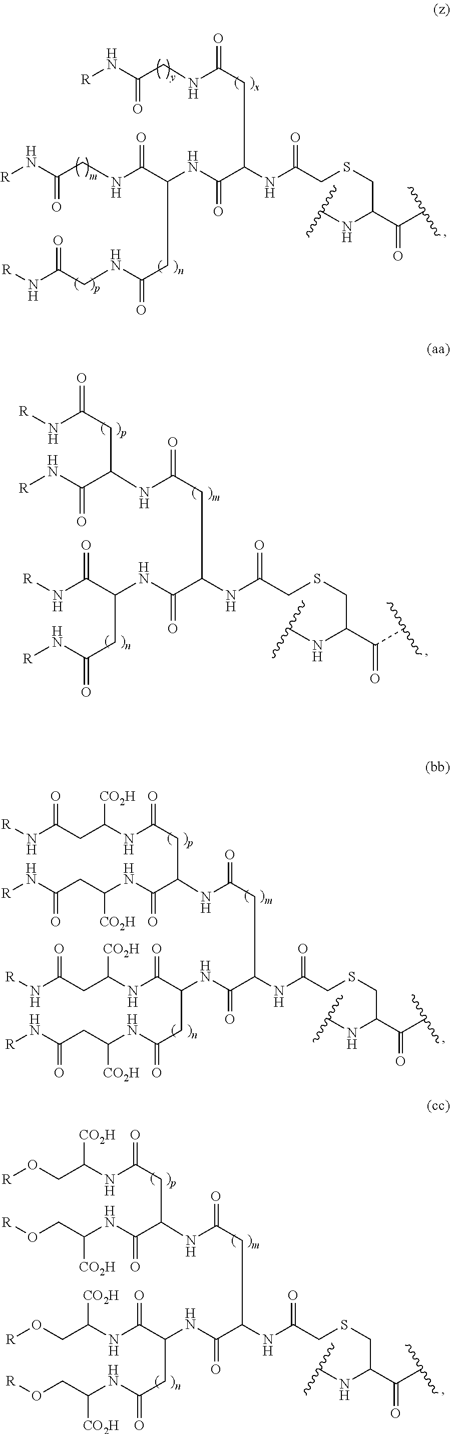 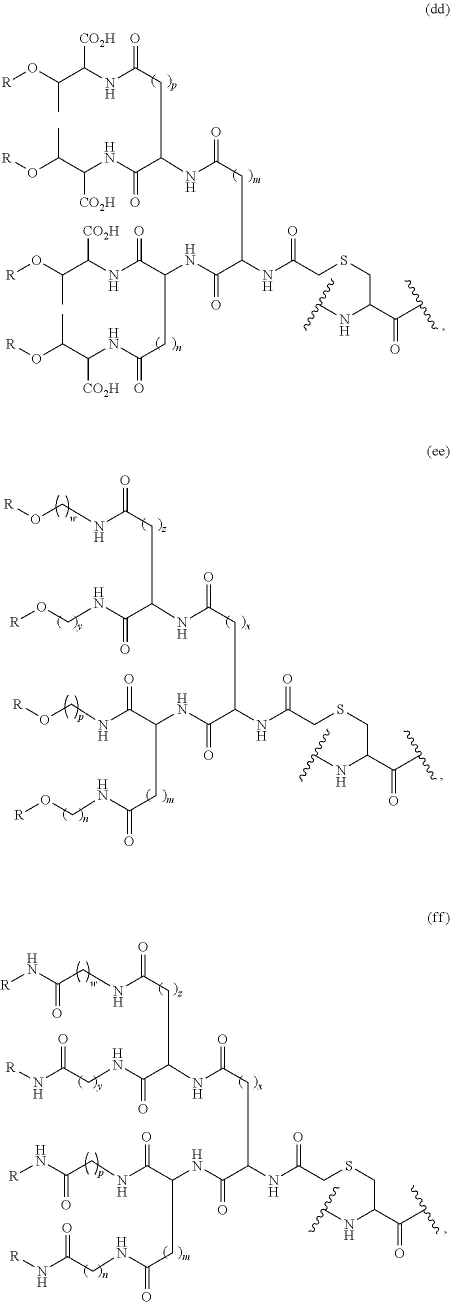 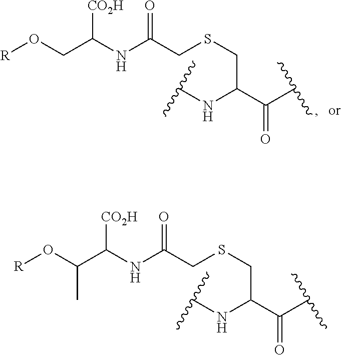
wherein R is independently a glycochain; X, Y and Z are linkers and m, n, p, w, x, y and z are integers of 1 to 10 respectively;
Xcc is Glu or a glycosilated amino acid residue selected from the group A of glycosylated amino acid residues,
Xdd is Thr, Ala, Gly, Ile, Leu, Ser, Val, Asp or a glycosilated amino acid residue selected from the group A of glycosylated amino acid residues,
Xee is Ser, Ala, Gly, Ile, Leu, Thr, Val or a glycosilated amino acid residue selected from the group A of glycosylated amino acid residues,
Xff is Val, Leu, Ala, Gly, Ile, Ser, Thr, Tyr or a glycosilated amino acid residue selected from the group A of glycosylated amino acid residues,
Xgg is Ser, Ala, Gly, Ile, Leu, Thr, Val or a glycosilated amino acid residue selected from the group A of glycosylated amino acid residues,
Xhh is Ser, Lys, Ala, Gly, Ile, Leu, Thr, Val or a glycosilated amino acid residue selected from the group A of glycosylated amino acid residues,
Xii is Tyr, Gln, Phe, Trp, or a glycosilated amino acid residue selected from the group A of glycosylated amino acid residues,
Xjj is Leu, Met, Ala, Gly, Ile, Leu, Ser, Thr, Val or a glycosilated amino acid residue selected from the group A of glycosylated amino acid residues,
Xkk is Glu, Ala, Gly, Gln, Ser, Thr, Asp or a glycosilated amino acid residue selected from the group A of glycosylated amino acid residues,
Xll is Gly, Glu, Ala, Ile, Leu, Ser, Thr, Val, Asp or a glycosilated amino acid residue selected from the group A of glycosylated amino acid residues,
Xmm is Gln, Glu, Asn, Arg, Asp or a glycosilated amino acid residue selected from the group A of glycosylated amino acid residues,
Xnn is Ala, Gly, Ile, Leu, Arg, Ser, Thr, Val or a glycosilated amino acid residue selected from the group A of glycosylated amino acid residues,
Xoo is Ala, Val, Gly, Ile, Leu, Ser, Thr or a glycosilated amino acid residue selected from the group A of glycosylated amino acid residues,
Xpp is Lys, Arg, His, Gln or a glycosilated amino acid residue selected from the group A of glycosylated amino acid residues,
Xqq is Glu, Leu, Ala, Gly, Gln, Ser, Thr, Asp or a glycosilated amino acid residue selected from the group A of glycosylated amino acid residues,
Xrr is Ala, Glu, Gly, Ile, Leu, Ser, Thr, Val, Asp or a glycosilated amino acid residue selected from the group A of glycosylated amino acid residues,
Xss is Trp, Phe, Tyr or a glycosilated amino acid residue selected from the group A of glycosylated amino acid residues,
Xtt is Leu, Ala, Gly, Ile, Ser, Thr, Val or a glycosilated amino acid residue selected from the group A of glycosylated amino acid residues,
Xuu is Val, Lys, Ala, Gly, Ile, Leu, Met, Ser, Thr, Arg or a glycosilated amino acid residue selected from the group A of glycosylated amino acid residues,
Xvv is Lys, Asn, His, Gln, Arg or a glycosilated amino acid residue selected from the group A of glycosylated amino acid residues,
Xww is Gly, Ala, Ile, Leu, Ser, Thr, Val or a glycosilated amino acid residue selected from the group A of glycosylated amino acid residues,
Xxx is Arg, Gly, His, Lys or a glycosilated amino acid residue selected from the group A of glycosylated amino acid residues,
Xyy is Gly, Pro, Ala, Ile, Leu, Ser, Thr, Val or a glycosilated amino acid residue selected from the group A of glycosylated amino acid residues,
Xzz is Arg, Ala, Asp, Glu, Phe, Gly, His, Ile, Lys, Leu, Met, Asn, Pro, Gln, Ser, Thr, Val, Trp, Tyr or a glycosylated amino acid residue selected from the group A of glycosylated amino acid residues, or does not exist,
Yaa is Arg, Ala, Asp, Glu, Phe, Gly, His, Ile, Lys, Leu, Met, Asn, Pro, Gln, Ser, Thr, Val, Trp, Tyr or a glycosylated amino acid residue selected from the group A of glycosylated amino acid residues, or does not exist,
Ybb is Gly, Ala, Asp, Glu, Phe, His, Ile, Lys, Leu, Met, Asn, Pro, Gln, Arg, Ser, Thr, Val, Trp, Tyr or a glycosylated amino acid residue selected from the group A of glycosylated amino acid residues, or does not exist,
Ycc is Ala, Asp, Glu, Phe, Gly, His, Ile, Lys, Leu, Met, Asn, Pro, Gln, Arg, Ser, Thr, Val, Trp, Tyr or a glycosylated amino acid residue selected from the group A of glycosylated amino acid residues, or does not exist,
Ydd is Pro, Ala, Asp, Glu, Phe, Gly, His, Ile, Lys, Leu, Met, Asn, Gln, Arg, Ser, Thr, Val, Trp, Tyr or a glycosylated amino acid residue selected from the group A of glycosylated amino acid residues, or does not exist,
Yee is Pro, Ala, Asp, Glu, Phe, Gly, His, Ile, Lys, Leu, Met, Asn, Gln, Arg, Ser, Thr, Val, Trp, Tyr or a glycosylated amino acid residue selected from the group A of glycosylated amino acid residues, or does not exist,
Yff is Pro, Ala, Asp, Glu, Phe, Gly, His, Ile, Lys, Leu, Met, Asn, Gln, Arg, Ser, Thr, Val, Trp, Tyr or a glycosylated amino acid residue selected from the group A of glycosylated amino acid residues, or does not exist,
Ygg is Ser, Ala, Asp, Glu, Phe, Gly, His, Ile, Lys, Leu, Met, Asn, Pro, Gln, Arg, Thr, Val, Trp, Tyr or a glycosylated amino acid residue selected from the group A of glycosylated amino acid residues, or does not exist, provided that one to six residue(s) of Xaa to Ygg is (are) substituted with a glycosylated amino acid residue selected from the group A of glycosylated amino acid residues and that the number of the residues which does not exist or is different from the corresponding residue when the sequence of Xaa to Ygg and the aminoacid sequence of the peptide (I) or (II) does not exceed 11,
or its ester, amide, alkylamide or dialkylamide; and/or a pharmaceutically acceptable salt thereof.

4. The glycosylated GLP-1 related peptide of claim 3, wherein Xaa is His, Xbb is Ala, Xcc is Glu, Xdd is Thr, Xee is Ser, Xff is Val, Xgg is Ser, Xhh is Ser, Xii is Tyr, Xjj is Leu or a glycosylated amino acid residue selected from the group A of glycosylated amino acid residues,
Xkk is Glu or a glycosylated amino acid residue selected from the group A of glycosylated amino acid residues,
Xll is Gly or a glycosylated amino acid residue selected from the group A of glycosylated amino acid residues,
Xmm is Gln or a glycosylated amino acid residue selected from the group A of glycosylated amino acid residues,
Xnn is Ala or a glycosylated amino acid residue selected from the group A of glycosylated amino acid residues,
Xoo is Ala or a glycosylated amino acid residue selected from the group A of glycosylated amino acid residues,
Xpp is Lys or a glycosylated amino acid residue selected from the group A of glycosylated amino acid residues,
Xqq is Glu or a glycosylated amino acid residue selected from the group A of glycosylated amino acid residues,
Xrr is Ala or a glycosylated amino acid residue selected from the group A of glycosylated amino acid residues,
Xss is Trp or a glycosylated amino acid residue selected from the group A of glycosylated amino acid residues,
Xtt is Leu or a glycosylated amino acid residue selected from the group A of glycosylated amino acid residues,
Xuu is Val or a glycosylated amino acid residue selected from the group A of glycosylated amino acid residues,
Xvv is Lys or a glycosylated amino acid residue selected from the group A of glycosylated amino acid residues,
Xww is Gly or a glycosylated amino acid residue selected from the group A of glycosylated amino acid residues,
Xxx is Arg or a glycosylated amino acid residue selected from the group A of glycosylated amino acid residues, and
Xyy is Gly or a glycosylated amino acid residue selected from the group A of glycosylated amino acid residues.

5. The glycosylated GLP-1 related peptide of claim 3, wherein Xaa is His, Xbb is Ala, Xcc is Glu, Xdd is Thr, Xee is Ser, Xff is Val, Xgg is Ser, Xhh is Ser, Xii is Tyr,
Xjj is Leu or a glycosylated amino acid residue selected from the group A of glycosylated amino acid residues,
Xkk is Glu or a glycosylated amino acid residue selected from the group A of glycosylated amino acid residues,
Xll is Gly or a glycosylated amino acid residue selected from the group A of glycosylated amino acid residues,
Xmm is Gln or a glycosylated amino acid residue selected from the group A of glycosylated amino acid residues,
Xnn is Ala or a glycosylated amino acid residue selected from the group A of glycosylated amino acid residues,
Xoo is Ala or a glycosylated amino acid residue selected from the group A of glycosylated amino acid residues,
Xpp is Lys or a glycosylated amino acid residue selected from the group A of glycosylated amino acid residues,
Xqq is Glu or a glycosylated amino acid residue selected from the group A of glycosylated amino acid residues,
Xrr is Ala or a glycosylated amino acid residue selected from the group A of glycosylated amino acid residues,
Xss is Trp or a glycosylated amino acid residue selected from the group A of glycosylated amino acid residues,
Xtt is Leu or a glycosylated amino acid residue selected from the group A of glycosylated amino acid residues,
Xuu is Val or a glycosylated amino acid residue selected from the group A of glycosylated amino acid residues,
Xvv is Lys or a glycosylated amino acid residue selected from the group A of glycosylated amino acid residues,
Xww is Gly or a glycosylated amino acid residue selected from the group A of glycosylated amino acid residues,
Xxx is Arg or a glycosylated amino acid residue selected from the group A of glycosylated amino acid residues,
Xyy is Gly or a glycosylated amino acid residue selected from the group A of glycosylated amino acid residues, and
the sequence of Xzz to Ygg does not exist.

6. The glycosylated GLP-1 related peptide of claim 4, wherein only Xpp, Xvv and/or Xyy is (are) substituted with a glycosylated amino acid residue selected from the group A of glycosylated amino acid residues.

7. The glycosylated GLP-1 related peptide of claim 5, wherein only Xpp, Xvv and/or Xyy is (are) substituted with a glycosylated amino acid residue selected from the group A of glycosylated amino acid residues.

8. The glycosylated GLP-1 related peptide of claim 3, wherein Xaa is His is Gly, Xcc is Glu, Xdd is Thr, Xee is Ser, Xff is Leu, Xgg is Ser, Xhh is Lys, Xii is Gln,
Xjj is Met or a glycosylated amino acid residue selected from the group A of glycosylated amino acid residues,
Xkk is Glu or a glycosylated amino acid residue selected from the group A of glycosylated amino acid residues,
Xll is Glu or a glycosylated amino acid residue selected from the group A of glycosylated amino acid residues,
Xmm is Glu or a glycosylated amino acid residue selected from the group A of glycosylated amino acid residues,
Xnn is Ala or a glycosylated amino acid residue selected from the group A of glycosylated amino acid residues,
Xoo is Val or a glycosylated amino acid residue selected from the group A of glycosylated amino acid residues,
Xpp is Arg or a glycosylated amino acid residue selected from the group A of glycosylated amino acid residues,
Xqq is Leu or a glycosylated amino acid residue selected from the group A of glycosylated amino acid residues,
Xrr is Glu or a glycosylated amino acid residue selected from the group A of glycosylated amino acid residues,
Xss is Trp or a glycosylated amino acid residue selected from the group A of glycosylated amino acid residues,
Xtt is Leu or a glycosylated amino acid residue selected from the group A of glycosylated amino acid residues,
Xuu is Lys or a glycosylated amino acid residue selected from the group A of glycosylated amino acid residues,
Xvv is Asn or a glycosylated amino acid residue selected from the group A of glycosylated amino acid residues,
Xww is Gly or a glycosylated amino acid residue selected from the group A of glycosylated amino acid residues,
Xxx is Gly or a glycosylated amino acid residue selected from the group A of glycosylated amino acid residues,
Xyy is Pro or a glycosylated amino acid residue selected from the group A of glycosylated amino acid residues,
Xzz is Ser or a glycosylated amino acid residue selected from the group A of glycosylated amino acid residues,
Yaa is Ser or a glycosylated amino acid residue selected from the group A of glycosylated amino acid residues,
Ybb is Gly or a glycosylated amino acid residue selected from the group A of glycosylated amino acid residues,
Ycc is Ala or a glycosylated amino acid residue selected from the group A of glycosylated amino acid residues,
Ydd is Pro or a glycosylated amino acid residue selected from the group A of glycosylated amino acid residues,
Yee is Pro or a glycosylated amino acid residue selected from the group A of glycosylated amino acid residues,
Yff is Pro or a glycosylated amino acid residue selected from the group A of glycosylated amino acid residues, and
Ygg is Ser or a glycosylated amino acid residue selected from the group A of glycosylated amino acid residues.

9. The glycosylated GLP-1 related peptide of claim 3, wherein the glycochain is selected from

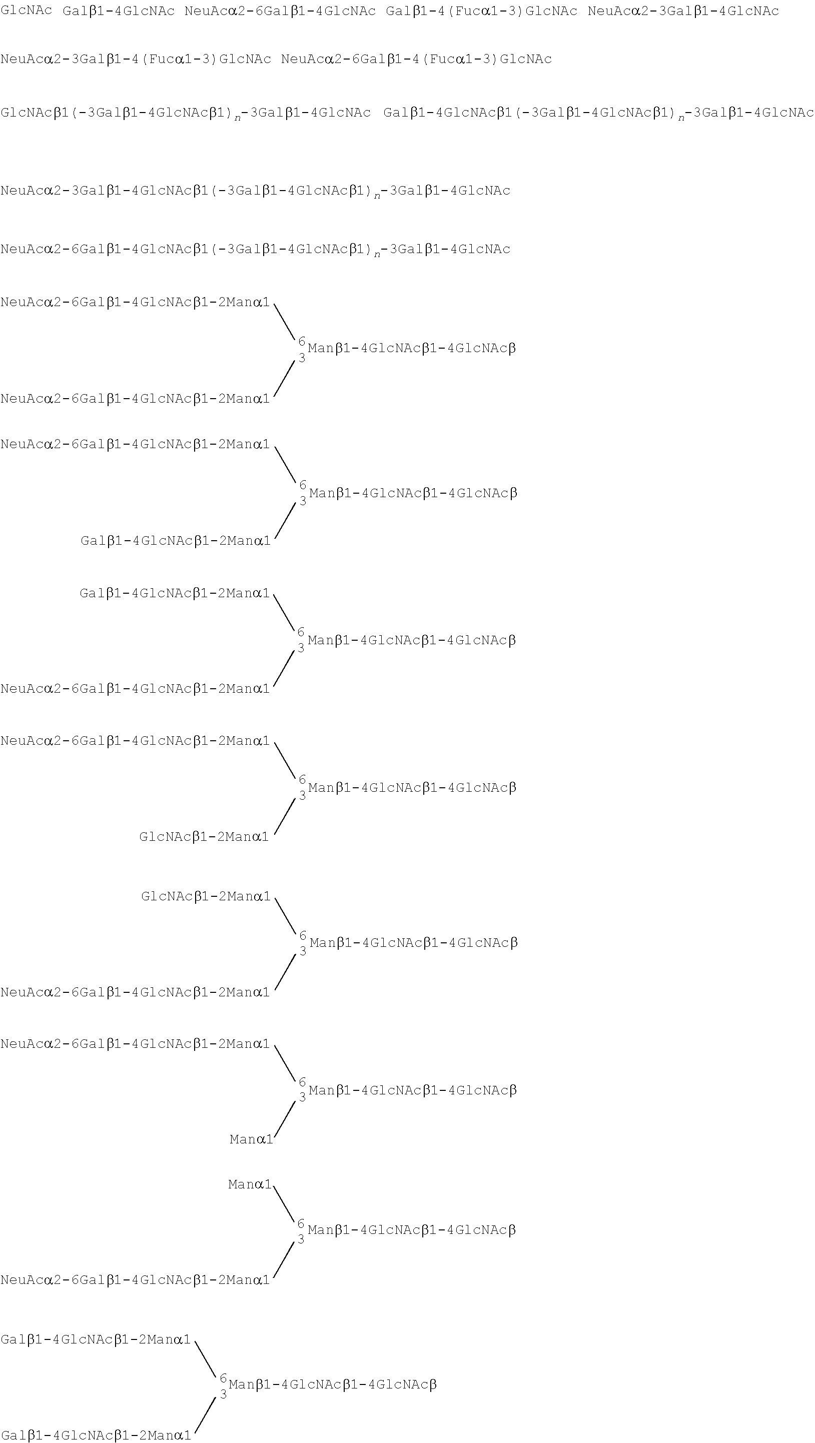

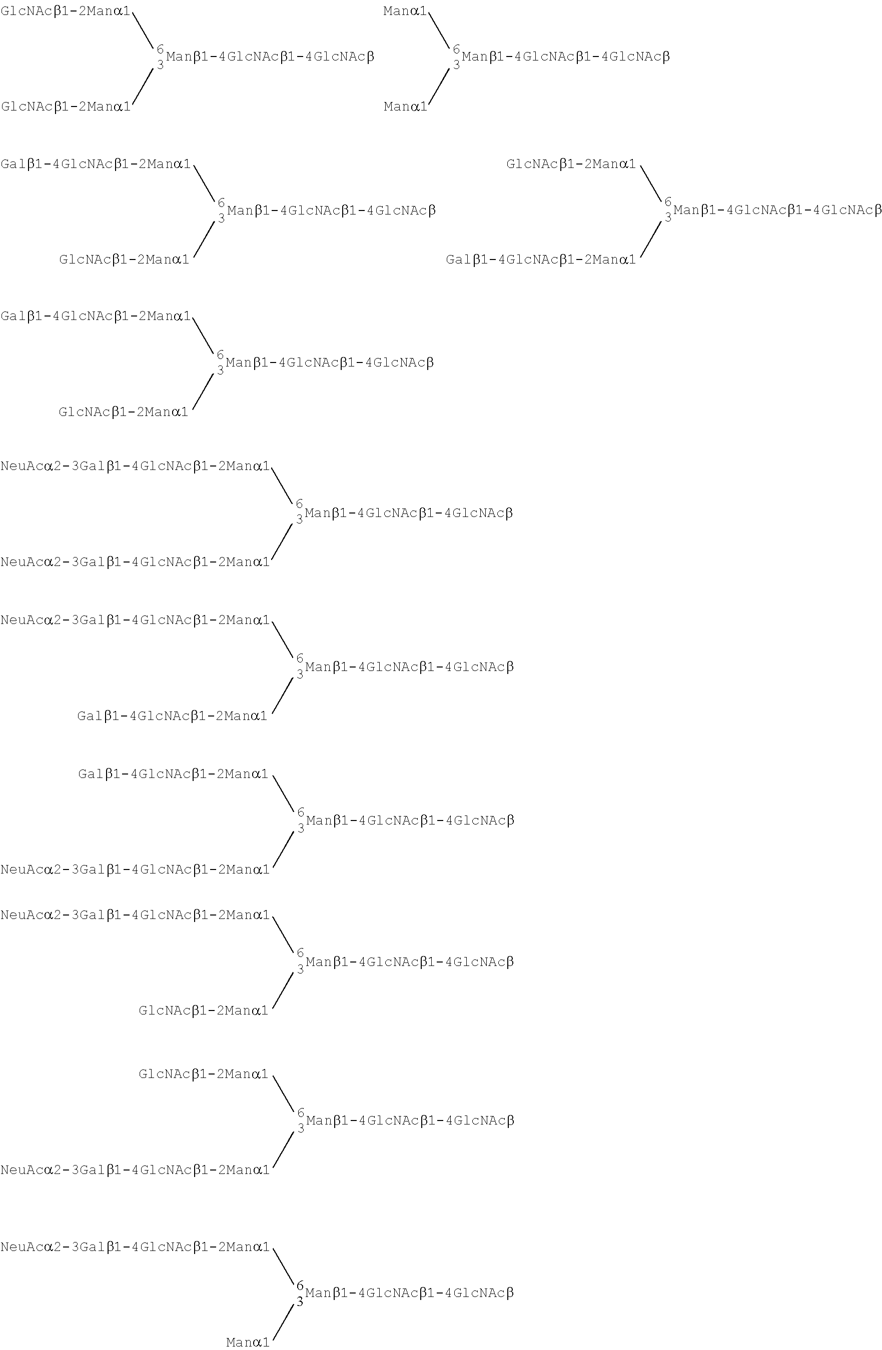

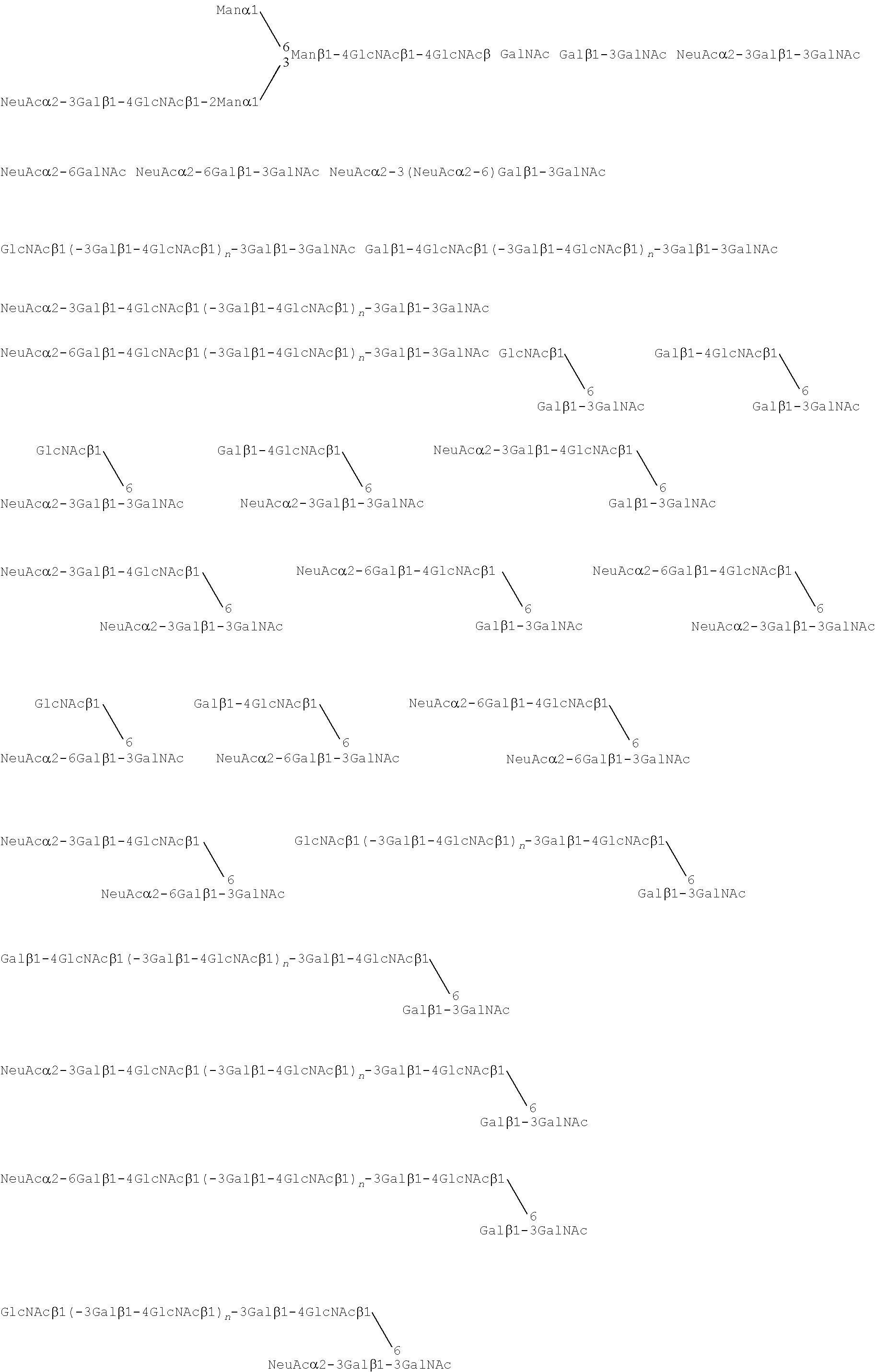

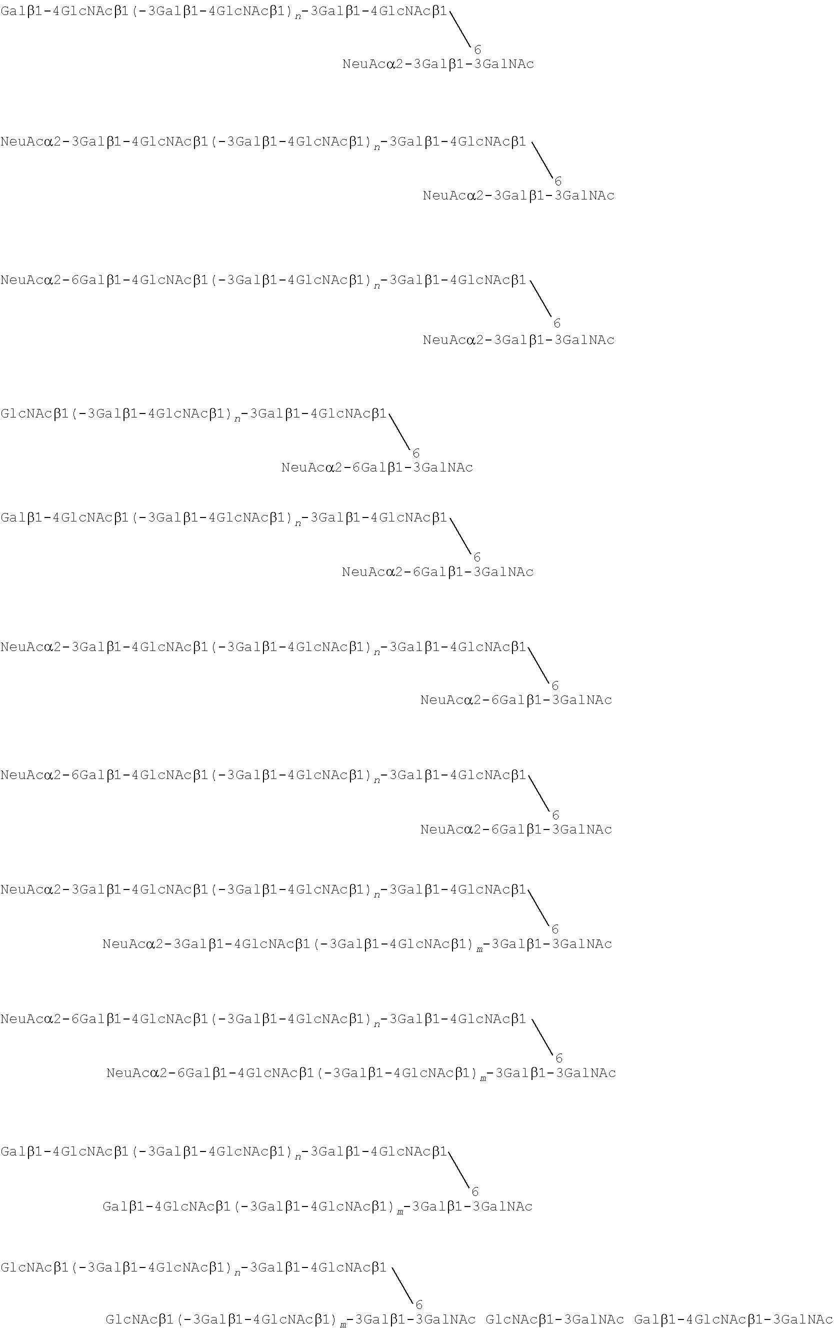

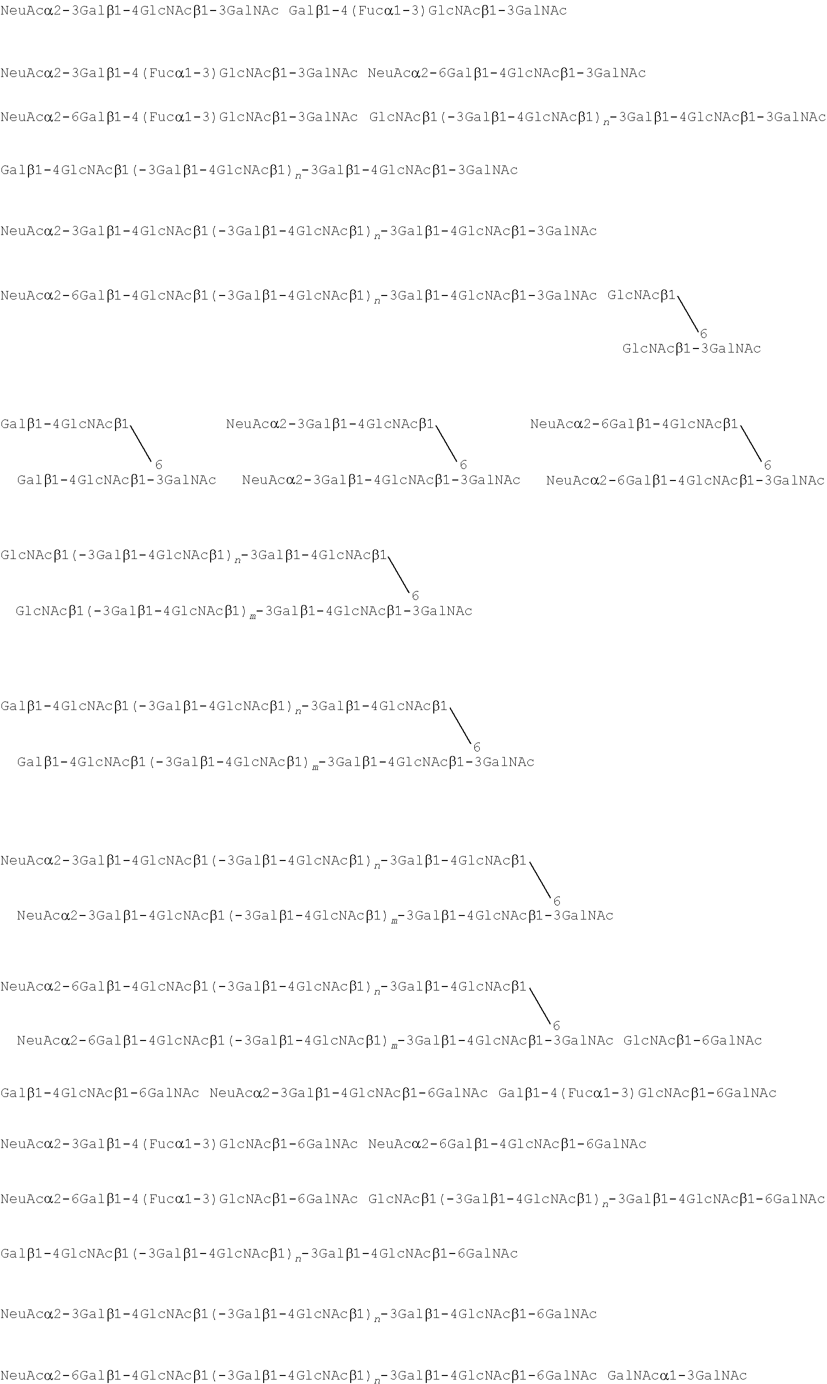

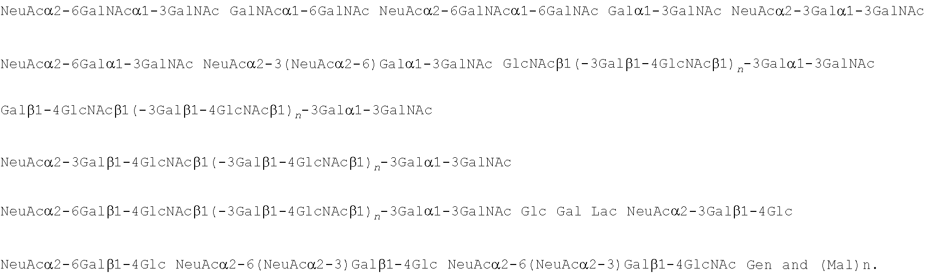

10. The glycosylated GLP-1 related peptide of claim 3, wherein the glycochain is selected from 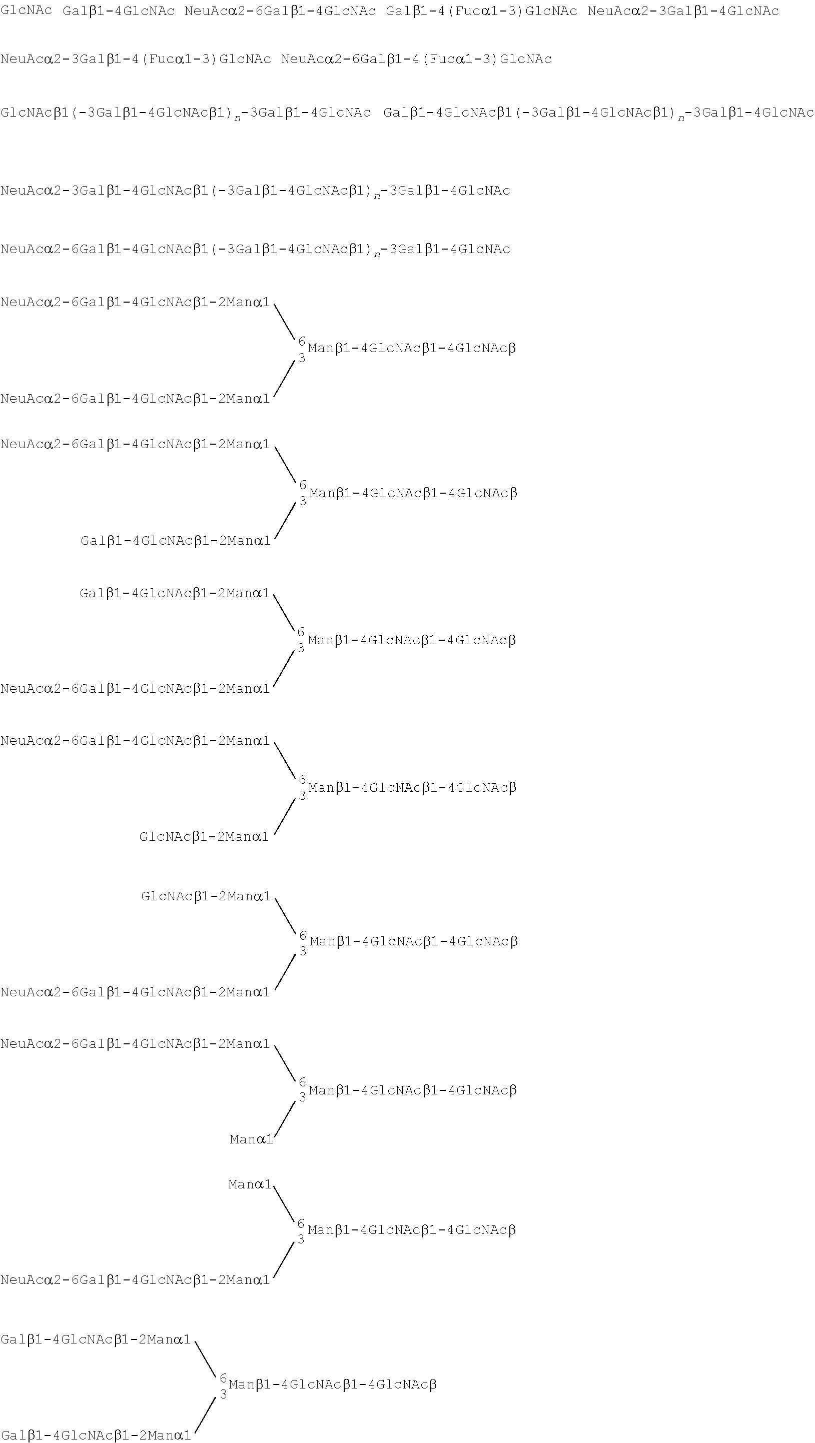 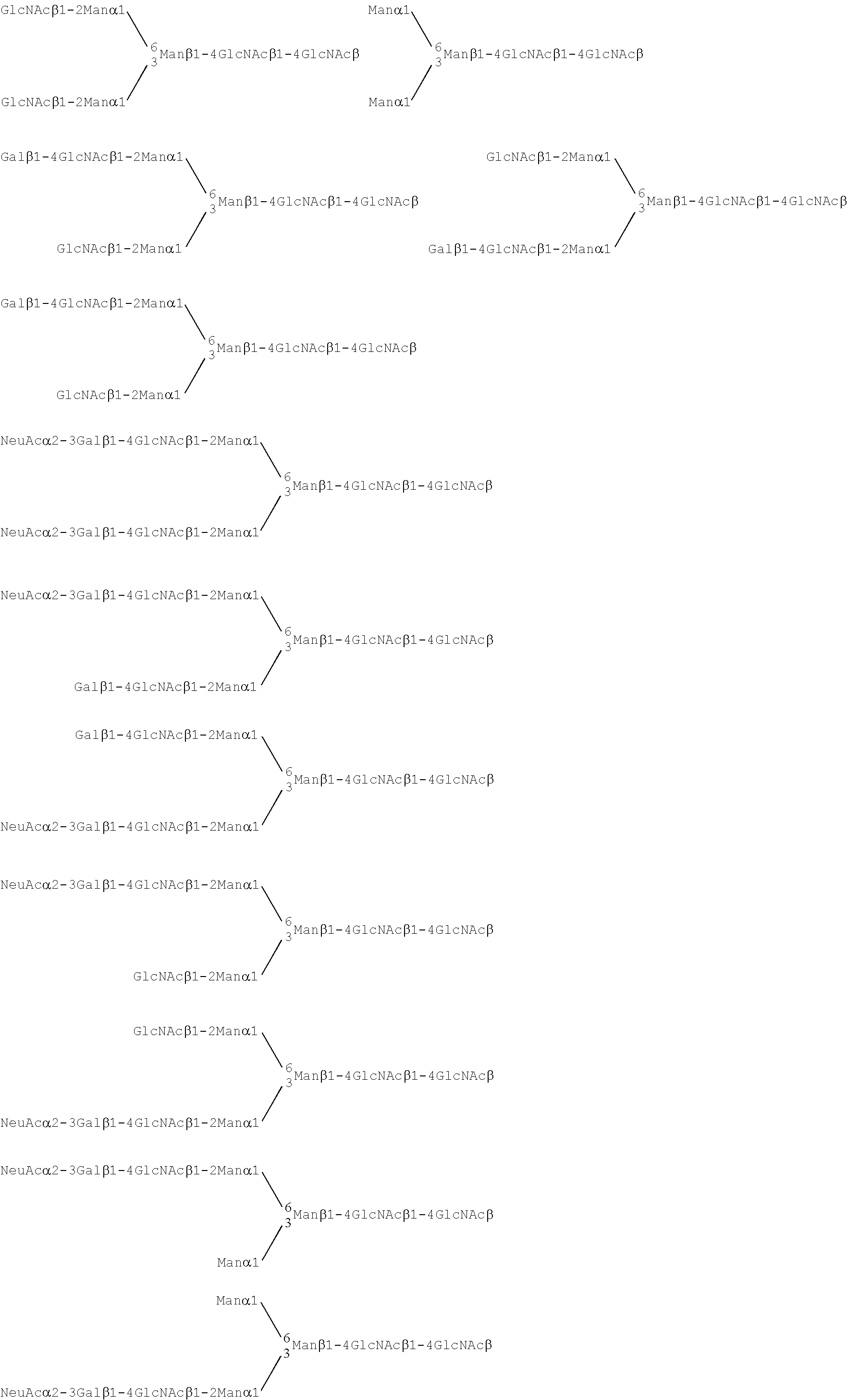
NeuAcα2-6(NeuAca2-3)Galβ1-4GlcNAc and Gen, and
the glycosylated amino acid residue is selected from the formula (c), (d), (g), (g′) and (j) below; 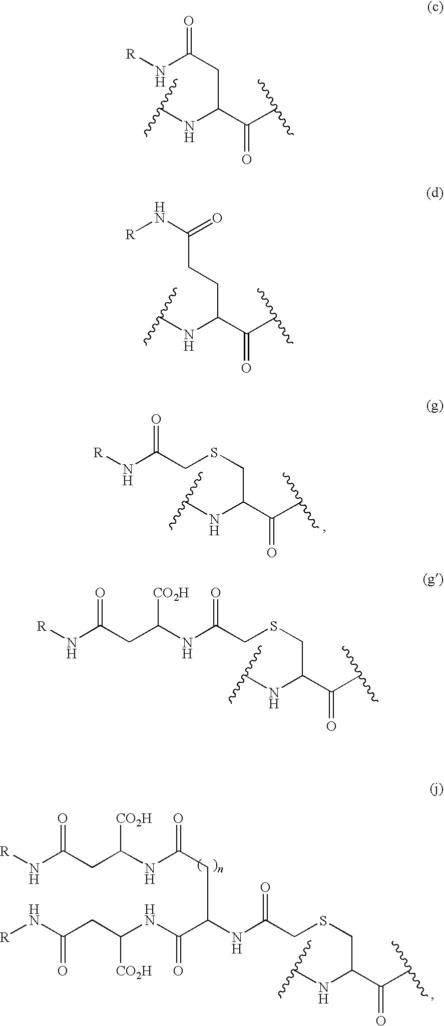
wherein n is an integer of 1 to 10.

11. A pharmaceutical composition comprising the glycosylated GLP-1 related peptide of claim 3 as an effective ingredient.

12. Medicine for treating or preventing diabetes comprising the glycosylated GLP-1 related peptide of claim 3 as an effective ingredient.


SEQUENCE LISTING (59 sequences)

SEQ ID NO: 1 - protein, 30 aa, Artificial Sequence
  Synthetic Construct
HAEGTFTSDVSSYLEGQAAKEFIAWLVKGR

SEQ ID NO: 2 - protein, 30 aa, Artificial Sequence
  Synthetic Construct
HNEGTFTSDVSSYLEGQAAKEFIAWLVKGR

SEQ ID NO: 3 - protein, 30 aa, Artificial Sequence
  Synthetic Construct
HAEGTFTSDVSSNLEGQAAKEFIAWLVKGR

SEQ ID NO: 4 - protein, 30 aa, Artificial Sequence
  Synthetic Construct
HAEGTFTSDVSSYLEGQAANEFIAWLVKGR

SEQ ID NO: 5 - protein, 30 aa, Artificial Sequence
  Synthetic Construct
HAEGTFTSDVSSYLEGQAAKEFIAWLVNGR

SEQ ID NO: 6 - protein, 31 aa, Artificial Sequence
  Synthetic Construct
HAEGTFTSDVSSYLEGQAAKEFIAWLVKGRN

SEQ ID NO: 7 - protein, 37 aa, Artificial Sequence
  Synthetic Construct
HAEGTFTSDVSSYLEGQAAKEFIAWLVKGRSGSGSGD

SEQ ID NO: 8 - protein, 30 aa, Artificial Sequence
  Synthetic Construct
HAEGTFTSDVSSYLEGQAANEFIAWLVNGR

SEQ ID NO: 9 - protein, 31 aa, Artificial Sequence
  Synthetic Construct
HAEGTFTSDVSSYLEGQAANEFIAWLVKGRN

SEQ ID NO: 10 - protein, 31 aa, Artificial Sequence
  Synthetic Construct
HAEGTFTSDVSSYLEGQAAKEFIAWLVNGRN

SEQ ID NO: 11 - protein, 31 aa, Artificial Sequence
  Synthetic Construct
HAEGTFTSDVSSYLEGQAAKEFIAWLVCGRC

SEQ ID NO: 12 - protein, 31 aa, Artificial Sequence
  Synthetic Construct
HAEGTFTSDVSSYLEGQAANEFIAWLVNGRN

SEQ ID NO: 13 - protein, 39 aa, Artificial Sequence
  Synthetic Construct
HGEGTFTSDLSKQMEEEAVRLFIEWLKNGGPSSGAPPPS

SEQ ID NO: 14 - protein, 41 aa, Artificial Sequence
  Synthetic Construct
NHGEGTFTSDLSKQMEEEAVRLFIEWLKNGGPSSSGAPPPS

SEQ ID NO: 15 - protein, 39 aa, Artificial Sequence
  Synthetic Construct
NGEGTFTSDLSKQMEEEAVRLFIEWLKNGGPSSGAPPPS

SEQ ID NO: 16 - protein, 39 aa, Artificial Sequence
  Synthetic Construct
HNEGTFTSDLSKQMEEEAVRLFIEWLKNGGPSSGAPPPS

SEQ ID NO: 17 - protein, 39 aa, Artificial Sequence
  Synthetic Construct
HGNGTFTSDLSKQMEEEAVRLFIEWLKNGGPSSGAPPPS

SEQ ID NO: 18 - protein, 39 aa, Artificial Sequence
  Synthetic Construct
HGENTFTSDLSKQMEEEAVRLFIEWLKNGGPSSGAPPPS

SEQ ID NO: 19 - protein, 39 aa, Artificial Sequence
  Synthetic Construct
HGEGNFTSDLSKQMEEEAVRLFIEWLKNGGPSSGAPPPS

SEQ ID NO: 20 - protein, 39 aa, Artificial Sequence
  Synthetic Construct
HGEGTNTSDLSKQMEEEAVRLFIEWLKNGGPSSGAPPPS

SEQ ID NO: 21 - protein, 39 aa, Artificial Sequence
  Synthetic Construct
HGEGTFNSDLSKQMEEEAVRLFIEWLKNGGPSSGAPPPS

SEQ ID NO: 22 - protein, 39 aa, Artificial Sequence
  Synthetic Construct
HGEGTFTNDLSKQMEEEAVRLFIEWLKNGGPSSGAPPPS

SEQ ID NO: 23 - protein, 39 aa, Artificial Sequence
  Synthetic Construct
HGEGTFTSNLSKQMEEEAVRLFIEWLKNGGPSSGAPPPS

SEQ ID NO: 24 - protein, 39 aa, Artificial Sequence
  Synthetic Construct
HGEGTFTSDNSKQMEEEAVRLFIEWLKNGGPSSGAPPPS

SEQ ID NO: 25 - protein, 39 aa, Artificial Sequence
  Synthetic Construct
HGEGTFTSDLNKQMEEEAVRLFIEWLKNGGPSSGAPPPS

SEQ ID NO: 26 - protein, 39 aa, Artificial Sequence
  Synthetic Construct
HGEGTFTSDLSNQMEEEAVRLFIEWLKNGGPSSGAPPPS

SEQ ID NO: 27 - protein, 39 aa, Artificial Sequence
  Synthetic Construct
HGEGTFTSDLSKNMEEEAVRLFIEWLKNGGPSSGAPPPS

SEQ ID NO: 28 - protein, 39 aa, Artificial Sequence
  Synthetic Construct
HGEGTFTSDLSKQNEEEAVRLFIEWLKNGGPSSGAPPPS

SEQ ID NO: 29 - protein, 39 aa, Artificial Sequence
  Synthetic Construct
HGEGTFTSDLSKQMNEEAVRLFIEWLKNGGPSSGAPPPS

SEQ ID NO: 30 - protein, 39 aa, Artificial Sequence
  Synthetic Construct
HGEGTFTSDLSKQMENEAVRLFIEWLKNGGPSSGAPPPS

SEQ ID NO: 31 - protein, 39 aa, Artificial Sequence
  Synthetic Construct
HGEGTFTSDLSKQMEENAVRLFIEWLKNGGPSSGAPPPS

SEQ ID NO: 32 - protein, 39 aa, Artificial Sequence
  Synthetic Construct
HGEGTFTSDLSKQMEEENVRLFIEWLKNGGPSSGAPPPS

SEQ ID NO: 33 - protein, 39 aa, Artificial Sequence
  Synthetic Construct
HGEGTFTSDLSKQMEEEANRLFIEWLKNGGPSSGAPPPS

SEQ ID NO: 34 - protein, 39 aa, Artificial Sequence
  Synthetic Construct
HGEGTFTSDLSKQMEEEAVNLFIEWLKNGGPSSGAPPPS

SEQ ID NO: 35 - protein, 39 aa, Artificial Sequence
  Synthetic Construct
HGEGTFTSDLSKQMEEEAVRNFIEWLKNGGPSSGAPPPS

SEQ ID NO: 36 - protein, 39 aa, Artificial Sequence
  Synthetic Construct
HGEGTFTSDLSKQMEEEAVRLNIEWLKNGGPSSGAPPPS

SEQ ID NO: 37 - protein, 39 aa, Artificial Sequence
  Synthetic Construct
HGEGTFTSDLSKQMEEEAVRLFNEWLKNGGPSSGAPPPS

SEQ ID NO: 38 - protein, 39 aa, Artificial Sequence
  Synthetic Construct
HGEGTFTSDLSKQMEEEAVRLFINWLKNGGPSSGAPPPS

SEQ ID NO: 39 - protein, 39 aa, Artificial Sequence
  Synthetic Construct
HGEGTFTSDLSKQMEEEAVRLFIENLKNGGPSSGAPPPS

SEQ ID NO: 40 - protein, 39 aa, Artificial Sequence
  Synthetic Construct
HGEGTFTSDLSKQMEEEAVRLFIEWNKNGGPSSGAPPPS

SEQ ID NO: 41 - protein, 39 aa, Artificial Sequence
  Synthetic Construct
HGEGTFTSDLSKQMEEEAVRLFIEWLNNGGPSSGAPPPS

SEQ ID NO: 42 - protein, 39 aa, Artificial Sequence
  Synthetic Construct
HGEGTFTSDLSKQMEEEAVRLFIEWLKNNGPSSGAPPPS

SEQ ID NO: 43 - protein, 39 aa, Artificial Sequence
  Synthetic Construct
HGEGTFTSDLSKQMEEEAVRLFIEWLKNGNPSSGAPPPS

SEQ ID NO: 44 - protein, 39 aa, Artificial Sequence
  Synthetic Construct
HGEGTFTSDLSKQMEEEAVRLFIEWLKNGGNSSGAPPPS

SEQ ID NO: 45 - protein, 39 aa, Artificial Sequence
  Synthetic Construct
HGEGTFTSDLSKQMEEEAVRLFIEWLKNGGPNSGAPPPS

SEQ ID NO: 46 - protein, 39 aa, Artificial Sequence
  Synthetic Construct
HGEGTFTSDLSKQMEEEAVRLFIEWLKNGGPSNGAPPPS

SEQ ID NO: 47 - protein, 39 aa, Artificial Sequence
  Synthetic Construct
HGEGTFTSDLSKQMEEEAVRLFIEWLKNGGPSSNAPPPS

SEQ ID NO: 48 - protein, 39 aa, Artificial Sequence
  Synthetic Construct
HGEGTFTSDLSKQMEEEAVRLFIEWLKNGGPSSGNPPPS

SEQ ID NO: 49 - protein, 39 aa, Artificial Sequence
  Synthetic Construct
HGEGTFTSDLSKQMEEEAVRLFIEWLKNGGPSSGANPPS

SEQ ID NO: 50 - protein, 39 aa, Artificial Sequence
  Synthetic Construct
HGEGTFTSDLSKQMEEEAVRLFIEWLKNGGPSSGAPNPS

SEQ ID NO: 51 - protein, 39 aa, Artificial Sequence
  Synthetic Construct
HGEGTFTSDLSKQMEEEAVRLFIEWLKNGGPSSGAPPDS

SEQ ID NO: 52 - protein, 39 aa, Artificial Sequence
  Synthetic Construct
HGEGTFTSDLSKQMEEEAVRLFIEWLKNGGPSSGAPPPD

SEQ ID NO: 53 - protein, 40 aa, Artificial Sequence
  Synthetic Construct
HGEGTFTSDLSKQMEEEAVRLFIEWLKNGGPSSGAPPPSN

SEQ ID NO: 54 - protein, 40 aa, Artificial Sequence; identical to SEQ ID NO: 53
  Synthetic Construct

SEQ ID NO: 55 - protein, 40 aa, Artificial Sequence
  Synthetic Construct
HGEGTFTSDLSKQMEENAVRNFIEWLKNGGPSSGAPPPSN

SEQ ID NO: 56 - protein, 28 aa, Artificial Sequence
  Synthetic Construct
HGEGTFTSDLSKQMEENAVRNFIEWLKN

SEQ ID NO: 57 - protein, 39 aa, Artificial Sequence
  Synthetic Construct
HGEGTFTSDLSKQMEENAVRNFIEWLKCGGPSSGAPPPS

SEQ ID NO: 58 - protein, 40 aa, Artificial Sequence
  Synthetic Construct
HGEGTFTSDLSKQMEENAVRNFIEWLKCGGPSSGAPPPSC

SEQ ID NO: 59 - protein, 40 aa, Artificial Sequence
  Synthetic Construct
HGEGTFTSDLSKQMEENAVRCFIEWLKCGGPSSGCPPPSC